(19) United States
(12) Patent Application Publication
(10) Pub. No.: US 2024/0058352 A1
(43) Pub. Date: Feb. 22, 2024

(54) COMBINATION THERAPIES FOR THE TREATMENT OF CANCER

(71) Applicant: Erasca, Inc., San Diego, CA (US)

(72) Inventors: Leenus MARTIN, San Diego, CA (US); Leslie Harris BRAIL, Newton, MA (US); Robert Field SHOEMAKER, San Diego, CA (US)

(21) Appl. No.: 18/256,090

(22) PCT Filed: Dec. 10, 2021
(86) PCT No.: PCT/US2021/062921

§ 371 Date: Jun. 6, 2023

Related U.S. Application Data
(60) Provisional application No. 63/124,674, filed on Dec. 11, 2020.
(60) Provisional application No. 63/124,671, filed on Dec. 11, 2020.
(60) Provisional application No. 63/124,667, filed on Dec. 11, 2020.
(60) Provisional application No. 63/124,663, filed on Dec. 11, 2020.

(51) Int. Cl. A61K 31/5383; A61K 31/498; A61P 35/00; A61K 31/4545; A61K 31/519; A61K 31/4184; A61K 31/506

(52) U.S. Cl. CPC A61K 31/5383; A61K 31/498; A61P 35/00; A61K 31/4545; A61K 31/519; A61K 31/4184; A61K 31/506

(57) ABSTRACT

The present disclosure provides methods of treating cancer with a combination therapies of a SHP2 inhibitor, such as the compound of Formula I, and an FGFR inhibitor, a B-Raf inhibitor, a MEK inhibitor, or a MET inhibitor

### CROSS-REFERENCE

This application claims the benefit of U.S. Provisional Patent Application No. 63/124,663 filed Dec. 11, 2020; U.S. Provisional Patent Application No. 63/124,667 filed Dec. 11, 2020; U.S. Provisional Patent Application No. 63/124,671 filed Dec. 11, 2020; and U.S. Provisional Patent Application No. 63/124,674 filed Dec. 11, 2020; each of which is incorporated herein by reference in its entirety.

### BACKGROUND OF THE INVENTION

Src Homology-2 phosphatase (SHP2) is a non-receptor protein phosphatase ubiquitously expressed in various tissues and cell types (see reviews: Tajan M et al., Eur J Med Genet 2016 58(10):509-25; Grossmann K S et al., Adv Cancer Res 2010 106:53-89). SHP2 is composed of two Src homology 2 (N-SH2 and C-SH2) domains in its NH2-terminus, a catalytic PTP (protein-tyrosine phosphatase) domain, and a C-terminal tail with regulatory properties. At the basal state, the intermolecular interactions between the SH2 domains and the PTP domain prevent the access of substrates to the catalytic pocket, keeping SHP2 into a closed, auto-inhibited conformation. In response to stimulation, SHP2 activating proteins bearing phosphor-tyrosine motifs bind to the SH2 domains, leading to exposure of active site and enzymatic activation of SHP2.

### SUMMARY OF THE INVENTION

The present embodiments disclosed herein generally relate to compositions and methods related to combination therapies to treat cancer utilizing a SHP2 inhibitor in conjunction with an FGFR inhibitor, a B-Raf inhibitor, a MEK inhibitor or a MET inhibitor, including while providing an unexpected degree synergy.

SHP2 plays important roles in fundamental cellular functions including proliferation, differentiation, cell cycle maintenance and motility. By dephosphorylating its associated signaling molecules, SHP2 regulates multiple intracellular signaling pathways in response to a wide range of growth factors, cytokines, and hormones. Cell signaling processes in which SHP2 participates include the RAS-MAPK (mitogen-activated protein kinase), the PI3K (phosphoinositol 3-kinase)-AKT, and the JAK-STAT pathways.

SHP2 also plays a signal-enhancing role on this pathway, acting downstream of RTKs and upstream of RAS. One common mechanism of resistance involves activation of RTKs that fuel reactivation of the MAPK signaling. RTK activation recruits SHP2 via direct binding and through adaptor proteins. Those interactions result in the conversion of SHP2 from the closed (inactive) conformation to open (active) conformation. SHP2 is an important facilitator of RAS signaling reactivation that bypasses pharmacological inhibition in both primary and secondary resistance. Inhibition of SHP2 achieves the effect of globally attenuating upstream RTK signaling that often drives oncogenic signaling and adaptive tumor escape (see Prahallad, A. et al. Cell Reports 12, 1978-1985 (2015); Chen Y N, Nature 535, 148-152(2016)), which is incorporated herein by reference in its entirety for all of its teachings, including without limitation all methods, compounds, compositions, data and the like, for use with any of the embodiments and disclosure herein.

Fibroblast growth factor receptors (FGFR) bind to members of the fibroblast growth factor family of proteins, also impact the RAS-MAPK signal transduction pathway upstream of RAS. The opportunity to target signal transduction pathways from multiple angles and potentially ameliorate feedback loops upstream of Ras via SHP2 provides opportunities for developing methods that employ combination therapies. The present disclosure provides such methods while providing an unexpected degree synergy.

The RAS-MAPK signal transduction pathway includes the Raf family of proteins. The family includes composed of three related kinases (A-, B- and C-Raf) that act as downstream effectors of Ras. B-Raf, in particular is a serine/threonine protein kinase that activates the MAP kinase/ERK-signaling pathway. Constitutively active B-Raf mutants are commonly known to cause cancer by excessively signaling cells to grow. For example, activating B-Raf V600E kinase mutations occur in about 7% of human malignancies and about 50-60% of melanomas.

The RAS-MAPK signal transduction pathway also includes MEK1 and MEK2. MEK1 and MEK2 are dual function serine/threonine and tyrosine protein kinases, also known as MAP kinase kinases. MEK plays a pivotal role in the RAS-regulated RAF-MEK-ERK signaling pathway, a pathway which transmits signals from growth factor receptors to the nucleus to regulate, inter alia, cell proliferation, differentiation, survival and invasion.

Lastly, extracellular MET (or c-MET), which is a pivotal protein tyrosine kinase, operates upstream of the RAS-MAPK signal transduction pathway. The opportunity to target signal transduction pathways from multiple angles and potentially ameliorate feedback loops upstream of Ras via SHP2 provides opportunities for developing methods that employ combination therapies. The present embodiments disclosed herein provide such methods while providing an unexpected degree synergy.

In a first aspect, the present disclosure provides a method of treating a subject having cancer comprising administering to the subject a therapeutically effective amount of a compound of Formula I or its pharmaceutically acceptable salt:

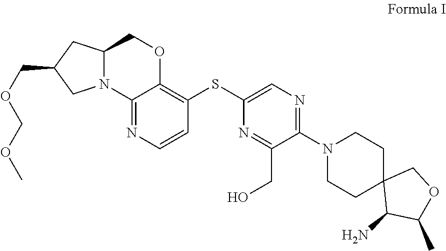 [SMILES: COCOC[C@H]1C[C@H]2COc3c(Sc4cnc(N5CCC6(CC5)CO[C@@H](C)[C@H]6N)c(CO)n4)ccnc3N2C1]

in combination with an FGFR inhibitor. In some embodiments, the FGFR in the subject is constitutively active. In some embodiments, the cancer lung cancer. In some embodiments, the cancer is hepatocellular carcinoma. In some embodiments, the cancer is cholangiocarcinoma. In some embodiments, the cancer is pancreatic ductal adenocarcinoma (PDAC). In some embodiments, the inhibitor is selected from the group consisting of erdafitinib, AZD4547, Ly2874455, CH5183284, NVP-BGJ398, INCB054828, rogaratinib, PRN1371, TAS-120, BLU-554, H3B-6527, andFGF401. In some embodiments, the FGFR inhibitor is erdafitinib. In some embodiments, the FGFR inhibitor is pemigatinib, infigratinib, dovitinib, ponatinib, nintedanib, and fisogatinib. In some embodiments, the method comprises administering a third MAPK pathway inhibitor. In some embodiments, the administration is oral. In some embodiments, the dosing of the compound of Formula I is in a range from 20 mg to 400 mg daily. In some embodiments, the dosing of the FGFR inhibitor is in a range from 1 mg to 500 mg daily.

In a second aspect, the present disclosure provides a method of treating liver cancer in a subject comprising orally administering to the subject a therapeutically effective amount of a compound of Formula I or its pharmaceutically acceptable salt:

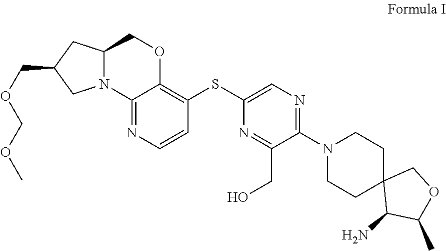 [SMILES: COCOC[C@H]1C[C@H]2COc3c(Sc4cnc(N5CCC6(CC5)CO[C@@H](C)[C@H]6N)c(CO)n4)ccnc3N2C1]

in combination with erdafitinib. In some embodiments, the compound of Formula I is administered once or twice daily. In some embodiments, erdafitinib is administered once or twice daily. In some embodiments, the subject is a human.

In a third aspect, the present disclosure provides a kit comprising a compound of Formula I or a pharmaceutically acceptable salt thereof and an FGFR inhibitor. In some embodiments, the compound of Formula I and the FGFR inhibitor are in separate packages. In some embodiments, the kit further comprises instructions to administer the contents of the kit to a subject for the treatment of cancer. In some embodiments, the FGFR inhibitor is one or more of erdafitinib, AZD4547, Ly2874455, CH5 183284, NVP-BGJ398, INCB054828, rogaratinib, PRN1371, TAS-120, BLU-554, H3B-6527, FGF401, pemigatinib, infigratinib, dovitinib, ponatinib, nintedanib, and fisogatinib.

In a fourth aspect, the present disclosure provides a method of treating a subject having cancer comprising administering to the subject a therapeutically effective amount of a compound of Formula I or its pharmaceutically acceptable salt:

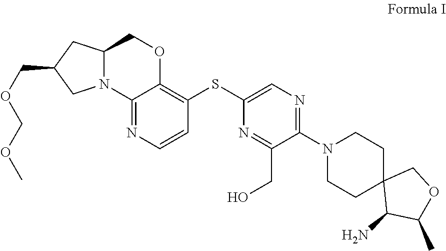 [SMILES: COCOC[C@H]1C[C@H]2COc3c(Sc4cnc(N5CCC6(CC5)CO[C@@H](C)[C@H]6N)c(CO)n4)ccnc3N2C1]

in combination with an inhibitor of a B-Raf protein having a class 1 mutation. In some embodiments, the class 1 mutation is V600E. In some embodiments, the cancer is colorectal cancer. In some embodiments, the cancer is melanoma. In some embodiments, the cancer is thyroid cancer. In some embodiments, the cancer is pancreatic ductal adenocarcinoma (PDAC). In some embodiments, the inhibitor is selected from the group consisting of encorafenib, vemurafenib, dabrafenib, sorafenib, and regorafenib. In some embodiments, the inhibitor is encorafenib. In some embodiments, the inhibitor is vemurafenib. In some embodiments, the inhibitor is dabrafenib. In some embodiments, the inhibitor is sorafenib. In some embodiments, the inhibitor is regorafenib. In some embodiments, the method comprises administering a third MAPK pathway inhibitor. In some embodiments, the administration is oral. In some embodiments, the dosing of the compound of Formula I is in a range from 20 mg to 400 mg daily. In some embodiments, the dosing of the B-Raf inhibitor is in a range from 1 mg to 500 mg.

In another aspect, the present disclosure provides a method of treating colorectal cancer in a subject comprising orally administering to the subject a therapeutically effective amount of a compound of Formula I or its pharmaceutically acceptable salt:

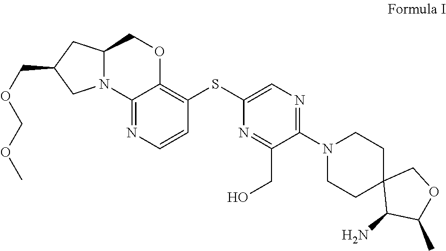 [SMILES: COCOC[C@H]1C[C@H]2COc3c(Sc4cnc(N5CCC6(CC5)CO[C@@H](C)[C@H]6N)c(CO)n4)ccnc3N2C1]

in combination with B-Raf inhibitor encorafenib. In some embodiments, the compound of Formula I is administered once or twice daily. In some embodiments, encorafenib is administered once or twice daily. In some embodiments, the subject is human.

In another aspect, the present disclosure provides a method of treating a subject having cancer comprising administering to the subject a therapeutically effective amount of a compound of Formula I or its pharmaceutically acceptable salt:

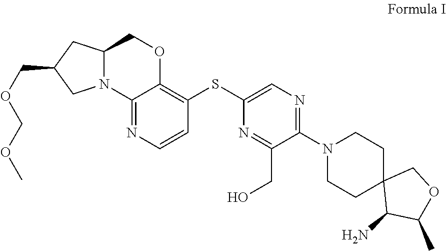 [SMILES: COCOC[C@H]1C[C@H]2COc3c(Sc4cnc(N5CCC6(CC5)CO[C@@H](C)[C@H]6N)c(CO)n4)ccnc3N2C1]

in combination with encorafenib.

In another aspect, the present disclosure provides a method of treating a subject having cancer comprising administering to the subject a therapeutically effective amount of a compound of Formula I or its pharmaceutically acceptable salt:

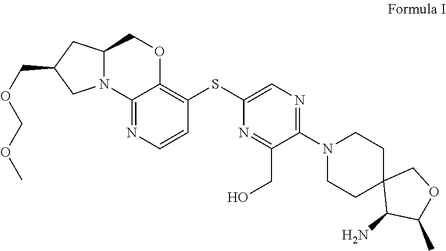 [SMILES: COCOC[C@H]1C[C@H]2COc3c(Sc4cnc(N5CCC6(CC5)CO[C@@H](C)[C@H]6N)c(CO)n4)ccnc3N2C1]

in combination with vemurafenib

In another aspect, the present disclosure provides a method of treating a subject having cancer comprising administering to the subject a therapeutically effective amount of a compound of Formula I or its pharmaceutically acceptable salt:

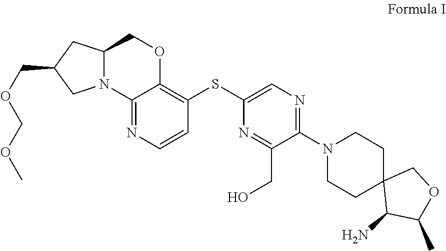 [SMILES: COCOC[C@H]1C[C@H]2COc3c(Sc4cnc(N5CCC6(CC5)CO[C@@H](C)[C@H]6N)c(CO)n4)ccnc3N2C1]

in combination with dabrafenib

In another aspect, the present disclosure provides a method of treating a subject having cancer comprising administering to the subject a therapeutically effective amount of a compound of Formula I or its pharmaceutically acceptable salt:

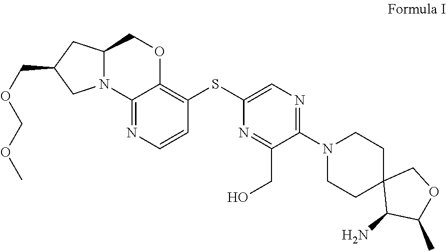 [SMILES: COCOC[C@H]1C[C@H]2COc3c(Sc4cnc(N5CCC6(CC5)CO[C@@H](C)[C@H]6N)c(CO)n4)ccnc3N2C1]

in combination with sorafenib.

In another aspect, the present disclosure provides a method of treating a subject having cancer comprising administering to the subject a therapeutically effective amount of a compound of Formula I or its pharmaceutically acceptable salt:

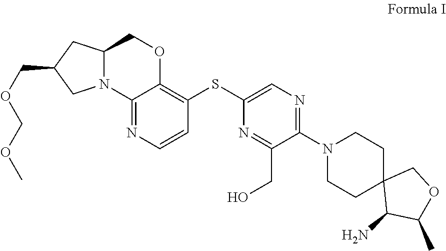 [SMILES: COCOC[C@H]1C[C@H]2COc3c(Sc4cnc(N5CCC6(CC5)CO[C@@H](C)[C@H]6N)c(CO)n4)ccnc3N2C1]

in combination with regorafenib.

In various embodiments of the methods described herein, the cancer is colorectal cancer. In some embodiments, the cancer is thyroid cancer. In some embodiments, the cancer is melanoma. In some embodiments, the cancer is pancreatic ductal adenocarcinoma (PDAC) In some embodiments, a dosing of the B-Raf inhibitor is less than a dosing required for a monotherapy with the B-Raf inhibitor. In some embodiments, a dosing of the compound of Formula I is less than a dosing required for a monotherapy with the compound of Formula I.

In another aspect, the present disclosure provides a method of inhibiting ERK1/2 phosphorylation in a cell population comprising contacting a cell population with the compound of Formula I or its pharmaceutically acceptable salt:

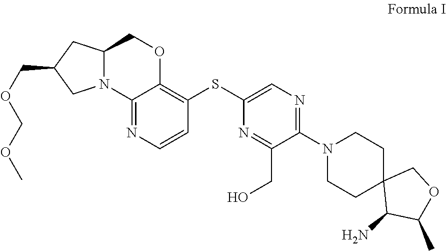 [SMILES: COCOC[C@H]1C[C@H]2COc3c(Sc4cnc(N5CCC6(CC5)CO[C@@H](C)[C@H]6N)c(CO)n4)ccnc3N2C1]

in combination with regorafenib. In some embodiments, a concentration of the compound of Formula I is a range from 1 nM to 500 nM. In some embodiments, a concentration of encorafenib is in a range from 10 nM to 20 nM.

In another aspect, the present disclosure provides a kit comprising a compound of Formula I or a pharmaceutically acceptable salt thereof and a B-Raf inhibitor. In some embodiments, the compound of Formula I and the B-Raf inhibitors are in separate packages. In some embodiments, the kit further comprises instructions to administer the contents of the kit to a subject for the treatment of cancer. In some embodiments, the B-Raf inhibitor is one or more of encorafenib, vemurafenib, dabrafenib, sorafenib, and regorafenib.

In another aspect, the present disclosure provides a method of treating a subject having cancer comprising administering to the subject a therapeutically effective amount of a compound of Formula I or its pharmaceutically acceptable salt:

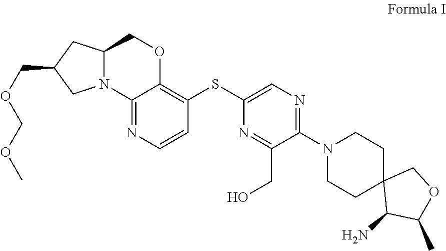 [SMILES: COCOC[C@H]1C[C@H]2COc3c(Sc4cnc(N5CCC6(CC5)CO[C@@H](C)[C@H]6N)c(CO)n4)ccnc3N2C1]

in combination with a MEK inhibitor. In some embodiments, the MEK inhibitor inhibits MEK1 selectively or MEK2 selectively or both MEK1 and MEK2 selectively. In some embodiments, the cancer is metastatic. In some embodiments, the cancer colorectal cancer. In some embodiments, the cancer is melanoma. In some embodiments, the cancer is lung cancer. In some embodiments, the cancer is pancreatic cancer. In some embodiments, the cancer is breast cancer. In some embodiments, the cancer is pancreatic ductal adenocarcinoma (PDAC). In some embodiments, the MEK inhibitor is selected from the group consisting of trametinib, cobimetinib, binimetinib, PD-0325901, selumetinib and CI-1040. In some embodiments, the MEK inhibitor is trametinib. In some embodiments, the MEK inhibitor is cobimetinib. In some embodiments, the MEK inhibitor is binimetinib. In some embodiments, the MEK inhibitor is PD-325901. In some embodiments, the MEK inhibitor is CI-1040. In some embodiments, the method comprises administering a further MAPK pathway inhibitor. In some embodiments, the administration is oral. In some embodiments, the dosing of the compound of Formula I is in a range from 20 mg to 400 mg daily. In some embodiments, the dosing of the MEK inhibitor is in a range from 1 mg to 500 mg daily.

In another aspect, the present disclosure provides a method of treating cancer in a subject comprising orally administering to the subject a therapeutically effective amount of a compound of Formula I or its pharmaceutically acceptable salt:

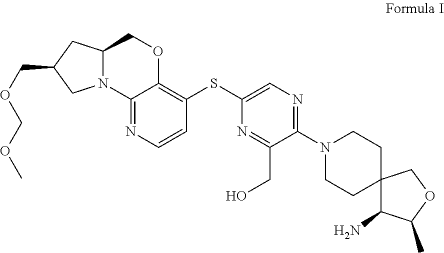 [SMILES: COCOC[C@H]1C[C@H]2COc3c(Sc4cnc(N5CCC6(CC5)CO[C@@H](C)[C@H]6N)c(CO)n4)ccnc3N2C1]

in combination with MEK inhibitor binimetinib or trametinib. In some embodiments, the compound of Formula I is administered once or twice daily. In some embodiments, binimetinib or trametinib is administered once or twice daily. In some embodiments, the subject is a human.

In another aspect, the present disclosure provides a method of treating a subject having cancer comprising administering to the subject a therapeutically effective amount of a compound of Formula I or its pharmaceutically acceptable salt:

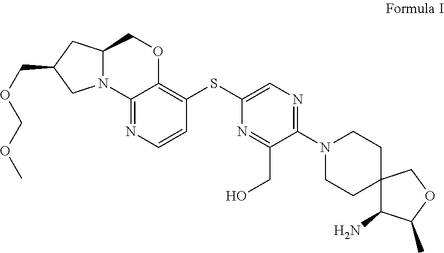 [SMILES: COCOC[C@H]1C[C@H]2COc3c(Sc4cnc(N5CCC6(CC5)CO[C@@H](C)[C@H]6N)c(CO)n4)ccnc3N2C1]

in combination with binimetinib.

In another aspect, the present disclosure provides a method of treating a subject having cancer comprising administering to the subject a therapeutically effective amount of a compound of Formula I or its pharmaceutically acceptable salt:

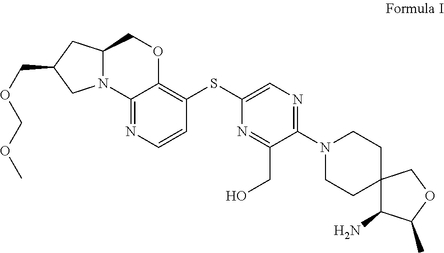 [SMILES: COCOC[C@H]1C[C@H]2COc3c(Sc4cnc(N5CCC6(CC5)CO[C@@H](C)[C@H]6N)c(CO)n4)ccnc3N2C1]

in combination with trametinib.

In various embodiments of the methods described herein, the cancer is colorectal cancer. In some embodiments, the cancer is lung cancer. In some embodiments, the cancer is melanoma. In some embodiments, a dosing of the MEK inhibitor is less than a dosing required for a monotherapy with the MEK inhibitor. In some embodiments, a dosing of the compound of Formula I is less than a dosing required for a monotherapy with the compound of Formula I.

In another aspect, the present disclosure provides a method of inhibiting ERK1/2 phosphorylation comprising contacting a cell population with Formula I or its pharmaceutically acceptable salt:

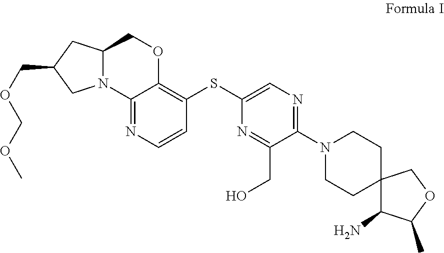 [SMILES: COCOC[C@H]1C[C@H]2COc3c(Sc4cnc(N5CCC6(CC5)CO[C@@H](C)[C@H]6N)c(CO)n4)ccnc3N2C1]

in combination with binimetinib or trametinib. In some embodiments, a concentration of the compound of Formula I is in a range from 1 nM to 1,000 nM. In some embodiments, a concentration of MEK inhibitors is in a range from 10 nM to 500 nM.

In another aspect, the present disclosure provides a kit comprising a compound of Formula I or a pharmaceutically acceptable salt thereof and an MEK inhibitor. In some embodiments, the compound of Formula I and the MEK inhibitor are in separate packages. In some embodiments, the kit further comprises instructions to administer the contents of the kit to a subject for the treatment of cancer. In some embodiments, the MEK inhibitor is one or more of trametinib or binimetinib.

In another aspect, the present disclosure provides a method of treating a subject having cancer comprising administering to the subject a therapeutically effective amount of a compound of Formula I or its pharmaceutically acceptable salt:

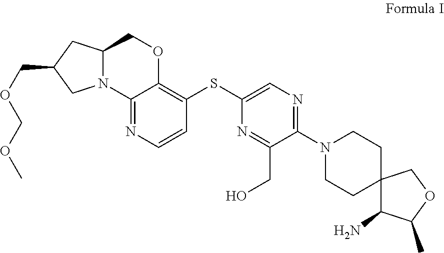 [SMILES: COCOC[C@H]1C[C@H]2COc3c(Sc4cnc(N5CCC6(CC5)CO[C@@H](C)[C@H]6N)c(CO)n4)ccnc3N2C1]

in combination with a MET inhibitor. In some embodiments, the MET inhibitor is also an ALK inhibitor, a ROS1 inhibitor, or both. In some embodiments, the cancer is non-small lung cancer. In some embodiments, the cancer is stomach cancer. In some embodiments, the cancer is gastric adenocarcinoma. In some embodiments, the cancer is pancreatic ductal adenocarcinoma (PDAC). In some embodiments, the MET inhibitor is selected from the group consisting of crizotinib, tepotinib, savolitinib, cabozantinib, and tivantinib. In some embodiments, the MET inhibitor is crizotinib. In some embodiments, the MET inhibitor is tepotinib. In some embodiments, the inhibitor is savolitinib. In some embodiments, the inhibitor is cabozantinib. In some embodiments, the inhibitor is tivantinib. In some embodiments, the method comprises administering a third MAPK pathway inhibitor. In some embodiments, the administration is oral. In some embodiments, the dosing of the compound of Formula I is in a range from 10 mg to 500 mg daily. In some embodiments, the dosing of the inhibitor is in a range from 20 mg to 400 mg daily.

In another aspect, the present disclosure provides a method of treating stomach cancer in a subject comprising orally administering to the subject a therapeutically effective amount of a compound of Formula I or its pharmaceutically acceptable salt:

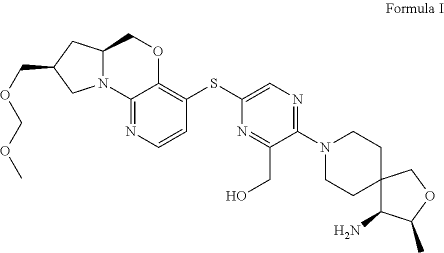 [SMILES: COCOC[C@H]1C[C@H]2COc3c(Sc4cnc(N5CCC6(CC5)CO[C@@H](C)[C@H]6N)c(CO)n4)ccnc3N2C1]

in combination with crizotinib. In some embodiments, the compound of Formula I is administered once or twice daily. In some embodiments, crizotinib is administered once or twice daily. In some embodiments, the subject is a human.

In a final aspect, the present disclosure provides a kit comprising a compound of Formula I or a pharmaceutically acceptable salt thereof and a MET inhibitor. In some embodiments, the compound of Formula I and the MET inhibitor are in separate packages. In some embodiments, the kit further comprises instructions to administer the contents of the kit to a subject for the treatment of cancer. In some embodiments, the MET inhibitor is one or more of crizotinib, tepotinib, savolitinib, cabozantinib, and tivantinib.

In some embodiments, the compound of Formula I, or a pharmaceutically acceptable salt thereof, is formulated as a pharmaceutical composition.

In some embodiments, the compound of Formula I, or a pharmaceutically acceptable salt thereof, is formulated as an oral composition.

In some embodiments, the compound of Formula I, or a pharmaceutically acceptable salt thereof, is administered once or twice a day.

In some embodiments, the compound of Formula I, or a pharmaceutically acceptable salt thereof, is administered over a continuous 28-day cycle.

In some embodiments, the compound of Formula I, or a pharmaceutically acceptable salt thereof, is administered once a day in the amount of about 10 mg to about 140 mg.

In some embodiments, the compound, or a pharmaceutically acceptable salt thereof, is administered once a day for a 3-week cycle, comprising 2 weeks of administration of the compound followed by 1 week of no administration of the compound.

In some embodiments, the compound of Formula I, or a pharmaceutically acceptable salt thereof, is administered once a day for a 4-week cycle, comprising 3 weeks of administration of the compound followed by 1 week of no administration of the compound.

In some embodiments, the compound of Formula I, or a pharmaceutically acceptable salt thereof, is administered over a period of 6 weeks.

In some embodiments, the compound of Formula I, or a pharmaceutically acceptable salt thereof, is administered over a period of 8 weeks.

In some embodiments, the compound of Formula I, or a pharmaceutically acceptable salt thereof, is administered 3 times a week.

In some embodiments, the compound of Formula I, or a pharmaceutically acceptable salt thereof, is administered on day 1, day 3, and day 5 of the week.

In some embodiments, the compound of Formula I, or a pharmaceutically acceptable salt thereof, is administered 4 times a week.

In some embodiments, the compound of Formula I, or a pharmaceutically acceptable salt thereof, is administered for a 3-week cycle, comprising 2 weeks of administration of the compound followed by 1 week of no administration of the compound.

In some embodiments, the compound of Formula I, or a pharmaceutically acceptable salt thereof, is administered for a 4-week cycle, comprising 3 weeks of administration of the compound followed by 1 week of no administration of the compound.

In some embodiments, the compound of Formula I, or a pharmaceutically acceptable salt thereof, is administered twice a day, two days per week.

In some embodiments, the compound of Formula I, or a pharmaceutically acceptable salt thereof, is administered over a period of 8 weeks.

In some embodiments, the compound of Formula I, or a pharmaceutically acceptable salt thereof, is administered on day 1 and day 2 of each week.

In some embodiments, the cancer is selected from lung cancer, stomach cancer, liver cancer, colon cancer, kidney cancer, breast cancer, pancreatic cancer, pancreatic ductal adenocarcinoma (PDAC), juvenile myelomonocytic leukemia, neurolastoma, melanoma, and acute myeloid leukemia.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1A shows data indicating that the combinations of the compound of Formula I and FGFR inhibitor erdafitinib exhibit synergy in vitro. This data indicates that there is a significant degree of synergy in the combination of the compound of Formula I and erdafitinib.

FIG. 1B shows a synergy data in Hep3B cancer cell line using the combination of the compound of Formula I and erdafitinib. This data indicates that there is a significant degree of synergy in the combination of the compound of Formula I and erdafitinib.

FIG. 2 shows a graph of tumor volume over a period of treatment time with the compound of Formula I alone, erdafitinib alone, and the combination of the compound of Formula I and erdafitinib in hepatoma carcinoma CDX model KATO III.

FIG. 3 shows a graph of tumor volume over a period of treatment time with the compound of Formula I alone, erdafitinib alone, and the combination of the compound of Formula I and erdafitinib in FGFR2 amplified gastric cancer CDX SNU-16.

FIG. 4 shows a graph of tumor volume over a period of treatment time with the compound of Formula I alone, erdafitinib alone, and the combination of the compound of Formula I and erdafitinib in FGF19-FGFR4 dependent liver cancer CDX model Huh-7.

FIG. 5 shows data indicating that the combination of the compound of Formula I and encorafenib exhibits synergy across multiple BRAF V600E mutated cells.

FIG. 6 shows a synergy data in RKO BRAF^{V600E} CRC cell line using the combination of the compound of Formula I and BRAF inhibitor encorafenib. This data indicates that there is a significant degree of synergy in the combination of the compound of Formula I and encorafenib.

FIG. 7 shows a synergy data in WiDr BRAF^{V600E} CRC cell line using the combination of the compound of Formula I and BRAF inhibitor encorafenib. This data indicates that there is a significant degree of synergy in the combination of the compound of Formula I and encorafenib.

FIG. 8 shows a synergy data in HT29 BRAF^{V600E} CRC cell line using the combination of the compound of Formula I and BRAF inhibitor encorafenib. This data indicates that there is a significant degree of synergy in the combination of the compound of Formula I and encorafenib.

FIG. 9A shows a gel indicating the synergistic inhibition of ERK1/2 phosphorylation in the RKO colorectal cancer cell line. FIG. 9A indicates a robust reduction of pERK1/2 using the combination of the compound of Formula I and encorafenib.

FIG. 9B shows a gel indicating the robust inhibition of ERK1/2 phosphorylation in the WiDr colorectal cancer cell line. FIG. 9B indicates a robust reduction of pERK1/2 using the combination of the compound of Formula I and encorafenib.

FIG. 9C shows a plot of the antiproliferation effect of the compound of Formula I alone or the compound of Formula I combined with encorafenib in the RKO colorectal cancer cell line. FIG. 9C suggests combination of the compound of Formula I and encorafenib increased inhibitory activity of the compound of Formula I.

FIG. 9D shows a plot of antiproliferation effect of the compound of Formula I or the compound of Formula I combined with encorafenib in the WiDr colorectal cancer cell line. FIG. 9D suggests combination of the compound of Formula I and encorafenib increased inhibitory activity of the compound of Formula I.

FIG. 10A shows a gel comparing the synergistic inhibition of ERK1/2 phosphorylation in the RKO colorectal cancer cell line with combinations: the compound of Formula I+encorafenib; TNO155+encorafenib; and RMC-4550+encorafenib, indicating that inhibition of ERK1/2 phosphorylation is most effective with the combination of SHP2 inhibitor compound of Formula I and encorafenib.

FIG. 10B shows a bar graph of pERK as a percentage of control for 1. Control; 2. (the compound of Formula I); 3. encorafenib; and 4. (the compound of Formula I)+encorafenib, indicating that inhibition of ERK1/2 phosphorylation is most effective with the combination of SHP2 inhibitor compound of Formula I and encorafenib.

FIG. 10C shows a bar graph of pERK as a percentage of control for 1. Control; 2. TNO155; 3. encorafenib; and 4. TNO155+encorafenib, indicating that inhibition of ERK1/2 phosphorylation is most effective with the combination of SHP2 inhibitor compound of Formula I and encorafenib.

FIG. 10D shows a bar graph of pERK as a percentage of control for 1. Control; 2. RMC-4550; 3. encorafenib; and 4. RMC-4550+encorafenib, indicating that inhibition of ERK1/2 phosphorylation is most effective with the combination of SHP2 inhibitor compound of Formula I and encorafenib.

FIG. 11 shows a graph of tumor volume over a period of treatment time with the compound of Formula I alone, encorafenib alone, and the combination of the compound of Formula I and encorafenib in BRAF^{V600E} mutant CRC PDX model CR0029.

FIG. 12 shows a graph of tumor volume over a period of treatment time with the compound of Formula I alone, encorafenib alone, and the combination of the compound of Formula I and encorafenib in BRAF^{V600E} mutant CRC PDX model CR004.

FIG. 13 shows a graph of tumor volume over a period of treatment time with the compound of Formula I alone, encorafenib alone, and the combination of the compound of Formula I and encorafenib in BRAF^{V600E} mutant CRC CDX model WiDr.

FIG. 14 shows a graph of tumor volume over a period of treatment time with the compound of Formula I alone, encorafenib alone, and the combination of the compound of Formula I and encorafenib in BRAF^{V600E} mutant CRC CDX model HT-29.

FIG. 15 shows a graph of tumor volume over a period of treatment time with the compound of Formula I alone, encorafenib alone, and the combination of the compound of Formula I and encorafenib in BRAF^{V600E} mutant thyroid carcinoma CDX model BHT-101.

FIG. 16 shows a graph of tumor volume over a period of treatment time with the compound of Formula I alone, encorafenib alone, and the combination of the compound of Formula I and encorafenib in BRAF^{V600E} mutant CRC CDX model RKO.

FIG. 17A shows synergy data in NCI-H508 cancer cell line using the combination of the compound of Formula I and trametinib.

FIG. 17B shows synergy data in NCI-H508 cancer cell line using the combination of the compound of Formula I and binimetinib.

FIG. 17C graphic synergy data in NCI-H1666 cancer cell line using the combination of the compound of Formula I and trametinib.

FIG. 17D shows synergy data in NCI-H1666 cancer cell line using the combination of the compound of Formula I and binimetinib.

FIG. 18A shows synergy data in MeWo cancer cell line using the combination of the compound of Formula I and trametinib.

FIG. 18B shows synergy data in MeWo cancer cell line using the combination of the compound of Formula I and binimetinib.

FIG. 18C shows synergy data in NCI-H1838 cancer cell line using the combination of the compound of Formula I and trametinib.

FIG. 18D shows synergy data in NCI-H1838 cancer cell line using the combination of the compound of Formula I and binimetinib.

FIG. 19A shows a plot of percent activity versus inhibitor concentration (log M) in NCI-H508 cells treated with the compound of Formula I alone and in combination with binimetinib. The tabulated IC₅₀ data in NCI-H508 cells treated with the compound of Formula I alone and in combination with binimetinib.

FIG. 19B shows a plot of percent activity versus inhibitor concentration (log M) in MeWo cells treated with the compound of Formula I alone and in combination with binimetinib. Tabulated IC50 data in MeWo cells treated with the compound of Formula I alone and in combination with binimetinib.

FIG. 20A shows a Western blot gel indicating the synergistic inhibition of ERK1/2 phosphorylation in the NCI-H508 cancer cell line.

FIG. 20B shows a bar graph quantitation of the Western blot of FIG. 20A.

FIG. 20C shows a Western blot gel indicating the synergistic inhibition of ERK1/2 phosphorylation in the MeWo (NF1 LoF) cancer cell line.

FIG. 20D shows a bar graph quantitation of the Western blot of FIG. 20C.

FIG. 21A shows synergy data in NCI-H2009 (KRAS G12A) cancer cell line using the combination of the compound of Formula I and trametinib.

FIG. 21B shows synergy data in LS513 (KRAS G12D) cancer cell line using the combination of the compound of Formula I and trametinib.

FIG. 21C shows synergy data in A549 (KRAS G12S) cancer cell line using the combination of the compound of Formula I and trametinib.

FIG. 21D shows synergy data in NCI-H727 (KRAS G12V) cancer cell line using the combination of the compound of Formula I and trametinib.

FIG. 22A shows synergy data in NCI-H2009 (KRAS G12A) cancer cell line using the combination of the compound of Formula I and binimetinib.

FIG. 22B shows synergy data in LS513 (KRAS G12D) cancer cell line using the combination of the compound of Formula I and binimetinib.

FIG. 22C shows synergy data in A549 (KRAS G12S) cancer cell line using the combination of the compound of Formula I and binimetinib.

FIG. 22D shows synergy data in NCI-H727 (KRAS G12V) cancer cell line using the combination of the compound of Formula I and binimetinib.

FIG. 23A shows a plot of percent activity versus inhibitor concentration (log M) in LS513 (KRAS G12D) cells treated with the compound of Formula I alone and in combination with trametinib.

FIG. 23B shows a plot of percent activity versus inhibitor concentration (log M) in NCI-H2009 (KRAS G12D) cells treated with the compound of Formula I alone and in combination with trametinib. The tabulated data in NCI-H508 cells treated with the compound of Formula I alone and in combination with trametinib.

FIG. 23C shows a bar graph of percent CTG activity that indicates Formula I or trametinib alone has minimal effect on cell viability. Collectively, this data indicates that combination of the compound of Formula I and inhibitors of MEK provides synergistic inhibition of cancer cell viability in BRAF class III, NF1 LoF and KRAS G12X mutated cancer.

FIG. 24 shows a graph of tumor volume over a period of treatment time with the compound of Formula I alone, trametinib alone, and the combination of the compound of Formula I and trametinib in NF1 LoF Mutant Melanoma CDX Model MeWo.

FIG. 25 shows a graph of tumor volume over a period of treatment time with the compound of Formula I alone, binimetinib alone, and the combination of the compound of Formula I and binimetinib in NF1 LoF Mutant Melanoma CDX Model MeWo.

FIG. 26 shows a graph of tumor volume over a period of treatment time with the compound of Formula I alone, trametinib alone, and the combination of the compound of Formula I and trametinib in BRAF Class III Mutant CRC CDX Model NCI-H508.

FIG. 27 shows a graph of tumor volume over a period of treatment time with the compound of Formula I alone, trametinib alone, and the combination of the compound of Formula I and trametinib in NF1 LoF Mutant NSCLC CDX Model NCI-H1838.

FIG. 28A shows synergy data in Hs746T cancer cell line using the combination of the compound of Formula I and crizotinib.

FIG. 28B shows synergy data in MKN-45 cancer cell line using the combination of the compound of Formula I and crizotinib.

FIG. 28C shows synergy data in EBC-1 cancer cell line using the combination of the compound of Formula I and crizotinib.

FIG. 29 shows a graph of tumor volume over a period of treatment time with the compound of Formula I alone, crizotinib alone, and the combination of the compound of Formula I and crizotinib in c-MET amplified gastric cancer CDX model SNU-5.

FIG. 30 shows a graph of tumor volume over a period of treatment time with the compound of Formula I alone, crizotinib alone, and the combination of the compound of Formula I and crizotinib in c-MET amplified NSCLC CDX model NCI-H1993.

### DETAILED DESCRIPTION OF THE INVENTION

### I. General

The present disclosure provides methods of treating a subject having cancer comprising administering to the subject a therapeutically effective amount of a compound of Formula I or its pharmaceutically acceptable salt:

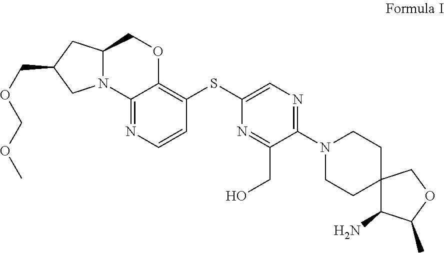 [SMILES: COCOC[C@H]1C[C@H]2COc3c(Sc4cnc(N5CCC6(CC5)CO[C@@H](C)[C@H]6N)c(CO)n4)ccnc3N2C1]

in combination with an FGFR inhibitor. The Examples below indicate a synergy for the combination that was unexpected. The combination therapies disclosed herein, employing the compound of Formula I or its pharmaceutically acceptable salt, can exhibit superior results compared to combinations of alternative SHP2 inhibitors used in combination with inhibitors of FGFR. Moreover, the combinations of the SHP2 inhibitor of Formula I and inhibitors of FGFR provide methods that allow the use of lower dosages of either agent used alone in a monotherapy, which can aid in reducing potential side effects. In particular, the combination therapies can be effective in cancer cells that express mutations including, but not limited to FGFR4 mutations, as well as amplified expression of FGFR.

In a first aspect, the present disclosure provides a method of treating a subject having cancer comprising administering to the subject a therapeutically effective amount of a compound of Formula I or its pharmaceutically acceptable salt:

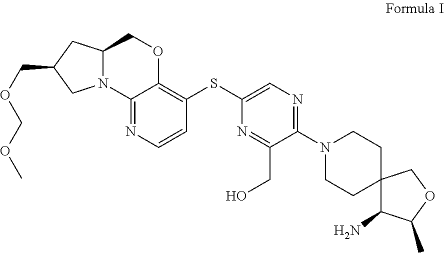 [SMILES: COCOC[C@H]1C[C@H]2COc3c(Sc4cnc(N5CCC6(CC5)CO[C@@H](C)[C@H]6N)c(CO)n4)ccnc3N2C1]

in combination with an FGFR inhibitor. In some embodiments, the FGFR in the subject is constitutively active. In some embodiments, the cancer lung cancer. In some embodiments, the cancer is hepatocellular carcinoma. In some embodiments, the cancer is cholangiocarcinoma. In some embodiments, the cancer is pancreatic ductal adenocarcinoma (PDAC). In some embodiments, the inhibitor is selected from the group consisting of erdafitinib, AZD4547, Ly2874455, CH5183284, NVP-BGJ398, INCB054828, rogaratinib, PRN1371, TAS-120, BLU-554, H3B-6527, andFGF401. In some embodiments, the FGFR inhibitor is erdafitinib. In some embodiments, the FGFR inhibitor is pemigatinib, infigratinib, dovitinib, ponatinib, nintedanib, and fisogatinib. In some embodiments, the method comprises administering a third MAPK pathway inhibitor. In some embodiments, the administration is oral. In some embodiments, the dosing of the compound of Formula I is in a range from 20 mg to 400 mg daily. In some embodiments, the dosing of the FGFR inhibitor is in a range from 1 mg to 500 mg daily.

In a second aspect, the present disclosure provides a method of treating liver cancer in a subject comprising orally administering to the subject a therapeutically effective amount of a compound of Formula I or its pharmaceutically acceptable salt:

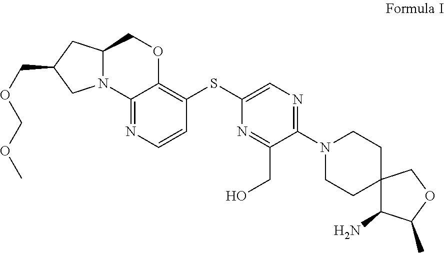 [SMILES: COCOC[C@H]1C[C@H]2COc3c(Sc4cnc(N5CCC6(CC5)CO[C@@H](C)[C@H]6N)c(CO)n4)ccnc3N2C1]

in combination with erdafitinib. In some embodiments, the compound of Formula I is administered once or twice daily. In some embodiments, erdafitinib is administered once or twice daily. In some embodiments, the subject is a human.

In a third aspect, the present disclosure provides a kit comprising a compound of Formula I or a pharmaceutically acceptable salt thereof and an FGFR inhibitor. In some embodiments, the compound of Formula I and the FGFR inhibitor are in separate packages. In some embodiments, the kit further comprises instructions to administer the contents of the kit to a subject for the treatment of cancer. In some embodiments, the FGFR inhibitor is one or more of erdafitinib, AZD4547, Ly2874455, CH5183284, NVP-BGJ398, INCB054828, rogaratinib, PRN1371, TAS-120, BLU-554, H3B-6527, FGF401, pemigatinib, infigratinib, dovitinib, ponatinib, nintedanib, and fisogatinib.

In a fourth aspect, the present disclosure provides a method of treating a subject having cancer comprising administering to the subject a therapeutically effective amount of a compound of Formula I or its pharmaceutically acceptable salt:

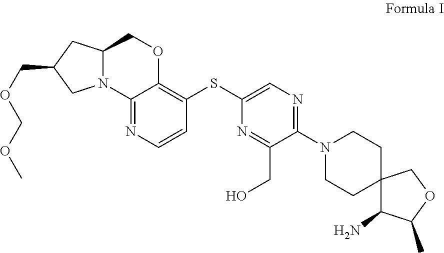 [SMILES: COCOC[C@H]1C[C@H]2COc3c(Sc4cnc(N5CCC6(CC5)CO[C@@H](C)[C@H]6N)c(CO)n4)ccnc3N2C1]

in combination with an inhibitor of a B-Raf protein having a class 1 mutation. In some embodiments, the class 1 mutation is V600E. In some embodiments, the cancer is colorectal cancer. In some embodiments, the cancer is melanoma. In some embodiments, the cancer is thyroid cancer. In some embodiments, the cancer is pancreatic ductal adenocarcinoma (PDAC). In some embodiments, the inhibitor is selected from the group consisting of encorafenib, vemurafenib, dabrafenib, sorafenib, and regorafenib. In some embodiments, the inhibitor is encorafenib. In some embodiments, the inhibitor is vemurafenib. In some embodiments, the inhibitor is dabrafenib. In some embodiments, the inhibitor is sorafenib. In some embodiments, the inhibitor is regorafenib. In some embodiments, the method comprises administering a third MAPK pathway inhibitor. In some embodiments, the administration is oral. In some embodiments, the dosing of the compound of Formula I is in a range from 20 mg to 400 mg daily. In some embodiments, the dosing of the B-Raf inhibitor is in a range from 1 mg to 500 mg.

The present disclosure provides methods of treating a subject having cancer comprising administering to the subject a therapeutically effective amount of a compound of Formula I or its pharmaceutically acceptable salt:

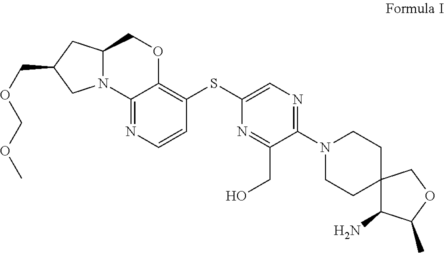 [SMILES: COCOC[C@H]1C[C@H]2COc3c(Sc4cnc(N5CCC6(CC5)CO[C@@H](C)[C@H]6N)c(CO)n4)ccnc3N2C1]

in combination with an inhibitor of a class 1 mutant B-Raf. The Examples below indicate a significant synergy for the combination that was unexpected. The combination therapies disclosed herein, employing the compound of Formula I or its pharmaceutically acceptable salt, can exhibit superior results compared to combinations of alternative SHP2 inhibitors used in combination with inhibitors of class 1 mutant B-Raf. Moreover, the combinations of the SHP2 inhibitor of Formula I and inhibitors of class 1 mutant B-Raf provide methods that allow the use of lower dosages of either agent used alone in a monotherapy, which can aid in reducing potential side effects. In particular, the combination therapies can be effective in cancer cells that express the BRAF V600E mutation.

In a fourth aspect, the present disclosure provides a method of treating a subject having cancer comprising administering to the subject a therapeutically effective amount of a compound of Formula I or its pharmaceutically acceptable salt:

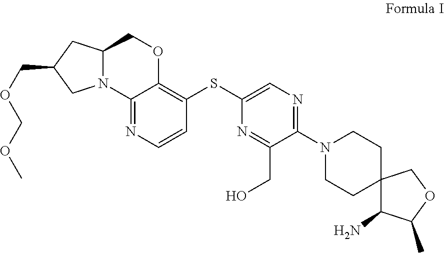 [SMILES: COCOC[C@H]1C[C@H]2COc3c(Sc4cnc(N5CCC6(CC5)CO[C@@H](C)[C@H]6N)c(CO)n4)ccnc3N2C1]

in combination with an inhibitor of a B-Raf protein having a class 1 mutation. In some embodiments, the class 1 mutation is V600E. In some embodiments, the cancer is colorectal cancer. In some embodiments, the cancer is melanoma. In some embodiments, the cancer is thyroid cancer. In some embodiments, the cancer is pancreatic ductal adenocarcinoma (PDAC). In some embodiments, the inhibitor is selected from the group consisting of encorafenib, vemurafenib, dabrafenib, sorafenib, and regorafenib. In some embodiments, the inhibitor is encorafenib. In some embodiments, the inhibitor is vemurafenib. In some embodiments, the inhibitor is dabrafenib. In some embodiments, the inhibitor is sorafenib. In some embodiments, the inhibitor is regorafenib. In some embodiments, the method comprises administering a third MAPK pathway inhibitor. In some embodiments, the administration is oral. In some embodiments, the dosing of the compound of Formula I is in a range from 20 mg to 400 mg daily. In some embodiments, the dosing of the B-Raf inhibitor is in a range from 1 mg to 500 mg.

In another aspect, the present disclosure provides a method of treating colorectal cancer in a subject comprising orally administering to the subject a therapeutically effective amount of a compound of Formula I or its pharmaceutically acceptable salt:

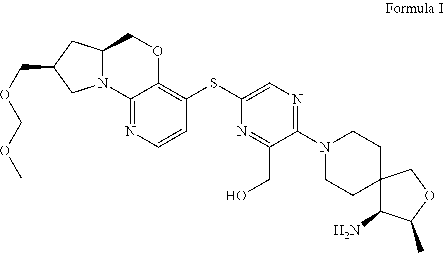 [SMILES: COCOC[C@H]1C[C@H]2COc3c(Sc4cnc(N5CCC6(CC5)CO[C@@H](C)[C@H]6N)c(CO)n4)ccnc3N2C1]

in combination with B-Raf inhibitor encorafenib. In some embodiments, the compound of Formula I is administered once or twice daily. In some embodiments, encorafenib is administered once or twice daily. In some embodiments, the subject is human.

In another aspect, the present disclosure provides a method of treating a subject having cancer comprising administering to the subject a therapeutically effective amount of a compound of Formula I or its pharmaceutically acceptable salt:

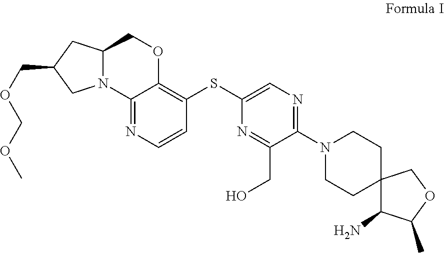 [SMILES: COCOC[C@H]1C[C@H]2COc3c(Sc4cnc(N5CCC6(CC5)CO[C@@H](C)[C@H]6N)c(CO)n4)ccnc3N2C1]

in combination with encorafenib.

In another aspect, the present disclosure provides a method of treating a subject having cancer comprising administering to the subject a therapeutically effective amount of a compound of Formula I or its pharmaceutically acceptable salt:

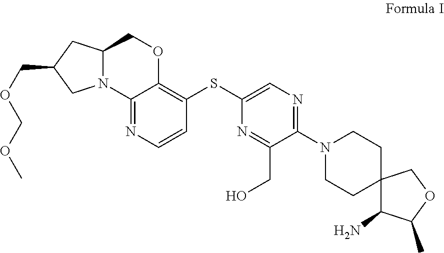 [SMILES: COCOC[C@H]1C[C@H]2COc3c(Sc4cnc(N5CCC6(CC5)CO[C@@H](C)[C@H]6N)c(CO)n4)ccnc3N2C1]

in combination with vemurafenib

In another aspect, the present disclosure provides a method of treating a subject having cancer comprising administering to the subject a therapeutically effective amount of a compound of Formula I or its pharmaceutically acceptable salt:

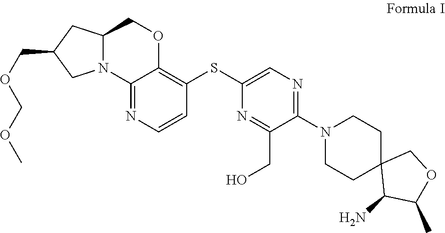 [SMILES: COCOC[C@H]1C[C@H]2COc3c(Sc4cnc(N5CCC6(CC5)CO[C@@H](C)[C@H]6N)c(CO)n4)ccnc3N2C1]

in combination with dabrafenib

In another aspect, the present disclosure provides a method of treating a subject having cancer comprising administering to the subject a therapeutically effective amount of a compound of Formula I or its pharmaceutically acceptable salt:

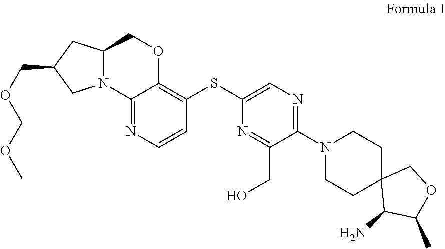 [SMILES: COCOC[C@H]1C[C@H]2COc3c(Sc4cnc(N5CCC6(CC5)CO[C@@H](C)[C@H]6N)c(CO)n4)ccnc3N2C1]

in combination with sorafenib.

In another aspect, the present disclosure provides a method of treating a subject having cancer comprising administering to the subject a therapeutically effective amount of a compound of Formula I or its pharmaceutically acceptable salt:

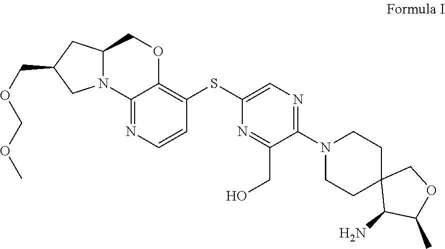 [SMILES: COCOC[C@H]1C[C@H]2COc3c(Sc4cnc(N5CCC6(CC5)CO[C@@H](C)[C@H]6N)c(CO)n4)ccnc3N2C1]

in combination with regorafenib.

In various embodiments of the methods described herein, the cancer is colorectal cancer. In some embodiments, the cancer is thyroid cancer. In some embodiments, the cancer is melanoma. In some embodiments, the cancer is pancreatic ductal adenocarcinoma (PDAC). In some embodiments, a dosing of the B-Raf inhibitor is less than a dosing required for a monotherapy with the B-Raf inhibitor. In some embodiments, a dosing of the compound of Formula I is less than a dosing required for a monotherapy with the compound of Formula I.

In another aspect, the present disclosure provides a method of inhibiting ERK1/2 phosphorylation in a cell population comprising contacting a cell population with the compound of Formula I or its pharmaceutically acceptable salt:

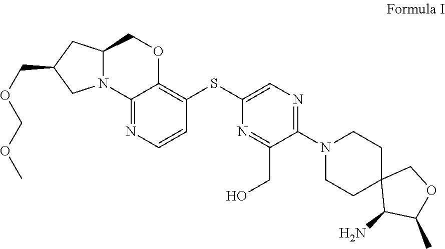 [SMILES: COCOC[C@H]1C[C@H]2COc3c(Sc4cnc(N5CCC6(CC5)CO[C@@H](C)[C@H]6N)c(CO)n4)ccnc3N2C1]

in combination with regorafenib. In some embodiments, a concentration of the compound of Formula I is a range from 1 nM to 500 nM. In some embodiments, a concentration of encorafenib is in a range from 10 nM to 20 nM.

In another aspect, the present disclosure provides a kit comprising a compound of Formula I or a pharmaceutically acceptable salt thereof and a B-Raf inhibitor. In some embodiments, the compound of Formula I and the B-Raf inhibitors are in separate packages. In some embodiments, the kit further comprises instructions to administer the contents of the kit to a subject for the treatment of cancer. In some embodiments, the B-Raf inhibitor is one or more of encorafenib, vemurafenib, dabrafenib, sorafenib, and regorafenib.

The present embodiments provide methods of treating a subject having cancer comprising administering to the subject a therapeutically effective amount of a compound of Formula I or its pharmaceutically acceptable salt:

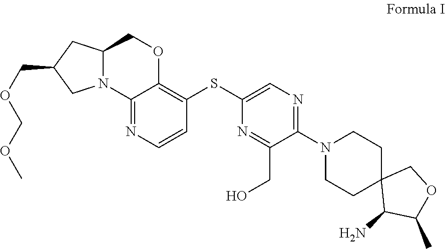 [SMILES: COCOC[C@H]1C[C@H]2COc3c(Sc4cnc(N5CCC6(CC5)CO[C@@H](C)[C@H]6N)c(CO)n4)ccnc3N2C1]

in combination with an a MEK inhibitor. The Examples below indicate a synergy for the combination that was unexpected. The combination therapies disclosed herein, employing the compound of Formula I or its pharmaceutically acceptable salt, can exhibit superior results compared to combinations of alternative SHP2 inhibitors used in combination with inhibitors of MEK. Moreover, the combinations of the SIP2 inhibitor of Formula I and inhibitors of MEK provide methods that allow the use of lower dosages of either agent used alone in a monotherapy, which can aid in reducing potential side effects. In particular, the combination therapies can be effective in cancer cells that express mutations including, but not limited to class III B-raf mutations and KRAS G12X mutations.

In another aspect, the present disclosure provides a method of treating a subject having cancer comprising administering to the subject a therapeutically effective amount of a compound of Formula I or its pharmaceutically acceptable salt:

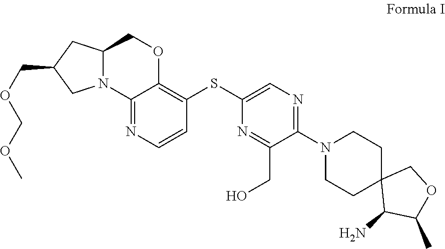 [SMILES: COCOC[C@H]1C[C@H]2COc3c(Sc4cnc(N5CCC6(CC5)CO[C@@H](C)[C@H]6N)c(CO)n4)ccnc3N2C1]

in combination with an a MEK inhibitor. In some embodiments, the MEK inhibitor inhibits MEK1 selectively or MEK2 selectively or both MEK1 and MEK2 selectively. In some embodiments, the cancer is metastatic. In some embodiments, the cancer colorectal cancer. In some embodiments, the cancer is melanoma. In some embodiments, the cancer is lung cancer. In some embodiments, the cancer is pancreatic cancer. In some embodiments, the cancer is breast cancer. In some embodiments, the cancer is pancreatic ductal adenocarcinoma (PDAC). In some embodiments, the MEK inhibitor is selected from the group consisting of trametinib, cobimetinib, binimetinib, PD-0325901, selumetinib and CI-1040. In some embodiments, the MEK inhibitor is trametinib. In some embodiments, the MEK inhibitor is cobimetinib. In some embodiments, the MEK inhibitor is binimetinib. In some embodiments, the MEK inhibitor is PD-325901. In some embodiments, the MEK inhibitor is CI-1040. In some embodiments, the method comprises administering a further MAPK pathway inhibitor. In some embodiments, the administration is oral. In some embodiments, the dosing of the compound of Formula I is in a range from 20 mg to 400 mg daily. In some embodiments, the dosing of the MEK inhibitor is in a range from 1 mg to 500 mg daily.

In another aspect, the present disclosure provides a method of treating cancer in a subject comprising orally administering to the subject a therapeutically effective amount of a compound of Formula I or its pharmaceutically acceptable salt:

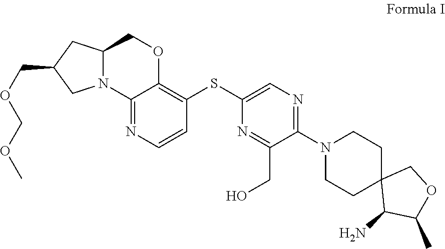 [SMILES: COCOC[C@H]1C[C@H]2COc3c(Sc4cnc(N5CCC6(CC5)CO[C@@H](C)[C@H]6N)c(CO)n4)ccnc3N2C1]

in combination with MEK inhibitor binimetinib or trametinib. In some embodiments, the compound of Formula I is administered once or twice daily. In some embodiments, binimetinib or trametinib is administered once or twice daily. In some embodiments, the subject is a human.

In another aspect, the present disclosure provides a method of treating a subject having cancer comprising administering to the subject a therapeutically effective amount of a compound of Formula I or its pharmaceutically acceptable salt:

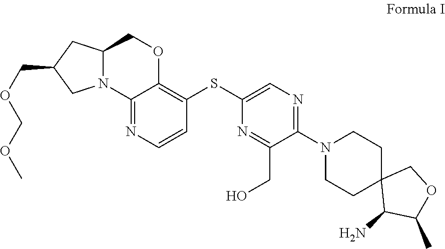 [SMILES: COCOC[C@H]1C[C@H]2COc3c(Sc4cnc(N5CCC6(CC5)CO[C@@H](C)[C@H]6N)c(CO)n4)ccnc3N2C1]

in combination with binimetinib.

In another aspect, the present disclosure provides a method of treating a subject having cancer comprising administering to the subject a therapeutically effective amount of a compound of Formula I or its pharmaceutically acceptable salt:

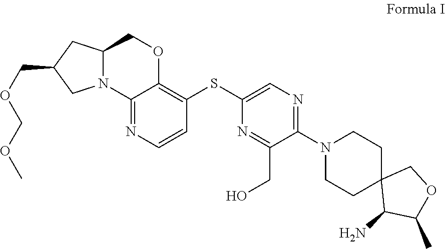 [SMILES: COCOC[C@H]1C[C@H]2COc3c(Sc4cnc(N5CCC6(CC5)CO[C@@H](C)[C@H]6N)c(CO)n4)ccnc3N2C1]

in combination with trametinib.

In various embodiments of the methods described herein, the cancer is colorectal cancer. In some embodiments, the cancer is lung cancer. In some embodiments, the cancer is melanoma. In some embodiments, the cancer is pancreatic ductal adenocarcinoma (PDAC). In some embodiments, a dosing of the MEK inhibitor is less than a dosing required for a monotherapy with the MEK inhibitor. In some embodiments, a dosing of the compound of Formula I is less than a dosing required for a monotherapy with the compound of Formula I.

In another aspect, the present disclosure provides a method of inhibiting ERK1/2 phosphorylation comprising contacting a cell population with Formula I or its pharmaceutically acceptable salt:

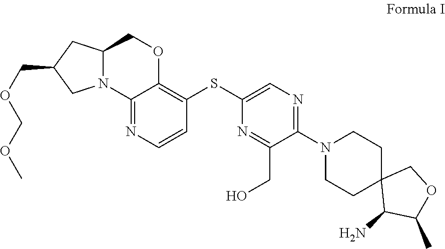 [SMILES: COCOC[C@H]1C[C@H]2COc3c(Sc4cnc(N5CCC6(CC5)CO[C@@H](C)[C@H]6N)c(CO)n4)ccnc3N2C1]

in combination with binimetinib or trametinib. In some embodiments, a concentration of the compound of Formula I is in a range from 1 nM to 1,000 nM. In some embodiments, a concentration of MEK inhibitors is in a range from 10 nM to 500 nM.

In another aspect, the present disclosure provides a kit comprising a compound of Formula I or a pharmaceutically acceptable salt thereof and an MEK inhibitor. In some embodiments, the compound of Formula I and the MEK inhibitor are in separate packages. In some embodiments, the kit further comprises instructions to administer the contents of the kit to a subject for the treatment of cancer. In some embodiments, the MEK inhibitor is one or more of trametinib or binimetinib.

The present disclosure provides methods of treating a subject having cancer comprising administering to the subject a therapeutically effective amount of a compound of Formula I or its pharmaceutically acceptable salt:

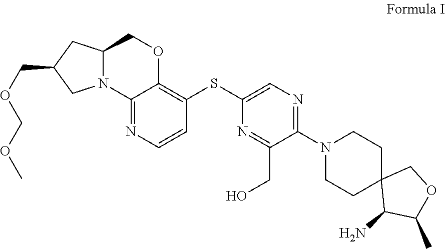 [SMILES: COCOC[C@H]1C[C@H]2COc3c(Sc4cnc(N5CCC6(CC5)CO[C@@H](C)[C@H]6N)c(CO)n4)ccnc3N2C1]

in combination with a MET inhibitor. The Examples below indicate a synergy for the combination that was unexpected. The combination therapies disclosed herein, employing the compound of Formula I or its pharmaceutically acceptable salt, can exhibit superior results compared to combinations of alternative SHP2 inhibitors used in combination with inhibitors of MET. Moreover, the combinations of the SHP2 inhibitor of Formula I and inhibitors of MET provide methods that allow the use of lower dosages of either agent used alone in a monotherapy, which can aid in reducing potential side effects. In particular, the combination therapies can be effective in cancer cells that express aberrant mutations in MET.

In another aspect, the present disclosure provides a method of treating a subject having cancer comprising administering to the subject a therapeutically effective amount of a compound of Formula I or its pharmaceutically acceptable salt:

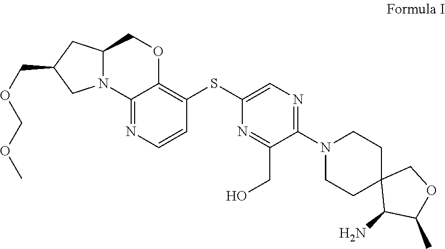 [SMILES: COCOC[C@H]1C[C@H]2COc3c(Sc4cnc(N5CCC6(CC5)CO[C@@H](C)[C@H]6N)c(CO)n4)ccnc3N2C1]

in combination with a MET inhibitor. In some embodiments, the MET inhibitor is also an ALK inhibitor, a ROS1 inhibitor, or both. In some embodiments, the cancer is non-small lung cancer. In some embodiments, the cancer is stomach cancer. In some embodiments, the cancer is gastric adenocarcinoma. In some embodiments, the cancer is pancreatic ductal adenocarcinoma (PDAC). In some embodiments, the MET inhibitor is selected from the group consisting of crizotinib, tepotinib, savolitinib, cabozantinib, and tivantinib. In some embodiments, the MET inhibitor is crizotinib. In some embodiments, the MET inhibitor is tepotinib. In some embodiments, the inhibitor is savolitinib. In some embodiments, the inhibitor is cabozantinib. In some embodiments, the inhibitor is tivantinib. In some embodiments, the method comprises administering a third MAPK pathway inhibitor. In some embodiments, the administration is oral. In some embodiments, the dosing of the compound of Formula I is in a range from 10 mg to 500 mg daily. In some embodiments, the dosing of the inhibitor is in a range from 20 mg to 400 mg daily.

In another aspect, the present disclosure provides a method of treating stomach cancer in a subject comprising orally administering to the subject a therapeutically effective amount of a compound of Formula I or its pharmaceutically acceptable salt:

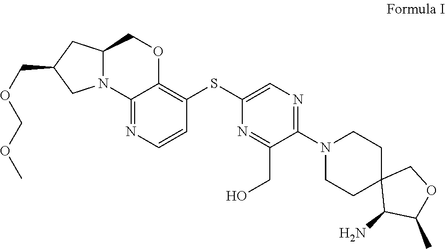 [SMILES: COCOC[C@H]1C[C@H]2COc3c(Sc4cnc(N5CCC6(CC5)CO[C@@H](C)[C@H]6N)c(CO)n4)ccnc3N2C1]

in combination with crizotinib. In some embodiments, the compound of Formula I is administered once or twice daily. In some embodiments, crizotinib is administered once or twice daily. In some embodiments, the subject is a human.

In a final aspect, the present disclosure provides a kit comprising a compound of Formula I or a pharmaceutically acceptable salt thereof and a MET inhibitor. In some embodiments, the compound of Formula I and the MET inhibitor are in separate packages. In some embodiments, the kit further comprises instructions to administer the contents of the kit to a subject for the treatment of cancer. In some embodiments, the MET inhibitor is one or more of crizotinib, tepotinib, savolitinib, cabozantinib, and tivantinib.

Accordingly, such treatments comport with the use of companion diagnostics to aid in proper patient population selection. These and other advantages will be recognized by those skilled in the art.

### II. Definitions

Unless specifically indicated otherwise, all technical and scientific terms used herein have the same meaning as commonly understood by those of ordinary skill in the art to which the embodiments are directed. In addition, any method or material similar or equivalent to a method or material described herein can be used in the practice of the embodiments herein. For purposes of the embodiments disclosed herein, the following terms are defined.

“A,” “an,” or “the” as used herein not only include aspects with one member, but also include aspects with more than one member. For instance, the singular forms “a,” “an,” and “the” include plural referents unless the context clearly dictates otherwise. Thus, for example, reference to “a cell” includes a plurality of such cells and reference to “the agent” includes reference to one or more agents known to those skilled in the art, and so forth.

“Pharmaceutically acceptable excipient” refers to a substance that aids the administration of an active agent to and absorption by a subject. Pharmaceutical excipients useful in the present embodiments include, but are not limited to, binders, fillers, disintegrants, lubricants, surfactants, coatings, sweeteners, flavors and colors. One of skill in the art will recognize that other pharmaceutical excipients are useful in the present embodiments.

“Treat”, “treating” and “treatment” refer to any indicia of success in the treatment or amelioration of an injury, pathology or condition, including any objective or subjective parameter such as abatement; remission; diminishing of symptoms or making the injury, pathology or condition more tolerable to the patient; slowing in the rate of degeneration or decline; making the final point of degeneration less debilitating; improving a patient's physical or mental well-being. The treatment or amelioration of symptoms can be based on objective or subjective parameters; including the results of a physical examination, neuropsychiatric exams, and/or a psychiatric evaluation.

“Administering” refers to oral administration, administration as a suppository, topical contact, parenteral, intravenous, intraperitoneal, intramuscular, intralesional, intranasal or subcutaneous administration, intrathecal administration, or the implantation of a slow-release device e.g., a mini-osmotic pump, to the subject. In the context of the combination therapies disclosed herein, administration can be at separate times or simultaneous or substantially simultaneous.

“Therapeutically effective amount” refers to a dose that produces therapeutic effects for which it is administered. The exact dose will depend on the purpose of the treatment, and will be ascertainable by one skilled in the art using known techniques (see, e.g., Lieberman, Pharmaceutical Dosage Forms (vols. 1-3, 1992); Lloyd, The Art, Science and Technology of Pharmaceutical Compounding (1999); Pickar, Dosage Calculations (1999); and Remington: The Science and Practice of Pharmacy, 20th Edition, 2003, Gennaro, Ed., Lippincott, Williams & Wilkins). In sensitized cells, the therapeutically effective dose can often be lower than the conventional therapeutically effective dose for non-sensitized cells.

“Inhibition,” “inhibits” and “inhibitor” refer to a compound that partially or completely blocks or prohibits or a method of partially or fully blocking or prohibiting, a specific action or function.

“Cancer” refers to all types of cancer, neoplasm or malignant tumors found in mammals (e.g. humans), including, without limitation, leukemias, lymphomas, carcinomas and sarcomas. Exemplary cancers that may be treated with a compound or method provided herein include brain cancer, glioma, glioblastoma, neuroblastoma, prostate cancer, colorectal cancer, pancreatic cancer, medulloblastoma, melanoma, cervical cancer, gastric cancer, ovarian cancer, lung cancer, cancer of the head, Hodgkin's Disease, and Non-Hodgkin's Lymphomas. Exemplary cancers that may be treated with a compound or method provided herein include cancer of the thyroid, endocrine system, brain, breast, cervix, colon, head & neck, liver, kidney, lung, ovary, pancreas, rectum, stomach, and uterus. Additional examples include, thyroid carcinoma, cholangiocarcinoma, pancreatic adenocarcinoma, skin cutaneous melanoma, colon adenocarcinoma, rectum adenocarcinoma, stomach adenocarcinoma, esophageal carcinoma, head and neck squamous cell carcinoma, breast invasive carcinoma, lung adenocarcinoma, lung squamous cell carcinoma, non-small cell lung carcinoma, mesothelioma, multiple myeloma, neuroblastoma, glioma, glioblastomamultiforme, ovarian cancer, rhabdomyosarcoma, primary thrombocytosis, primary macroglobulinemia, primary brain tumors, malignant pancreatic insulanoma, malignant carcinoid, urinary bladder cancer, premalignant skin lesions, testicular cancer, thyroid cancer, neuroblastoma, esophageal cancer, genitourinary tract cancer, malignant hypercalcemia, endometrial cancer, adrenal cortical cancer, neoplasms of the endocrine or exocrine pancreas, medullary thyroid cancer, medullary thyroid carcinoma, melanoma, colorectal cancer, papillary thyroid cancer, hepatocellular carcinoma, pancreatic ductal adenocarcinoma (PDAC), or prostate cancer.

“FGFR inhibitor” refers to any inhibitor of wild-type FGFR or an FGFR mutant. FGFR mutations include, without limitation, single nucleotide polymorphisms, exon insertion and deletions, polysomy, and the like. Specific examples of mutations and inhibitors include, without limitation, FGFR1 gene copy gain, FGFR1 gene amplification, FGFR2 gene copy gain, FGFR2 gene amplification, FGFR3 gene copy gain, FGFR3 gene amplification, FGFR4 gene copy gain, FGFR4 gene amplification, FGFR1 T141R, FGFR1 R445W, FGFR1 N546K, FGFR1 K656E, FGFR1 G818R, FGFR2 S252W, FGFR2 P253R, FGFR2 A315T, FGFR2 D336N, FGFR2 Y375C, FGFR2 C382R, FGFR2 V395D, FGFR2 D471N, FGFR2 I547V, FGFR2 N549K, FGFR2 N549Y, FGFR2 K659E, FGFR3 S131L, FGFR3 R248C, FGFR3 S249C, FGFR3 G370C, FGFR3 S371C, FGFR3 Y373C, FGFR3 G380R, FGFR3 R399C, FGFR3 E627K, FGFR3 K650E, FGFR3 K650M, FGFR3 V677I, FGFR3 D785Y, FGFR4 R183S, FGFR4 R394Q, FGFR4 D425N, FGFR4 V510L, FGFR4 R610H, and FGFR fusions (e.g., FGFR3-TACC3, FGFR2-TACC3, FGFR2-NPM1, FGFR2-TACC2, FGFR2-BICC1, FGFR2-C10orf68, FGFR3-JAKMIP1, FGFR2-KIAA1598, FGFR2-NCALD, FGFR2-NOL4, FGFR1-NTM, FGFR2-PPAPDC1A, FGFR3-TNIP2, and FGFR3-WHSC1). In some embodiments, one or more of the above-listed mutated forms can be specifically excluded from the embodiments set forth herein, including without limitation, any methods, kits and compositions of matter, etc. Inhibitors include, without limitation, erdafitinib, pemigatinib, infigratinib, dovitinib, ponatinib, nintedanib, and fisogatinib. In some embodiments, one or more of the above-listed mutated forms can be specifically excluded from the embodiments set forth herein, including without limitation, any methods, kits and compositions of matter, etc.

Class 1 mutant B-Raf” or “B-Raf protein having a class 1 mutation” refers generally to any mutation that deviates from the wildtype B-Raf protein at V600 (valine 600). In particular, such mutant B-Raf proteins include mutations include the V600E mutation. Other class 1 BRAF mutations include, without limitation, V600K, V600D, V600L, V600M and V600R. In some embodiments, one or more of the above-listed mutations can be specifically excluded from the embodiments set forth herein, including without limitation, any methods, kits and compositions of matter

“MEK inhibitor” refers generally to any inhibitor that inhibits MEK1 or MEK2 selectively or both MEK1 and MEK2. Example inhibitors include, without limitation, trametinib, cobimetinib, binimetinib, PD-0325901, selumetinib and CI-1040.

“MET inhibitor” refers to any inhibitor of wild-type MET or MET mutant. MET mutations include, without limitation, single nucleotide polymorphisms, exon insertion and deletions, polysomy, and the like. Specific examples of mutations and inhibitors include, without limitation, MET gene copy gain, MET gene amplification, MET E34K, MET H150Y, MET E168D, MET L269V, MET L299F, MET S323G, MET M362T, MET N375S, MET C385Y, MET R970C, MET R988C, MET P1009S, MET T1010I, MET S1058P, MET exon 14 skipping mutations, MET exon 14 splice variants, MET A1108S, MET V1110I, MET H1112R, MET H1112L, MET H1112I, MET HJ1124D, MET Gi 137V, MET M1149T, MET T1191I, MET V1206L, MET L1213V, MET D1228V, MET Y1230C, MET Y1230H, MET Y1230D, MET Y1235D, MET V1238I, MET D1246N, MET Y1248C, MET Y1248D, MET Y1248H, MET K1262R, MET M1268T, MET M1268I, and MET V1312I. In some embodiments, one or more of the mutated forms listed in this paragraph and elsewhere herein can be specifically excluded from the embodiments set forth herein, including without limitation, any methods, kits and compositions of matter, etc. Example inhibitors include, without limitation, crizotinib, capmatinib, tepotinib, savolitinib, tivantinib, cabozantinib, foretinib, amivantamab, onartuzumab, emibetuzumab, and ficlatuzumab. In some embodiments, one or more of the inhibitors listed in this paragraph and elsewhere herein can be specifically excluded from the embodiments set forth herein, including without limitation, any methods, kits and compositions of matter, etc.

“Subject” refers to a living organism suffering from or prone to a disease or condition that can be treated by administration of a pharmaceutical composition as provided herein. Non-limiting examples include humans, other mammals, bovines, rats, mice, dogs, monkeys, goat, sheep, cows, deer, horse, and other non-mammalian animals. In some embodiments, the patient is human.

### III. Dosing Methods

In some embodiments, the compound of Formula I, or a pharmaceutically acceptable salt thereof, is formulated as a pharmaceutical composition. In some embodiments, the compound of Formula I, or a pharmaceutically acceptable salt thereof, is formulated as an oral composition.

In some embodiments, the compound of Formula I, or a pharmaceutically acceptable salt thereof, is administered once or twice a day. In some embodiments, the compound of Formula I, or a pharmaceutically acceptable salt thereof, is administered once a day. In some embodiments, the compound of Formula I, or a pharmaceutically acceptable salt thereof, is administered twice a day. In some embodiments, the compound of Formula I, or a pharmaceutically acceptable salt thereof, is administered over a continuous 28-day cycle.

In some embodiments, the compound of Formula I, or a pharmaceutically acceptable salt thereof, is administered once a day in the amount of about 10 mg to about 140 mg.

In some embodiments, the compound of Formula I, or a pharmaceutically acceptable salt thereof, is administered once a day for a 3-week cycle, comprising 2 weeks of administration of the compound followed by 1 week of no administration of the compound.

In some embodiments, the compound of Formula I, or a pharmaceutically acceptable salt thereof, is administered once a day for a 4-week cycle, comprising 3 weeks of administration of the compound followed by 1 week of no administration of the compound.

In some embodiments, the compound of Formula I, or a pharmaceutically acceptable salt thereof, is administered over a period of 6 weeks. In some embodiments, the compound of Formula I, or a pharmaceutically acceptable salt thereof, is administered over a period of 8 weeks.

In some embodiments, the compound of Formula I, or a pharmaceutically acceptable salt thereof, is administered 3 times a week. In some embodiments, the compound of Formula I, or a pharmaceutically acceptable salt thereof, is administered on day 1, day 3, and day 5 of the week.

In some embodiments, the compound of Formula I, or a pharmaceutically acceptable salt thereof, is administered 4 times a week.

In some embodiments, the compound of Formula I, or a pharmaceutically acceptable salt thereof, is administered for a 3-week cycle, comprising 2 weeks of administration of the compound followed by 1 week of no administration of the compound.

In some embodiments, the compound of Formula I, or a pharmaceutically acceptable salt thereof, is administered for a 4-week cycle, comprising 3 weeks of administration of the compound followed by 1 week of no administration of the compound.

In some embodiments, the compound of Formula I, or a pharmaceutically acceptable salt thereof, is administered twice a day, two days per week. In some embodiments, the compound of Formula I, or a pharmaceutically acceptable salt thereof, is administered over a period of 8 weeks. In some embodiments, the compound of Formula I, or a pharmaceutically acceptable salt thereof, is administered on day 1 and day 2 of each week.

In some embodiments, the cancer is selected from lung cancer, stomach cancer, liver cancer, colon cancer, kidney cancer, breast cancer, pancreatic cancer, juvenile myelomonocytic leukemia, neurolastoma, melanoma, and acute myeloid leukemia. In some embodiments, the cancer is pancreatic ductal adenocarcinoma (PDAC).

### IV. Combination Methods

In some embodiments, the method comprises administering a third MAPK pathway inhibitor. Without being bound by theory, suppression of MAPK signaling in cancer cells can result in downregulation of PD-L1 expression and increase the likelihood that the cancer cells are detected by the immune system. Such third MAPK pathway inhibitors may be based on other mutations of proteins in the MAPK pathway. In some embodiments, any MAPK pathway inhibitor can be employed, including those targeting K-Ras, N-Ras, H-Ras, PDGFRA, PDGFRB, MET, FGFR, ALK, ROS1, TRKA, TRKB, TRKC, EGFR, IGF1R, GRB2, SOS, ARAF, BRAF, RAF1, MEK1, MEK2, c-Myc, CDK4, CDK6, CDK2, ERK1, and ERK2. Non-limiting examples of MEK inhibitors include trametinib, cobimetinib, binimetinib, PD-0325901, selumetinib and CI-1040. Exemplary MAPK pathway inhibitors include, without limitation, afatinib, osimertinib, erlotinib, gefitinib, lapatinib, neratinib, dacomitinib, vandetanib, cetuximab, panitumumab, nimotuzumab, necitumumab, trametinib, binimetinib, cobimetinib, selumetinib, ulixertinib, LTT462, and LY3214996. In some embodiments, one or more of the inhibitors listed in this paragraph and elsewhere herein, can be specifically excluded from one or more of the embodiments set forth herein, including without limitation, any methods, kits and compositions of matter, etc.

The methods disclosed herein can be combined with other chemotherapeutic agents. Examples of such agents can be found in Cancer Principles and Practice of Oncology by V. T. Devita and S. Hellman (editors), 6th edition (Feb. 15, 2001), Lippincott Williams & Wilkins Publishers; which is incorporated herein by reference in its entirety for all of its teachings, including without limitation all methods, compounds, compositions, data and the like, for use with any of the embodiments and disclosure herein. A person of ordinary skill in the art would be able to discern which combinations of agents would be useful based on the particular characteristics of the drugs and the disease involved.

In some embodiments, the methods can include the co-administration of at least one cytotoxic agent. The term “cytotoxic agent” as used herein refers to a substance that inhibits or prevents a cellular function and/or causes cell death or destruction. Cytotoxic agents include, but are not limited to, radioactive isotopes (e.g., At211, I131, I125, Y90, Re186, Re188, Sm153, Bi212, P32, Pb212 and radioactive isotopes of Lu); chemotherapeutic agents; growth inhibitory agents; enzymes and fragments thereof such as nucleolytic enzymes; and toxins such as small molecule toxins or enzymatically active toxins of bacterial, fungal, plant or animal origin, including fragments and/or variants thereof.

Examples of cytotoxic agents can be selected from anti-microtubule agents, platinum coordination complexes, alkylating agents, antibiotic agents, topoisomerase II inhibitors, antimetabolites, topoisomerase I inhibitors, hormones and hormonal analogues, signal transduction pathway inhibitors, non-receptor tyrosine kinase angiogenesis inhibitors, immunotherapeutic agents, proapoptotic agents, inhibitors of LDH-A; inhibitors of fatty acid biosynthesis; cell cycle signaling inhibitors; HDAC inhibitors, proteasome inhibitors; and inhibitors of cancer metabolism.

Chemotherapeutic agents include chemical compounds useful in the treatment of cancer. Examples of chemotherapeutic agents include erlotinib (TARCEVA®, Genentech/OSI Pharm.), bortezomib (VELCADE®, Millennium Pharm.), disulfiram, epigallocatechin gallate, salinosporamide A, carfilzomib, 17-AAG (geldanamycin), radicicol, lactate dehydrogenase A (LDH-A), fulvestrant (FASLODEX®, AstraZeneca), sunitinib (SUTENT®, Pfizer/Sugen), letrozole (FEMARA®, Novartis), imatinib mesylate (GLEEVEC®, Novartis), finasunate (VATALANIB®, Novartis), oxaliplatin (ELOXATIN®, Sanofi), 5-FU (5-fluorouracil), leucovorin, Rapamycin (Sirolimus, RAPAMUNE®, Wyeth), Lapatinib (TYKERB®, GSK572016, Glaxo Smith Kline), lonafarnib (SCH 66336), sorafenib (NEXAVAR®, Bayer Labs), gefitinib (IRESSA®, AstraZeneca), AG1478, alkylating agents such as thiotepa and CYTOXAN® cyclosphosphamide; alkyl sulfonates such as busulfan, improsulfan and piposulfan; aziridines such as benzodopa, carboquone, meturedopa, and uredopa; ethylenimines and methylamelamines including altretamine, triethylenemelamine, triethylenephosphoramide, triethylenethiophosphoramide and trimethylomelamine; acetogenins (especially bullatacin and bullatacinone); a camptothecin (including topotecan and irinotecan); bryostatin; callystatin; CC 1065 (including its adozelesin, carzelesin and bizelesin synthetic analogs); cryptophycins (particularly cryptophycin 1 and cryptophycin 8); adrenocorticosteroids (including prednisone and prednisolone); cyproterone acetate; 5-alpha-reductases including finasteride and dutasteride); vorinostat, romidepsin, panobinostat, valproic acid, mocetinostat dolastatin; aldesleukin, talc duocarmycin (including the synthetic analogs, KW-2189 and CB1-TM1); eleutherobin; pancratistatin; a sarcodictyin; spongistatin; nitrogen mustards such as chlorambucil, chlomaphazine, chlorophosphamide, estramustine, ifosfamide, mechlorethamine, mechlorethamine oxide hydrochloride, melphalan, novembichin, phenesterine, prednimustine, trofosfamide, uracil mustard; nitrosoureas such as carmustine, chlorozotocin, fotemustine, lomustine, nimustine, and ranimnustine; antibiotics such as the enediyne antibiotics (e.g., calicheamicin, especially calicheamicin γ1I and calicheamicin ω1I (Angew Chem. Intl. Ed. Engl. 1994 33:183-186); dynemicin, including dynemicin A; bisphosphonates, such as clodronate; an esperamicin; as well as neocarzinostatin chromophore and related chromoprotein enediyne antibiotic chromophores), aclacinomysins, actinomycin, authramycin, azaserine, bleomycins, cactinomycin, carabicin, caminomycin, carzinophilin, chromomycinis, dactinomycin, daunorubicin, detorubicin, 6-diazo-5-oxo-L-norleucine, ADRIAMYCIN® (doxorubicin), morpholino-doxorubicin, cyanomorpholino-doxorubicin, 2-pyrrolino-doxorubicin and deoxydoxorubicin), epirubicin, esorubicin, idarubicin, marcellomycin, mitomycins such as mitomycin C, mycophenolic acid, nogalamycin, olivomycins, peplomycin, porfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogs such as denopterin, methotrexate, pteropterin, trimetrexate; purine analogs such as fludarabine, 6-mercaptopurine, thiamiprine, thioguanine; pyrimidine analogs such as ancitabine, azacitidine, 6 azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; eniluracil; amsacrine; bestrabucil; bisantrene; edatraxate; defofamine; demecolcine; diaziquone; elfomithine; elliptinium acetate; an epothilone; etoglucid; gallium nitrate; hydroxyurea; lentinan; lonidainine; maytansinoids such as maytansine and ansamitocins; mitoguazone; mitoxantrone; mopidamnol; nitraerine; pentostatin; phenamet; pirarubicin; losoxantrone; podophyllinic acid; 2-ethylhydrazide; procarbazine; PSK® polysaccharide complex (JHS Natural Products, Eugene, Oreg.); razoxane; rhizoxin; sizofuran; spirogermanium; tenuazonic acid; triaziquone; 2,2′,2″-trichlorotriethylamine; trichothecenes (especially T-2 toxin, verracurin A, roridin A and anguidine); urethan; vindesine; dacarbazine; mannomustine; mitobronitol; mitolactol; pipobroman; gacytosine; arabinoside (“Ara-C”); cyclophosphamide; thiotepa; taxoids, e.g., TAXOL (paclitaxel; Bristol-Myers Squibb Oncology, Princeton, N.J.), ABRAXANE® (Cremophor-free), albumin-engineered nanoparticle formulations of paclitaxel (American Pharmaceutical Partners, Schaumberg, Ill.), and TAXOTERE® (docetaxel, doxetaxel; Sanofi-Aventis); chloranmbucil; GEMZAR® (gemcitabine); 6-thioguanine; mercaptopurine; methotrexate; platinum analogs such as cisplatin and carboplatin; vinblastine; etoposide (VP-16); ifosfamide; mitoxantrone; vincristine; NAVELBINE® (vinorelbine); novantrone; teniposide; edatrexate; daunomycin; aminopterin; capecitabine (XELODA®); ibandronate; CPT-11; topoisomerase inhibitor RFS 2000; difluoromethylornithine (DMFO); retinoids such as retinoic acid; and pharmaceutically acceptable salts, acids and derivatives of any of the above.

Chemotherapeutic agent also includes (i) anti-hormonal agents that act to regulate or inhibit hormone action on tumors such as anti-estrogens and selective estrogen receptor modulators (SERMs), including, for example, tamoxifen (including NOLVADEX®; tamoxifen citrate), raloxifene, droloxifene, iodoxyfene, 4-hydroxytamoxifen, trioxifene, keoxifene, LY117018, onapristone, and FARESTON® (toremifine citrate); (ii) aromatase inhibitors that inhibit the enzyme aromatase, which regulates estrogen production in the adrenal glands, such as, for example, 4(5)-imidazoles, aminoglutethimide, MEGASE® (megestrol acetate), AROMASIN® (exemestane; Pfizer), formestanie, fadrozole, RIVISOR® (vorozole), FEMARA® (letrozole; Novartis), and ARIMIDEX® (anastrozole; AstraZeneca); (iii) anti-androgens such as flutamide, nilutamide, bicalutamide, leuprolide and goserelin; buserelin, tripterelin, medroxyprogesterone acetate, diethylstilbestrol, premarin, fluoxymesterone, all transretionic acid, fenretinide, as well as troxacitabine (a 1,3-dioxolane nucleoside cytosine analog); (iv) protein kinase inhibitors; (v) lipid kinase inhibitors; (vi) antisense oligonucleotides, particularly those which inhibit expression of genes in signaling pathways implicated in aberrant cell proliferation, such as, for example, PKC-alpha, Ralf and H-Ras; (vii) ribozymes such as VEGF expression inhibitors (e.g., ANGIOZYME®) and HER2 expression inhibitors; (viii) vaccines such as gene therapy vaccines, for example, ALLOVECTIN®, LEUVECTIN®, and VAXID®; PROLEUKIN®, rIL-2; a topoisomerase 1 inhibitor such as LURTOTECAN®; ABARELIX® rmRH; and (ix) pharmaceutically acceptable salts, acids and derivatives of any of the above.

Chemotherapeutic agent also includes antibodies such as alemtuzumab (Campath), bevacizumab (AVASTIN®, Genentech); cetuximab (ERBITUX®, Imclone); panitumumab (VECTIBIX®, Amgen), rituximab (RITUXAN®, Genentech/Biogen Idec), pertuzumab (OMNITARG®, 2C4, Genentech), trastuzumab (HERCEPTIN®, Genentech), tositumomab (Bexxar, Corixia), and the antibody drug conjugate, gemtuzumab ozogamicin (MYLOTARG®, Wyeth). Additional humanized monoclonal antibodies with therapeutic potential as agents in combination with the compounds of the invention include: apolizumab, aselizumab, atlizumab, bapineuzumab, bivatuzumab mertansine, cantuzumab mertansine, cedelizumab, certolizumab pegol, cidfusituzumab, cidtuzumab, daclizumab, eculizumab, efalizumab, epratuzumab, erlizumab, felvizumab, fontolizumab, gemtuzumab ozogamicin, inotuzumab ozogamicin, ipilimumab, labetuzumab, lintuzumab, matuzumab, mepolizumab, motavizumab, motovizumab, natalizumab, nimotuzumab, nolovizumab, numavizumab, ocrelizumab, omalizumab, palivizumab, pascolizumab, pecfusituzumab, pectuzumab, pexelizumab, ralivizumab, ranibizumab, reslivizumab, reslizumab, resyvizumab, rovelizumab, ruplizumab, sibrotuzumab, siplizumab, sontuzumab, tacatuzumab tetraxetan, tadocizumab, talizumab, tefibazumab, tocilizumab, toralizumab, tucotuzumab celmoleukin, tucusituzumab, umavizumab, urtoxazumab, ustekinumab, visilizumab, and the anti-interleukin-12 (ABT-874/J695, Wyeth Research and Abbott Laboratories) which is a recombinant exclusively human-sequence, full-length IgG1 λ antibody genetically modified to recognize interleukin-12 p40 protein.

Chemotherapeutic agent also includes “EGFR inhibitors,” which refers to compounds that bind to or otherwise interact directly with EGFR or its mutant forms and prevent or reduce its signaling activity, and is alternatively referred to as an “EGFR antagonist.” Examples of such agents include antibodies and small molecules that bind to EGFR. Examples of antibodies which bind to EGFR include MAb 579 (ATCC CRL HB 8506), MAb 455 (ATCC CRL HB8507), MAb 225 (ATCC CRL 8508), MAb 528 (ATCC CRL 8509) (see, U.S. Pat. No. 4,943,533, Mendelsohn et al.) and variants thereof, such as chimerized 225 (C225 or Cetuximab; ERBUTIX®) and reshaped human 225 (H225) (see, WO 96/40210, Imelone Systems Inc.); IMC-11F8, a fully human, EGFR-targeted antibody (Imclone); antibodies that bind type II mutant EGFR (U.S. Pat. No. 5,212,290); humanized and chimeric antibodies that bind EGFR as described in U.S. Pat. No. 5,891,996; and human antibodies that bind EGFR, such as ABX-EGF or Panitumumab (see WO98/50433, Abgenix/Amgen); EMD 55900 (Stragliotto et al. Eur. J. Cancer 32A:636-640 (1996)); EMD7200 (matuzumab) a humanized EGFR antibody directed against EGFR that competes with both EGF and TGF-alpha for EGFR binding (EMD/Merck); human EGFR antibody, HuMax-EGFR (GenMab); fully human antibodies known as E1.1, E2.4, E2.5, E6.2, E6.4, E2.11, E6. 3 and E7.6. 3 and described in U.S. Pat. No. 6,235,883; MDX-447 (Medarex Inc); and mAb 806 or humanized mAb 806 (Johns et al., J. Biol. Chem. 279(29):30375-30384 (2004)). The anti-EGFR antibody may be conjugated with a cytotoxic agent, thus generating an immunoconjugate (see, e.g., EP659,439A2, Merck Patent GmbH). EGFR antagonists include small molecules such as compounds described in U.S. Pat. Nos. 5,616,582, 5,457,105, 5,475,001, 5,654,307, 5,679,683, 6,084,095, 6,265,410, 6,455,534, 6,521,620, 6,596,726, 6,713,484, 5,770,599, 6,140,332, 5,866,572, 6,399,602, 6,344,459, 6,602,863, 6,391,874, 6,344,455, 5,760,041, 6,002,008, and 5,747,498, as well as the following PCT publications: WO98/14451, WO98/50038, WO99/09016, and WO99/24037. Particular small molecule EGFR antagonists include OSI-774 (CP-358774, erlotinib, TARCEVA® Genentech/OSI Pharmaceuticals); PD 183805 (CI 1033, 2-propenamide, N-[4-[(3-chloro-4-fluorophenyl)amino]-7-[3-(4-morpholinyl)propoxy]-6-quinazolinyl]-, dihydrochloride, Pfizer Inc.); ZD1839, gefitinib (IRESSA®) 4-(3′-Chloro-4′-fluoroanilino)-7-methoxy-6-(3-morpholinopropoxy)quinazoline, AstraZeneca); ZM 105180 ((6-amino-4-(3-methylphenyl-amino)-quinazoline, Zeneca); BIBX-1382 (N8-(3-chloro-4-fluoro-phenyl)-N2-(1-methyl-piperidin-4-yl)-pyrimido[5,4-d]pyrimidine-2,8-diamine, Boehringer Ingelheim); PKI-166 ((R)-4-[4-[(1-phenylethyl)amino]-1H-pyrrolo[2,3-d]pyrimidin-6-yl]-phenol); (R)-6-(4-hydroxyphenyl)-4-[(1-phenylethyl)amino]-7H-pyrrolo[2,3-d]pyrimidine); CL-387785 (N-[4-[(3-bromophenyl)amino]-6-quinazolinyl]-2-butynamide); EKB-569 (N-[4-[(3-chloro-4-fluorophenyl)amino]-3-cyano-7-ethoxy-6-quinolinyl]-4-(dimethylamino)-2-butenamide) (Wyeth); AG1478 (Pfizer); AG1571 (SU 5271; Pfizer); dual EGFR/IER2 tyrosine kinase inhibitors such as lapatinib (TYKERB®, GSK572016 or N-[3-chloro-4-[(3 fluorophenyl)methoxy]phenyl]-6[5[[[2methylsulfonyl)ethyl]amino]methyl]-2-furanyl]-4-quinazolinamine). Each of the above-described references is incorporated herein by reference in its entirety for all of its teachings, including without limitation all methods, compounds, compositions, data and the like, for use with any of the embodiments and disclosure herein.

Chemotherapeutic agents also include “tyrosine kinase inhibitors” including the EGFR-targeted drugs noted in the preceding paragraph; small molecule HER2 tyrosine kinase inhibitor such as TAK165 available from Takeda; CP-724,714, an oral selective inhibitor of the ErbB2 receptor tyrosine kinase (Pfizer and OSI); dual-HER inhibitors such as EKB-569 (available from Wyeth) which preferentially binds EGFR but inhibits both HER2 and EGFR-overexpressing cells; lapatinib (GSK572016; available from Glaxo-SmithKline), an oral HER2 and EGFR tyrosine kinase inhibitor; PKI-166 (available from Novartis); pan-HER inhibitors such as canertinib (CI-1033; Pharmacia); Raf-1 inhibitors such as antisense agent ISIS-5 132 available from ISIS Pharmaceuticals which inhibit Raf-1 signaling; non-HER targeted TK inhibitors such as imatinib mesylate (GLEEVEC®, available from Glaxo SmithKline); multi-targeted tyrosine kinase inhibitors such as sunitinib (SUTENT®, available from Pfizer); VEGF receptor tyrosine kinase inhibitors such as vatalanib (PTK787/ZK222584, available from Novartis/Schering AG); MAPK extracellular regulated kinase Iinhibitor CI-1040 (available from Pharmacia); quinazolines, such as PD 153035,4-(3-chloroanilino) quinazoline; pyridopyrimidines; pyrimidopyrimidines; pyrrolopyrimidines, such as CGP 59326, CGP 60261 and CGP 62706; pyrazolopyrimidines, 4-(phenylamino)-7H-pyrrolo[2,3-d] pyrimidines; curcumin (diferuloyl methane, 4,5-bis (4-fluoroanilino)phthalimide); tyrphostines containing nitrothiophene moieties; PD-0183805 (Warner-Lamber); antisense molecules (e.g. those that bind to HER-encoding nucleic acid); quinoxalines (U.S. Pat. No. 5,804,396); tryphostins (U.S. Pat. No. 5,804,396); ZD6474 (Astra Zeneca); PTK-787 (Novartis/Schering AG); pan-HER inhibitors such as CI-1033 (Pfizer); Affinitac (ISIS 3521; Isis/Lilly); imatinib mesylate (GLEEVEC®); PKI 166 (Novartis); GW2016 (Glaxo SmithKline); CI-1033 (Pfizer); EKB-569 (Wyeth); Semaxinib (Pfizer); ZD6474 (AstraZeneca); PTK-787 (Novartis/Schering AG); INC-1C11 (Imclone), rapamycin (sirolimus, RAPAMUNE®); or as described in any of the following patent publications: U.S. Pat. No. 5,804,396; WO 1999/09016 (American Cyanamid); WO 1998/43960 (American Cyanamid); WO 1997/38983 (Warner Lambert); WO 1999/06378 (Warner Lambert); WO 1999/06396 (Warner Lambert); WO 1996/30347 (Pfizer, Inc); WO 1996/33978 (Zeneca); WO 1996/3397 (Zeneca) and WO 1996/33980 (Zeneca). Each of the above-described references is incorporated herein by reference in its entirety for all of its teachings, including without limitation all methods, compounds, compositions, data and the like, for use with any of the embodiments and disclosure herein.

Chemotherapeutic agents also include dexamethasone, interferons, colchicine, metoprine, cyclosporine, amphotericin, metronidazole, alemtuzumab, alitretinoin, allopurinol, amifostine, arsenic trioxide, asparaginase, BCG live, bevacuzimab, bexarotene, cladribine, clofarabine, darbepoetin alfa, denileukin, dexrazoxane, epoetin alfa, elotinib, filgrastim, histrelin acetate, ibritumomab, interferon alfa-2a, interferon alfa-2b, lenalidomide, levamisole, mesna, methoxsalen, nandrolone, nelarabine, nofetumomab, oprelvekin, palifermin, pamidronate, pegademase, pegaspargase, pegfilgrastim, pemetrexed disodium, plicamycin, porfimer sodium, quinacrine, rasburicase, sargramostim, temozolomide, VM-26, 6-TG, toremifene, tretinoin, ATRA, valrubicin, zoledronate, and zoledronic acid, and pharmaceutically acceptable salts thereof.

Chemotherapeutic agents also include hydrocortisone, hydrocortisone acetate, cortisone acetate, tixocortol pivalate, triamcinolone acetonide, triamcinolone alcohol, mometasone, amcinonide, budesonide, desonide, fluocinonide, fluocinolone acetonide, betamethasone, betamethasone sodium phosphate, dexamethasone, dexamethasone sodium phosphate, fluocortolone, hydrocortisone-17-butyrate, hydrocortisone-17-valerate, aclometasone dipropionate, betamethasone valerate, betamethasone dipropionate, prednicarbate, clobetasone-17-butyrate, clobetasol-17-propionate, fluocortolone caproate, fluocortolone pivalate and fluprednidene acetate; immune selective anti-inflammatory peptides (ImSAIDs) such as phenylalanine-glutamine-glycine (FEG) and its D-isomeric form (feG) (IMULAN BioTherapeutics, LLC); anti-rheumatic drugs such as azathioprine, ciclosporin (cyclosporine A), D-penicillamine, gold salts, hydroxychloroquine, leflunomideminocycline, sulfasalazine, tumor necrosis factor alpha (TNFα) blockers such as etanercept (Enbrel), infliximab (Remicade), adalimumab (Humira), certolizumab pegol (Cimzia), golimumab (Simponi), Interleukin 1 (IL-1) blockers such as anakinra (Kineret), T cell costimulation blockers such as abatacept (Orencia), Interleukin 6 (IL-6) blockers such as tocilizumab (ACTEMERA®); Interleukin 13 (IL-13) blockers such as lebrikizumab; Interferon alpha (IFN) blockers such as Rontalizumab; Beta 7 integrin blockers such as rhuMAb Beta7; IgE pathway blockers such as Anti-M1 prime; Secreted homotrimeric LTa3 and membrane bound heterotrimer LTa1/β2 blockers such as Anti-lymphotoxin alpha (LTa); radioactive isotopes (e.g., At²¹¹, I¹³¹, I¹²⁵, Y⁹⁰, Re¹⁸⁶, Re¹⁸⁸, Sm¹⁵³, Bi²¹², P³², Pb²¹² and radioactive isotopes of Lu); miscellaneous investigational agents such as thioplatin, PS-341, phenylbutyrate, ET-18-OCH₃, or farnesyl transferase inhibitors (L-739749, L-744832); polyphenols such as quercetin, resveratrol, piceatannol, epigallocatechine gallate, theaflavins, flavanols, procyanidins, betulinic acid and derivatives thereof, autophagy inhibitors such as chloroquine; delta-9-tetrahydrocannabinol (dronabinol, MARINOL®); beta-lapachone; lapachol; colchicines; betulinic acid; acetylcamptothecin, scopolectin, and 9-aminocamptothecin); podophyllotoxin; tegafur (UFTORAL®); bexarotene (TARGRETIN®); bisphosphonates such as clodronate (for example, BONEFOS® or OSTAC®), etidronate (DIDROCAL®), NE-58095, zoledronic acid/zoledronate (ZOMETA®), alendronate (FOSAMAX®), pamidronate (AREDIA®), tiludronate (SKELID®), or risedronate (ACTONEL®); and epidermal growth factor receptor (EGF-R); vaccines such as THERATOPE® vaccine; perifosine, COX-2 inhibitor (e.g. celecoxib or etoricoxib), proteosome inhibitor (e.g. PS341); CCI-779; tipifarnib (R11577); orafenib, ABT510; Bcl-2 inhibitor such as oblimersen sodium (GENASENSE®); pixantrone; farnesyltransferase inhibitors such as lonafarnib (SCH 6636, SARASAR™); and pharmaceutically acceptable salts, acids or derivatives of any of the above; as well as combinations of two or more of the above such as CHOP, an abbreviation for a combined therapy of cyclophosphamide, doxorubicin, vincristine, and prednisolone; and FOLFOX, an abbreviation for a treatment regimen with oxaliplatin (ELOXATIN™) combined with 5-FU and leucovorin.

Chemotherapeutic agents also include non-steroidal anti-inflammatory drugs with analgesic, antipyretic and anti-inflammatory effects. NSAIDs include non-selective inhibitors of the enzyme cyclooxygenase. Specific examples of NSAIDs include aspirin, propionic acid derivatives such as ibuprofen, fenoprofen, ketoprofen, flurbiprofen, oxaprozin and naproxen, acetic acid derivatives such as indomethacin, sulindac, etodolac, diclofenac, enolic acid derivatives such as piroxicam, meloxicam, tenoxicam, droxicam, lornoxicam and isoxicam, fenamic acid derivatives such as mefenamic acid, meclofenamic acid, flufenamic acid, tolfenamic acid, and COX-2 inhibitors such as celecoxib, etoricoxib, lumiracoxib, parecoxib, rofecoxib, and valdecoxib. NSAIDs can be indicated for the symptomatic relief of conditions such as rheumatoid arthritis, osteoarthritis, inflammatory arthropathies, ankylosing spondylitis, psoriatic arthritis, Reiter's syndrome, acute gout, dysmenorrhoea, metastatic bone pain, headache and migraine, postoperative pain, mild-to-moderate pain due to inflammation and tissue injury, pyrexia, ileus, and renal colic.

In certain embodiments, chemotherapeutic agents include, but are not limited to, doxorubicin, dexamethasone, vincristine, cyclophosphamide, fluorouracil, topotecan, interferons, platinum derivatives, taxanes (e.g., paclitaxel, docetaxel), vinca alkaloids (e.g., vinblastine), anthracyclines (e.g., doxorubicin), epipodophyllotoxins (e.g., etoposide), cisplatin, an mTOR inhibitor (e.g., a rapamycin), methotrexate, actinomycin D, dolastatin 10, colchicine, trimetrexate, metoprine, cyclosporine, daunorubicin, teniposide, amphotericin, alkylating agents (e.g., chlorambucil), 5-fluorouracil, campthothecin, cisplatin, metronidazole, and imatinib mesylate, among others. In other embodiments, a compound disclosed herein is administered in combination with a biologic agent, such as bevacizumab or panitumumab.

In certain embodiments, compounds disclosed herein, or a pharmaceutically acceptable composition thereof, are administered in combination with an antiproliferative or chemotherapeutic agent selected from any one or more of abarelix, aldesleukin, alemtuzumab, alitretinoin, allopurinol, altretamine, amifostine, anastrozole, arsenic trioxide, asparaginase, azacitidine, BCG live, bevacuzimab, fluorouracil, bexarotene, bleomycin, bortezomib, busulfan, calusterone, capecitabine, camptothecin, carboplatin, carmustine, cetuximab, chlorambucil, cladribine, clofarabine, cyclophosphamide, cytarabine, dactinomycin, darbepoetin alfa, daunorubicin, denileukin, dexrazoxane, docetaxel, doxorubicin (neutral), doxorubicin hydrochloride, dromostanolone propionate, epirubicin, epoetin alfa, elotinib, estramustine, etoposide phosphate, etoposide, exemestane, filgrastim, floxuridine, fludarabine, fulvestrant, gefitinib, gemcitabine, gemtuzumab, goserelin acetate, histrelin acetate, hydroxyurea, ibritumomab, idarubicin, ifosfamide, imatinib mesylate, interferon alfa-2a, interferon alfa-2b, irinotecan, lenalidomide, letrozole, leucovorin, leuprolide acetate, levamisole, lomustine, megestrol acetate, melphalan, mercaptopurine, 6-MP, mesna, methotrexate, methoxsalen, mitomycin C, mitotane, mitoxantrone, nandrolone, nelarabine, nofetumomab, oprelvekin, oxaliplatin, paclitaxel, palifermin, pamidronate, pegademase, pegaspargase, pegfilgrastim, pemetrexed disodium, pentostatin, pipobroman, plicamycin, porfimer sodium, procarbazine, quinacrine, rasburicase, rituximab, sargramostim, sorafenib, streptozocin, sunitinib maleate, talc, tamoxifen, temozolomide, teniposide, VM-26, testolactone, thioguanine, 6-TG, thiotepa, topotecan, toremifene, tositumomab, trastuzumab, tretinoin, ATRA, uracil mustard, valrubicin, vinblastine, vincristine, vinorelbine, zoledronate, or zoledronic acid.

In some embodiments, the dosing of the compound of Formula I can be in any suitable amount to treat the cancer. For example, the dosing could be a daily dosage of between 1 mg weight up to 500 mg. As an additional example, the daily dose could be in a range from about 20 mg to 400 mg (or any sub-range or sub-value there between, including endpoints). In some embodiments, the range of dosing of the compound of Formula I can be from 10 mg to 300 mg. In some embodiments, the range of dosing of the compound of Formula I can be from 10 mg to 100 mg. In some embodiments, the range of dosing of the compound of Formula I can be from 5 mg to 50 mg. The daily dosage can be achieved by administering a single administered dosage (e.g., QD) or via multiple administrations during a day (e.g., BID, TID, QID, etc.) to provide the total daily dosage. In some embodiments, the dosing of the MEK inhibitor is any suitable amount. For example, it can be an amount in a range from 1 mg to 500 mg daily (or any sub-range or sub-value there between, including endpoints). Dosing of the MEK inhibitor may be the same or less than the approved dosing for any given MEK inhibitor and may depend on a given indication. In some embodiments, trametinib may be administered at a dose in a range from about 1 mg to about 10 mg, once daily. For example, trametinib is approved for 2 mg once daily. It is also approved at dose reductions such as 1.5 mg QD and 1 mg QD. In some embodiments, binimetinib may be administered at a dose in a range from about 30 mg to about 100 mg. For example, binimetinib is approved for 45 mg doses, twice daily. Binimetinib is also approved at dose reductions, such as about 30 mg BID. It will be appreciated that each of the recited ranges above can include any sub-range or sub-point therein, inclusive of endpoints. It will be appreciated that each of the recited ranges above can include any sub-range or sub-point therein, inclusive of endpoints. A common dose range for adult humans is generally from 5 mg to 2 g/day. Tablets or other forms of presentation provided in discrete units may conveniently contain an amount of one or more compounds which is effective at such dosage or as a multiple of the same, for instance, units containing 5 mg to 500 mg, usually around 10 mg to 200 mg. The amount of active ingredient that may be combined with the carrier materials to produce a single dosage form will vary depending upon the host treated and the particular mode of administration. In some embodiments, the administration is oral.

In some embodiments, there are provided methods of treating colorectal and NSCLC cancer in a subject comprising orally administering to the subject a therapeutically effective amount of a compound of Formula I or its pharmaceutically acceptable salt in combination with trametinib or binimetinib. In some embodiments, the compound of Formula I is administered once or twice daily. In some embodiments, trametinib or binimetinib may be administered once or twice daily. The drugs can be co-administered as described herein, for example.

In some embodiments, the subject is a human. In some embodiments, the subject is a mammal other than a human, such as a primate, a rodent a dog, a cat, or other small animal.

In some embodiments, there are provided methods of inhibiting ERK1/2 phosphorylation comprising contacting a cell population with Formula I or its pharmaceutically acceptable salt in combination with trametinib or binimetinib. In some embodiments, a concentration of the compound of Formula I is in a range from 1 nm to 1 micromolar, or from 1 nm, to 500 nM, or 1 nM to 20 nM. In some embodiments, a concentration of trametinib or binimetinib is in a range from 10 nM to 1 micromolar, or from 10 nM to 500 nM.

### Compositions

The compound of Formula I disclosed herein may exist as salts. The present embodiments include such salts, which can be pharmaceutically acceptable salts. Examples of applicable salt forms include hydrochlorides, hydrobromides, sulfates, methanesulfonates, nitrates, maleates, acetates, citrates, fumarates, tartrates (eg (+)-tartrates, (−)-tartrates or mixtures thereof including racemic mixtures, succinates, benzoates and salts with amino acids such as glutamic acid. These salts may be prepared by methods known to those skilled in art. Also included are base addition salts such as sodium, potassium, calcium, ammonium, organic amino, or magnesium salt, or a similar salt. When compounds of the present embodiments contain relatively basic functionalities, acid addition salts can be obtained by contacting the neutral form of such compounds with a sufficient amount of the desired acid, either neat or in a suitable inert solvent. Examples of acceptable acid addition salts include those derived from inorganic acids like hydrochloric, hydrobromic, nitric, carbonic, monohydrogencarbonic, phosphoric, monohydrogenphosphoric, dihydrogenphosphoric, sulfuric, monohydrogensulfuric, hydriodic, or phosphorous acids and the like, as well as the salts derived organic acids like acetic, propionic, isobutyric, maleic, malonic, benzoic, succinic, suberic, fumaric, lactic, mandelic, phthalic, benzenesulfonic, p-tolylsulfonic, citric, tartaric, methanesulfonic, and the like. Also included are salts of amino acids such as arginate and the like, and salts of organic acids like glucuronic or galactunoric acids and the like. Certain specific compounds of the present embodiments contain both basic and acidic functionalities that allow the compounds to be converted into either base or acid addition salts.

Other salts include acid or base salts of the compounds used in the methods of the present embodiments. Illustrative examples of pharmaceutically acceptable salts are mineral acid (hydrochloric acid, hydrobromic acid, phosphoric acid, and the like) salts, organic acid (acetic acid, propionic acid, glutamic acid, citric acid and the like) salts, and quaternary ammonium (methyl iodide, ethyl iodide, and the like) salts. It is understood that the pharmaceutically acceptable salts are non-toxic. Additional information on suitable pharmaceutically acceptable salts can be found in Remington's Pharmaceutical Sciences, 17th ed., Mack Publishing Company, Easton, Pa., 1985, which is incorporated herein by reference in its entirety for all of its teachings, including without limitation all methods, compounds, compositions, data and the like, for use with any of the embodiments and disclosure herein.

Pharmaceutically acceptable salts include salts of the active compounds which are prepared with relatively nontoxic acids or bases, depending on the particular substituents found on the compounds described herein. When compounds of the present embodiments contain relatively acidic functionalities, base addition salts can be obtained by contacting the neutral form of such compounds with a sufficient amount of the desired base, either neat or in a suitable inert solvent. Examples of pharmaceutically acceptable base addition salts include sodium, potassium, calcium, ammonium, organic amino, or magnesium salt, or a similar salt. When compounds of the present embodiments contain relatively basic functionalities, acid addition salts can be obtained by contacting the neutral form of such compounds with a sufficient amount of the desired acid, either neat or in a suitable inert solvent. Examples of pharmaceutically acceptable acid addition salts include those derived from inorganic acids like hydrochloric, hydrobromic, nitric, carbonic, monohydrogencarbonic, phosphoric, monohydrogenphosphoric, dihydrogenphosphoric, sulfuric, monohydrogensulfuric, hydriodic, or phosphorous acids and the like, as well as the salts derived from relatively nontoxic organic acids like acetic, propionic, isobutyric, maleic, malonic, benzoic, succinic, suberic, fumaric, lactic, mandelic, phthalic, benzenesulfonic, p-tolylsulfonic, citric, tartaric, methanesulfonic, and the like. Also included are salts of amino acids such as arginate and the like, and salts of organic acids like glucuronic or galactunoric acids and the like (see, for example, Berge et al., “Pharmaceutical Salts”, Journal of Pharmaceutical Science, 1977, 66, 1-19), which is incorporated herein by reference in its entirety for all of its teachings, including without limitation all methods, compounds, compositions, data and the like, for use with any of the embodiments and disclosure herein. Certain specific compounds of the present embodiments contain both basic and acidic functionalities that allow the compounds to be converted into either base or acid addition salts.

The neutral forms of the compounds are preferably regenerated by contacting the salt with abase or acid and isolating the parent compound in the conventional manner. The parent form of the compound differs from the various salt forms in certain physical properties, such as solubility in polar solvents.

Certain compounds of the present embodiments can exist in unsolvated forms as well as solvated forms, including hydrated forms. In general, the solvated forms are equivalent to unsolvated forms and are encompassed within the scope of the present embodiments. Certain compounds of the present embodiments may exist in multiple crystalline or amorphous forms. In general, all physical forms are equivalent for the uses contemplated by the present embodiments and are intended to be within the scope of the present embodiments.

Certain compounds of the present embodiments possess asymmetric carbon atoms (optical centers) or double bonds; the enantiomers, racemates, diastereomers, tautomers, geometric isomers, stereoisomeric forms that may be defined, in terms of absolute stereochemistry, as (R)- or (S)- or, as (D)- or (L)- for amino acids, and individual isomers are encompassed within the scope of the present embodiments. The compounds of the present embodiments do not include those which are known in art to be too unstable to synthesize and/or isolate. The present embodiments is meant to include compounds in racemic and optically pure forms. Optically active (R)- and (S)-, or (D)- and (L)-isomers may be prepared using chiral synthons or chiral reagents or resolved using conventional techniques.

Unless otherwise stated, the compounds of the present embodiments may also contain unnatural proportions of atomic isotopes at one or more of the atoms that constitute such compounds. For example, the compounds of the present embodiments may be labeled with radioactive or stable isotopes, such as for example deuterium (²H), tritium (³H), iodine-125 (¹²⁵I), fluorine-18 (¹⁸F), nitrogen-15 (¹⁵N), oxygen-17 (¹⁷O), oxygen-18 (¹⁸O), carbon-13 (¹³C), or carbon-14 (¹⁴C). All isotopic variations of the compounds of the present embodiments, whether radioactive or not, are encompassed within the scope of the present embodiments.

In addition to salt forms, the present embodiments provide compounds, which are in a prodrug form. Prodrugs of the compounds described herein are those compounds that readily undergo chemical changes under physiological conditions to provide the compounds of the present embodiments. Additionally, prodrugs can be converted to the compounds of the present embodiments by chemical or biochemical methods in an ex vivo environment. For example, prodrugs can be slowly converted to the compounds of the present embodiments when placed in a transdermal patch reservoir with a suitable enzyme or chemical reagent.

In some embodiments, there are provided pharmaceutical compositions comprising the compound of Formula I and a pharmaceutically acceptable excipient. In some embodiments, the pharmaceutical compositions are configured as an oral tablet preparation.

The compounds of the present embodiments can be prepared and administered in a wide variety of oral, parenteral and topical dosage forms. Oral preparations include tablets, pills, powder, dragees, capsules, liquids, lozenges, gels, syrups, slurries, suspensions, etc., suitable for ingestion by the patient. The compounds of the present embodiments can also be administered by injection, that is, intravenously, intramuscularly, intracutaneously, subcutaneously, intraduodenally, or intraperitoneally. Also, the compounds described herein can be administered by inhalation, for example, intranasally. Additionally, the compounds of the present embodiments can be administered transdermally. The compounds of formula I disclosed herein can also be administered by in intraocular, intravaginal, and intrarectal routes including suppositories, insufflation, powders and aerosol formulations (for examples of steroid inhalants, see Rohatagi, J. Clin. Pharmacol. 35:1187-1193, 1995; Tjwa, Ann. Allergy Asthma Immunol. 75:107-111, 1995), which is incorporated herein by reference in its entirety for all of its teachings, including without limitation all methods, compounds, compositions, data and the like, for use with any of the embodiments and disclosure herein. Accordingly, the present embodiments also provides pharmaceutical compositions including one or more pharmaceutically acceptable carriers and/or excipients and either a compound of formula I, or a pharmaceutically acceptable salt of a compound of formula I.

For preparing pharmaceutical compositions from the compounds of the present embodiments, pharmaceutically acceptable carriers can be either solid or liquid. Solid form preparations include powders, tablets, pills, capsules, cachets, suppositories, and dispersible granules. A solid carrier can be one or more substances, which may also act as diluents, flavoring agents, surfactants, binders, preservatives, tablet disintegrating agents, or an encapsulating material. Details on techniques for formulation and administration are well described in the scientific and patent literature, see, e.g., the latest edition of Remington's Pharmaceutical Sciences, Maack Publishing Co, Easton PA (“Remington's”), which is incorporated herein by reference in its entirety for all of its teachings, including without limitation all methods, compounds, compositions, data and the like, for use with any of the embodiments and disclosure herein.

In powders, the carrier is a finely divided solid, which is in a mixture with the finely divided active component. In tablets, the active component is mixed with the carrier having the necessary binding properties and additional excipients as required in suitable proportions and compacted in the shape and size desired.

The powders, capsules and tablets preferably contain from 5% or 10% to 70% of the active compound. Suitable carriers are magnesium carbonate, magnesium stearate, talc, sugar, lactose, pectin, dextrin, starch, gelatin, tragacanth, methylcellulose, sodium carboxymethylcellulose, a low melting wax, cocoa butter, and the like. The term “preparation” is intended to include the formulation of the active compound with encapsulating material as a carrier providing a capsule in which the active component with or without other excipients, is surrounded by a carrier, which is thus in association with it. Similarly, cachets and lozenges are included. Tablets, powders, capsules, pills, cachets, and lozenges can be used as solid dosage forms suitable for oral administration.

Suitable solid excipients are carbohydrate or protein fillers including, but not limited to sugars, including lactose, sucrose, mannitol, or sorbitol; starch from corn, wheat, rice, potato, or other plants; cellulose such as methyl cellulose, hydroxypropylmethylcellulose, or sodium carboxymethylcellulose; and gums including arabic and tragacanth; as well as proteins such as gelatin and collagen. If desired, disintegrating or solubilizing agents may be added, such as the cross-linked polyvinyl pyrrolidone, agar, alginic acid, or a salt thereof, such as sodium alginate.

Dragee cores are provided with suitable coatings such as concentrated sugar solutions, which may also contain gum arabic, talc, polyvinylpyrrolidone, carbopol gel, polyethylene glycol, and/or titanium dioxide, lacquer solutions, and suitable organic solvents or solvent mixtures. Dyestuffs or pigments may be added to the tablets or dragee coatings for product identification or to characterize the quantity of active compound (i.e., dosage). Pharmaceutical preparations disclosed herein can also be used orally using, for example, push-fit capsules made of gelatin, as well as soft, sealed capsules made of gelatin and a coating such as glycerol or sorbitol. Push-fit capsules can contain the compounds of formula I mixed with a filler or binders such as lactose or starches, lubricants such as talc or magnesium stearate, and, optionally, stabilizers. In soft capsules, the compounds of formula I may be dissolved or suspended in suitable liquids, such as fatty oils, liquid paraffin, or liquid polyethylene glycol with or without stabilizers.

Liquid form preparations include solutions, suspensions, and emulsions, for example, water or water/propylene glycol solutions. For parenteral injection, liquid preparations can be formulated in solution in aqueous polyethylene glycol solution.

Aqueous solutions suitable for oral use can be prepared by dissolving the active component in water and adding suitable colorants, flavors, stabilizers, and thickening agents as desired. Aqueous suspensions suitable for oral use can be made by dispersing the finely divided active component in water with viscous material, such as natural or synthetic gums, resins, methylcellulose, sodium carboxymethylcellulose, hydroxypropylmethylcellulose, sodium alginate, polyvinylpyrrolidone, gum tragacanth and gum acacia, and dispersing or wetting agents such as a naturally occurring phosphatide (e.g., lecithin), a condensation product of an alkylene oxide with a fatty acid (e.g., polyoxyethylene stearate), a condensation product of ethylene oxide with a long chain aliphatic alcohol (e.g., heptadecaethylene oxycetanol), a condensation product of ethylene oxide with a partial ester derived from a fatty acid and a hexitol (e.g., polyoxyethylene sorbitol mono-oleate), or a condensation product of ethylene oxide with a partial ester derived from fatty acid and a hexitol anhydride (e.g., polyoxyethylene sorbitan mono-oleate). The aqueous suspension can also contain one or more preservatives such as ethyl or n-propyl p-hydroxybenzoate, one or more coloring agents, one or more flavoring agents and one or more sweetening agents, such as sucrose, aspartame or saccharin. Formulations can be adjusted for osmolarity.

Also included are solid form preparations, which are intended to be converted, shortly before use, to liquid form preparations for oral administration. Such liquid forms include solutions, suspensions, and emulsions. These preparations may contain, in addition to the active component, colorants, flavors, stabilizers, buffers, artificial and natural sweeteners, dispersants, thickeners, solubilizing agents, and the like.

Oil suspensions can be formulated by suspending the compound of formula I in a vegetable oil, such as arachis oil, olive oil, sesame oil or coconut oil, or in a mineral oil such as liquid paraffin; or a mixture of these. The oil suspensions can contain a thickening agent, such as beeswax, hard paraffin or cetyl alcohol. Sweetening agents can be added to provide a palatable oral preparation, such as glycerol, sorbitol or sucrose. These formulations can be preserved by the addition of an antioxidant such as ascorbic acid. As an example of an injectable oil vehicle, see Minto, J. Pharmacol. Exp. Ther. 281:93-102, 1997, which is incorporated herein by reference in its entirety for all of its teachings, including without limitation all methods, compounds, compositions, data and the like, for use with any of the embodiments and disclosure herein. The pharmaceutical formulations disclosed herein can also be in the form of oil-in-water emulsions. The oily phase can be a vegetable oil or a mineral oil, described above, or a mixture of these. Suitable emulsifying agents include naturally-occurring gums, such as gum acacia and gum tragacanth, naturally occurring phosphatides, such as soybean lecithin, esters or partial esters derived from fatty acids and hexitol anhydrides, such as sorbitan mono-oleate, and condensation products of these partial esters with ethylene oxide, such as polyoxyethylene sorbitan mono-oleate. The emulsion can also contain sweetening agents and flavoring agents, as in the formulation of syrups and elixirs. Such formulations can also contain a demulcent, a preservative, or a coloring agent.

The pharmaceutical formulations of the compound of Formula I disclosed herein can be provided as a salt and can be formed with bases, namely cationic salts such as alkali and alkaline earth metal salts, such as sodium, lithium, potassium, calcium, magnesium, as well as ammonium salts, such as ammonium, trimethyl-ammonium, diethylammonium, and tris-(hydroxymethyl)-methyl-ammonium salts.

The pharmaceutical preparation is preferably in unit dosage form. In such form the preparation is subdivided into unit doses containing appropriate quantities of the active component. The unit dosage form can be a packaged preparation, the package containing discrete quantities of preparation, such as packeted tablets, capsules, and powders in vials or ampoules. Also, the unit dosage form can be a capsule, tablet, cachet, or lozenge itself, or it can be the appropriate number of any of these in packaged form.

The quantity of active component in a unit dose preparation may be varied or adjusted from 0.1 mg to 10000 mg, more typically 1.0 mg to 1000 mg, most typically 10 mg to 500 mg, according to the particular application and the potency of the active component. The composition can, if desired, also contain other compatible therapeutic agents.

The dosage regimen also takes into consideration pharmacokinetics parameters well known in the art, i.e., the rate of absorption, bioavailability, metabolism, clearance, and the like (see, e.g., Hidalgo-Aragones (1996) J. Steroid Biochem. Mol. Biol. 58:611-617; Groning (1996) Pharmazie 51:337-341; Fotherby (1996) Contraception 54:59-69; Johnson (1995) J. Pharm. Sci. 84:1144-1146; Rohatagi (1995) Pharmazie 50:610-613; Brophy (1983) Eur. J. Clin. Pharmacol. 24:103-108; the latest Remington's, supra; each of which is incorporated herein by reference in its entirety for all of its teachings, including without limitation all methods, compounds, compositions, data and the like, for use with any of the embodiments and disclosure herein). The state of the art allows the clinician to determine the dosage regimen for each individual patient, GR and/or MR modulator and disease or condition treated.

Single or multiple administrations of the compound of Formula I formulations can be administered depending on the dosage and frequency as required and tolerated by the patient. The formulations should provide a sufficient quantity of active agent to effectively treat the disease state. Thus, in one embodiment, the pharmaceutical formulations for oral administration of the compound of formula I is in a daily amount of between about 0.5 to about 30 mg per kilogram of body weight per day, including all sub-ranges and sub-values therein, inclusive of endpoints. In an alternative embodiment, dosages are from about 1 mg to about 20 mg per kg of body weight per patient per day are used. Lower dosages can be used, particularly when the drug is administered to an anatomically secluded site, such as the cerebral spinal fluid (CSF) space, in contrast to administration orally, into the blood stream, into a body cavity or into a lumen of an organ. Substantially higher dosages can be used in topical administration. Actual methods for preparing formulations including the compound of formula I for parenteral administration are known or apparent to those skilled in the art and are described in more detail in such publications as Remington's, supra. See also Nieman, In “Receptor Mediated Antisteroid Action,” Agarwal, et al., eds., De Gruyter, New York (1987), which is incorporated herein by reference in its entirety for all of its teachings, including without limitation all methods, compounds, compositions, data and the like, for use with any of the embodiments and disclosure herein.

In some embodiments, co-administration includes administering one active agent within 0.5, 1, 2, 4, 6, 8, 10, 12, 16, 20, or 24 hours (or any sub-range of time or sub-value of time within a 24 hour period) of a second active agent. Co-administration includes administering two active agents simultaneously, approximately simultaneously (e.g., within about 1, 5, 10, 15, 20, or 30 minutes of each other (or any sub-range of time or sub-value of time from 0-30 minutes for example)), or sequentially in any order. In some embodiments, co-administration can be accomplished by co-formulation, i.e., preparing a single pharmaceutical composition including both active agents. In some embodiments, the active agents can be formulated separately. In some embodiments, the active and/or adjunctive agents may be linked or conjugated to one another. At least one administered dose of drugs can be administered, for example, at the same time. At least one administered dose of the drugs can be administered, for example, within minutes or less than an hour of each other. At least one administered dose of drugs can be administered, for example, at different times, but on the same day, or on different days.

After a pharmaceutical composition including a compound of formula I disclosed herein has been formulated in one or more acceptable carriers, it can be placed in an appropriate container and labeled for treatment of an indicated condition. For administration of the compounds of formula I, such labeling would include, e.g., instructions concerning the amount, frequency and method of administration.

### Pharmaceutical Dosing

The dosage regimen for the compounds herein will, of course, vary depending upon known factors, such as the pharmacodynamic characteristics of the particular agent and its mode and route of administration; the species, age, sex, health, medical condition, and weight of the recipient; the nature and extent of the symptoms; the kind of concurrent treatment; the frequency of treatment; the route of administration, the renal and hepatic function of the patient, and the effect desired. A clinical practitioner can determine and prescribe the effective amount of the drug required to prevent, counter, or arrest the progress of the disease or disorder.

By way of general guidance, the daily oral dosage of each active ingredient, when used for the indicated effects, will range between about 0.001 to about 1000 mg/kg of body weight, preferably between about 0.01 to about 100 mg/kg of body weight per day, an d most preferably between about 0.1 to about 20 mg/kg/day. In some embodiments, a compound of Formula (I) may be administered at a dose of between about 10 mg/day and about 200 mg/day. In some embodiments, a compound of Formula (I) may be administered at a dose of about 10 mg/day, 20 mg/day, 30 mg/day, 40 mg/day, 50 mg/day, 60 mg/day, 70 mg/day, 80 mg/day, 90 mg/day, 100 mg/day, 110 mg/day, 120 mg/day, 130 mg/day, 140 mg/day, 150 mg/day, 160 mg/day, 170 mg/day, 180 mg/day, 190 mg/day, or 200 mg/day. The dose may be any value or subrange within the recited ranges.

Depending on the patient's condition and the intended therapeutic effect, the dosing frequency for the therapeutic agent may vary, for example, from once per day to six times per day. That is, the dosing frequency may be QD, i.e., once per day, BID, i.e., twice per day; TID, i.e., three times per day; QID, i.e., four times per day; five times per day, or six times per day. In another embodiment, dosing frequency may be BIW, i.e., twice weekly, TIW, i.e., three times a week, or QIW, i.e. four times a week.

Depending on the patient's condition and the intended therapeutic effect, the treatment cycle may have a period of time where no therapeutic agent is administered. As used herein, “interval administration” refers to administration of the therapeutic agent followed by void days or void weeks. For example, the treatment cycle may be 3 weeks long which includes 2 weeks of dosing of the therapeutic agent(s) followed by 1 week where no therapeutic agent is administered. In some embodiments, the treatment cycle is 4 weeks long which includes 3 weeks of dosing followed by 1 week where no therapeutic agent is administered.

The term “treatment cycle” as used herein, means a pre-determined period of time for administering the therapeutic agent. Typically, the patient is examined at the end of each treatment cycle to evaluate the effect of the therapy.

In one embodiment, each of the treatment cycle has about 3 or more days. In another embodiment, each of the treatment cycle has from about 3 days to about 60 days. In another embodiment, each of the treatment cycle has from about 5 days to about 50 days. In another embodiment, each of the treatment cycle has from about 7 days to about 28 days. In another embodiment, each of the treatment cycle has 28 days. In one embodiment, the treatment cycle has about 29 days. In another embodiment, the treatment cycle has about 30 days. In another embodiment, the treatment cycle has about 31 days. In another embodiment, the treatment cycle has about a month-long treatment cycle. In another embodiment, the treatment cycle is any length of time from 3 weeks to 8 weeks. In another embodiment, the treatment cycle is any length of time from 3 weeks to 6 weeks. In yet another embodiment, the treatment cycle is 3 weeks. In another embodiment, the treatment cycle is one month. In another embodiment, the treatment cycle is 4 weeks. In another embodiment, the treatment cycle is 5 weeks. In another embodiment, the treatment cycle is 6 weeks. In another embodiment, the treatment cycle is 7 weeks. In another embodiment, the treatment cycle is 8 weeks. The duration of the treatment cycle may include any value or subrange within the recited ranges, including endpoints.

As used herein, the term “co-administration” or “coadministration” refers to administration of (a) an additional therapeutic agent and (b) a compound of Formula (I), or a salt, solvate, ester and/or prodrug thereof, together in a coordinated fashion. For example, the co-administration can be simultaneous administration, sequential administration, overlapping administration, interval administration, continuous administration, or a combination thereof.

In some embodiments, the dosing regimen for a compound of Formula (I) is once daily over a continuous 28-day cycle. In some embodiments, the once daily dosing regimen for a compound of Formula (I) may be, but is not limited to, 20 mg/day, 30 mg/day, 40 mg/day, 50 mg/day, 60 mg/day. Compounds of Formula (I) may be administered anywhere from 20 mg to 60 mg once a day. The dose may be any value or subrange within the recited ranges.

In some embodiments, the dosing regimen for a compound of Formula (I) is twice daily over a continuous 28-day cycle. In some embodiments, the twice daily dosing regimen for a compound of Formula (I) may be, but is not limited to, 10 mg/day, 20 mg/day, 30 mg/day, 40 mg/day, 50 mg/day, 60 mg/day, 70 mg/day, 80 mg/day, 90 mg/day, 100 mg/day. Compounds of Formula (I) may be administered anywhere from 20 mg to 80 mg twice a day. In some embodiments, compounds of Formula (I) may be administered anywhere from 10 mg/day to 100 mg/day. The dose may be any value or subrange within the recited ranges.

In some embodiments, the dosing regimen for a compound of Formula (I) may be once daily, anywhere from 20 mg to 60 mg per day for two weeks, followed by a one week break over a period of 6 weeks (e.g. 2 weeks on, 1 week off). In some embodiments, the dosing regimen for a compound of Formula (I) may be twice daily, anywhere from 10 mg to 100 mg twice a day for two weeks, followed by a one week break over a period of 6 weeks (e.g. 2 weeks on, 1 week off).

In some embodiments, the dosing regimen for a compound of Formula (I) may be once daily, anywhere from 20 mg to 60 mg per day for three weeks, followed by a one week break over a period of 8 weeks (e.g. 3 weeks on, 1 week off). In some embodiments, the dosing regimen for a compound of Formula (I) may be twice daily, anywhere from 10 mg to 100 mg twice a day for three weeks, followed by a one week break over a period of 8 weeks (e.g. 8 weeks on, 1 week off).

In some embodiments, the dosing regimen for a compound of Formula (I) may be twice daily on days 1 and 2, weekly for 8 weeks. In some embodiments, the dosing amount for compounds of Formula (I) may be, but is not limited to, 10 mg, 20 mg, 30 mg, 40 mg, 50 mg, 60 mg, 70 mg, 80 mg, 90 mg, 100 mg.

In some embodiments, the compound of Formula I, or a pharmaceutically acceptable salt thereof, is administered once a day for a 3-week cycle, comprising 2 weeks of administration of the compound followed by 1 week of no administration of the compound.

In some embodiments, the compound of Formula I, or a pharmaceutically acceptable salt thereof, is administered once a day for a 4-week cycle, comprising 3 weeks of administration of the compound followed by 1 week of no administration of the compound.

In some embodiments, the compound of Formula I, or a pharmaceutically acceptable salt thereof, is administered over a period of 6 weeks. In some embodiments, the compound of Formula I, or a pharmaceutically acceptable salt thereof, is administered over a period of 8 weeks.

In some embodiments, the compound of Formula I, or a pharmaceutically acceptable salt thereof, is administered 3 times a week. In some embodiments, the compound of Formula I, or a pharmaceutically acceptable salt thereof, is administered on day 1, day 3, and day 5 of the week.

In some embodiments, the compound of Formula I, or a pharmaceutically acceptable salt thereof, is administered 4 times a week.

In some embodiments, the compound of Formula I, or a pharmaceutically acceptable salt thereof, is administered for a 3-week cycle, comprising 2 weeks of administration of the compound followed by 1 week of no administration of the compound.

In some embodiments, the compound of Formula I, or a pharmaceutically acceptable salt thereof, is administered for a 4-week cycle, comprising 3 weeks of administration of the compound followed by 1 week of no administration of the compound.

In some embodiments, the compound of Formula I, or a pharmaceutically acceptable salt thereof, is administered twice a day, two days per week. In some embodiments, the compound of Formula I, or a pharmaceutically acceptable salt thereof, is administered over a period of 8 weeks. In some embodiments, the compound of Formula I, or a pharmaceutically acceptable salt thereof, is administered on day 1 and day 2 of each week.

When a compound of Formula I is administered multiple times a week, the dose may be administered on any day or combination of days within the week. For example, administration three times per week may include administration on days 1, 3, and 5; days 1, 2, and 3; 1, 3, and 5; and so on. Administration two days per week may include administration on days 1 and 2; days 1 and 3; days 1 and 4; days 1 and 5; days 1 and 6; days 1 and 7; and so on.

### Kits and Products

Some embodiments of the present disclosure relate to kits and products that include the compound of Formula I and/or at least on FGFR inhibitor. For example, the kit or product can include a package or container with a compound of Formula I. Such kits and products can further include a product insert or label with approved drug administration and indication information, including how to use the compound of Formula I in combination with an FGFR inhibitor that is separately provided. The kits can be used in the methods of treating cancer as described herein.

In some aspects, the kits or products can include both a compound of Formula I and at least one FGFR inhibitor. In some embodiments, the FGFR inhibitor is erdafitinib, for example. Such kits can include one or more containers or packages, which include one or both combination drugs together in a single container and/or package, or in separate packages/containers. In some instances, the two drugs are separately wrapped, but included in a single package, container or box. Such kits and products can further include a product insert or label with approved drug administration and indication information, including how to use the compound of Formula I in combination with an FGFR inhibitor. The kits can be used in the methods of treating cancer as described herein.

Some embodiments of the present disclosure relate to kits and products that include the compound of Formula 1 and/or at least on B-Raf inhibitor. For example, the kit or product can include a package or container with a compound of Formula I. Such kits and products can further include a product insert or label with approved drug administration and indication information, including how to use the compound of Formula 1 in combination with a B-Raf inhibitor that is separately provided. The kits can be used in the methods of treating cancer as described herein.

In some aspects, the kits or products can include both a compound of Formula 1 and at least one B-Raf inhibitor. In some embodiments, the B-Raf inhibitor is encorafenib, for example. Such kits can include one or more containers or packages, which include one or both combination drugs together in a single container and/or package, or in separate packages/containers. In some instances, the two drugs are separately wrapped, but included in a single package, container or box. Such kits and products can further include a product insert or label with approved drug administration and indication information, including how to use the compound of Formula 1 in combination with a B-Raf inhibitor. The kits can be used in the methods of treating cancer as described herein.

Some embodiments of the present disclosure relate to kits and products that include the compound of Formula I and/or at least one MEK inhibitor. For example, the kit or product can include a package or container with a compound of Formula I. Such kits and products can further include a product insert or label with approved drug administration and indication information, including how to use the compound of Formula 1 in combination with a MEK inhibitor that is separately provided. The kits can be used in the methods of treating cancer as described herein.

In some aspects, the kits or products can include both a compound of Formula 1 and at least one MEK inhibitor. In some embodiments, the MEK inhibitor is trametinib or binimetinib, for example. Such kits can include one or more containers or packages, which include one or both combination drugs together in a single container and/or package, or in separate packages/containers. In some instances, the two drugs are separately wrapped, but included in a single package, container or box. Such kits and products can further include a product insert or label with approved drug administration and indication information, including how to use the compound of Formula 1 in combination with a MEK inhibitor. The kits can be used in the methods of treating cancer as described herein.

Some embodiments of the present disclosure relate to kits and products that include the compound of Formula I and/or at least on MET inhibitor. For example, the kit or product can include a package or container with a compound of Formula I. Such kits and products can further include a product insert or label with approved drug administration and indication information, including how to use the compound of Formula I in combination with a MET inhibitor that is separately provided. The kits can be used in the methods of treating cancer as described herein.

In some aspects, the kits or products can include both a compound of Formula I and at least one MET inhibitor. In some embodiments, the MET inhibitor is crizotinib, tepotinib, savolitinib, cabozantinib, or tivantinib, for example. Such kits can include one or more containers or packages, which include one or both combination drugs together in a single container and/or package, or in separate packages/containers. In some instances, the two drugs are separately wrapped, but included in a single package, container or box. Such kits and products can further include a product insert or label with approved drug administration and indication information, including how to use the compound of Formula I in combination with a MET inhibitor. The kits can be used in the methods of treating cancer as described herein

### EXAMPLES

### Example 1—Synergistic Combination of the Compound of Formula I and Inhibitors of FGFR

This Example demonstrates the synergistic combination of the compound of Formula I with inhibitors of FGFR.

### Combination Cellular Proliferation Assays

Cells (2000 cells per well) were plated onto 96-well plates in 100 μl cell culture medium. Cells were treated with the compound of Formula I and erdafitinib at concentrations varying from 0 to 10 μM by using the Tecan D300e Digital Dispenser combination matrix protocol. At day 5, 50 μl of CellTiter-Glo (CTG) reagent (Promega) was added and the plates were incubated for 10 minutes with gentle shaking. After 10 minutes of incubation, the luminescent signal was determined according to the provider's instructions (Promega) and combination data was generated by the standard HSA model using Combenefit software. The combination synergy was represented by positive numbers in the results table. The negative numbers represent antagonism of the combination.

### Results

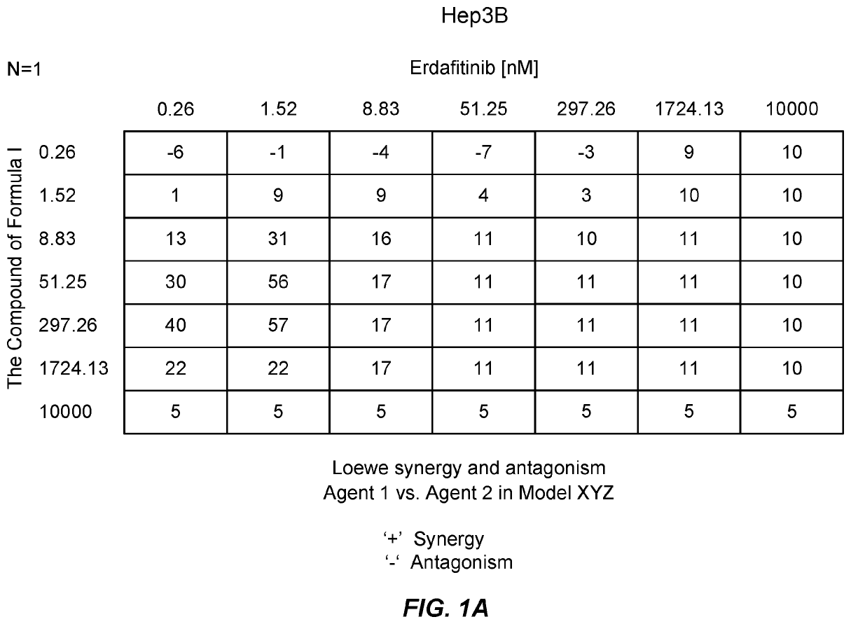

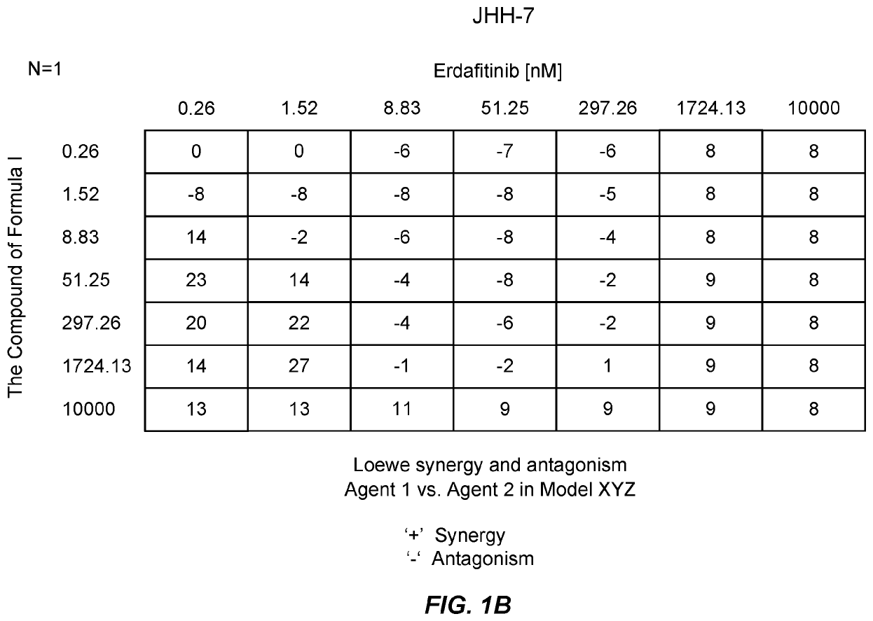

FIGS. 1A-1B show data indicating that the combinations of the compound of Formula I and FGFR inhibitor erdafitinib exhibit synergy in vitro. FIG. 1A shows 3D graphic synergy data in Hep3B cancer cell line using the combination of the compound of Formula I and erdafitinib. FIG. 1B shows 3D graphic synergy data in JHH-7 cancer cell line using the combination of the compound of Formula I and erdafitinib.

FIG. 1A and FIG. 1B show data indicating that the combinations of the compound of Formula I and FGFR inhibitor erdafitinib exhibit synergy in vitro. FIG. 1A shows synergy data in Hep3B cancer cell line using the combination of the compound of Formula I and erdafitinib. FIG. 1B shows synergy data in JHH-7 cancer cell line using the combination of the compound of Formula I and erdafitinib.

### Example 2—Combination Therapy of the Compound of Formula I and Erdafitinib in FGFR2 Amplified Hepatoma Carcinoma CDX Model KATO III Materials

The vehicle/control article, 100 mM acetic acid in deionized water, with pH adjustment to 4.8-5.0, was prepared and stored under ambient conditions throughout the 28-day administration in mice.

The test article of the compound of Formula I was freshly prepared in vehicle of 100 mM acetic buffer weekly and stored under ambient conditions. The combination agent erdafitinib was prepared in vehicle of 20% HP-β-CD and stored under 2-8° C.

Female Balb/c nude mice were purchased from the Beijing Vital River Laboratory Animal Technology Co., Ltd. Mice were between 6-8 weeks of age at the time of implantation. Mice were hosted at special pathogen-free (SPF) environment of vivarium facility and acclimated to their new environment for at least 3 days prior to initiation of any experiments according to IACUC protocol.

All procedures related to animal handling, care, and treatment in this study were performed according to guidelines approved by the Institutional Animal Care and Use Committee (IACUC) of WuXi AppTec. During the study, the care and use of animals were conducted in accordance with the regulations of the Association for Assessment and Accreditation of Laboratory Animal Care (AAALAC). In addition, all portions of this study performed at WuXi AppTec adhered to the study protocols approved by the study director and applicable standard operating procedures (SOPs).

### Preparation of Xenograft Model

The KATO-III cell line was human hepatoma carcinoma cells with the FGFR amplification. The KATO-III cell line was purchased from ATCC (ATCC® HTB-103™) 200 μL cell suspensions containing 5×10⁶ tumor cells mixed with 50% Matrigel were subcutaneously implanted into the right flank of mouse using a syringe. When tumor volumes reached a mean of 220 mm³ post subcutaneous implantation, tumor-bearing mice were randomized into different groups with 8 mice in each group. The randomization date was denoted as treatment day 0.

### Treatment

Treatment started on the day after randomization. The treatment start day was denoted as treatment day 1. Mice were dosed by oral administration of vehicle control solution, the compound of Formula I alone at 10 mg/kg BID, and erdafitinib alone at 10 mg/kg QD. One additional group received the combination treatment of the compound of Formula I and erdafitinib, with dosing of the compound of Formula I at 10 mg/kg BID and dosing of erdafitinib at 10 mg/kg QD. The dosing volume was 5 mL/kg and interval of BID regimen was 8 hours. Erdafitinib was dosed at one hour after the first BID dose of the compound of Formula I in the combination group. The study was terminated on treatment day 28 as defined in the study protocol.

### Results

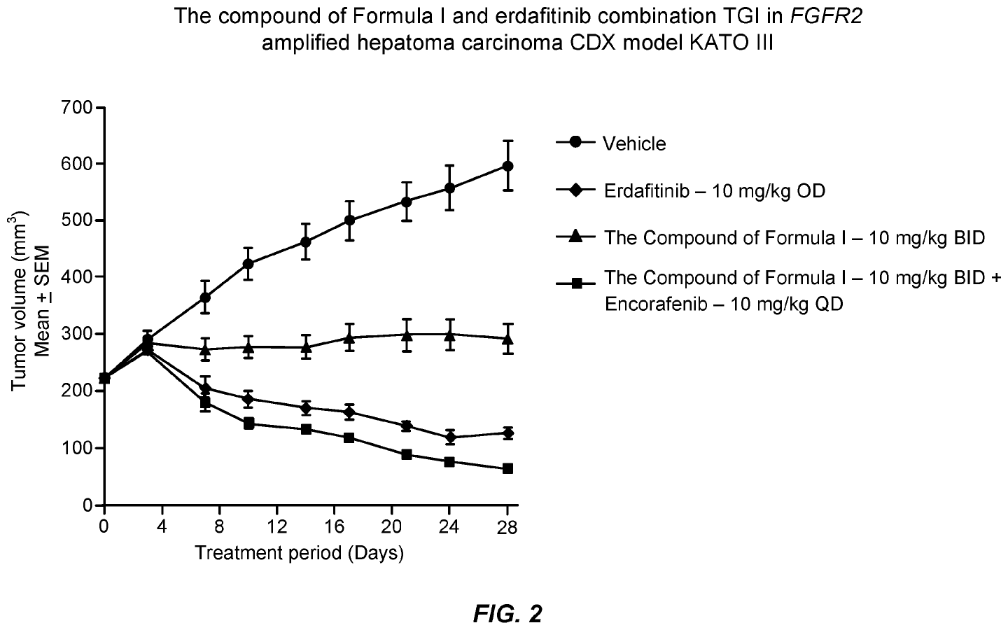

FIG. 2 shows a graph of tumor volume over a period of treatment time with the compound of Formula I alone, erdafitinib alone, and the combination of the compound of Formula I and erdafitinib in hepatoma carcinoma CDX model KATO III. No significant body weight change was observed in the control and treatment groups.

### Conclusion

As shown in FIG. 2, the combination of the compound of Formula I and erdafitinib demonstrated superior tumor growth inhibition relative to treatment with the compound of Formula I alone or treatment with erdafitinib alone in FGFR2 amplified hepatoma carcinoma CDX model KATO III.

### Example 3—Combination Therapy of the Compound of Formula I and Erdafitinib in FGFR2 Amplified Gastric Cancer CDX SNU-16

### Materials

The vehicle/control article, 100 mM acetic acid in deionized water, with pH adjustment to 4.8-5.0, was prepared and stored under ambient conditions throughout the 28-day administration in mice.

The test article of the compound of Formula I was freshly prepared in vehicle of 100 mM acetic buffer weekly and stored under ambient conditions. The combination agent erdafitinib was freshly prepared in vehicle of 20% HP-β-CD weekly and stored at 2-8° C.

Female Balb/c nude mice were purchased from the Beijing Vital River Laboratory Animal Technology Co., Ltd. Mice were between 6-8 weeks of age at the time of implantation. Mice were hosted at special pathogen-free (SPF) environment of vivarium facility and acclimated to their new environment for at least 3 days prior to initiation of any experiments according to IACUC protocol.

All procedures related to animal handling, care, and treatment in this study were performed according to guidelines approved by the Institutional Animal Care and Use Committee (IACUC) of WuXi AppTec. During the study, the care and use of animals were conducted in accordance with the regulations of the Association for Assessment and Accreditation of Laboratory Animal Care (AAALAC). In addition, all portions of this study were performed at WuXi AppTec and adhered to the study protocols approved by the study director and applicable standard operating procedures (SOPs).

### Preparation of Xenograft Model

The SNU16 cell line was human gastric cancer cells with the FGFR amplification. The SNU16 cell line was purchased from ATCC (ATCC® CRL-1420™). 200 μL cell suspensions containing 5×10⁶ tumor cells mixed with 50% Matrigel were subcutaneously implanted into the right flank of mouse using a syringe. When tumor volumes reached a mean of 180 mm³ post subcutaneous implantation, tumor-bearing mice were randomized into different groups with 8 mice in each group. The randomization date was denoted as treatment day 0.

### Treatment

Treatment started on the day of randomization. The treatment start day was denoted as treatment day 0. Mice were dosed by oral administration of vehicle control solution, the compound of Formula I alone at 10 mg/kg BID, the compound of Formula I alone at 30 mg/kg QD, and erdafitinib alone at 10 mg/kg QD. Two additional groups received the combination treatment of the compound of Formula I and erdafitinib, with one group receiving the compound of Formula I at 10 mg/kg BID and erdafitinib at 10 mg/kg QD, and the other group receiving the compound of Formula I at 30 mg/kg QD and erdafitinib at 10 mg/kg QD. The dosing volume for each compound was 5 mL/kg and the interval of BID regimen was 8 hours. Erdafitinib was dosed one after the dosing of the compound of Formula I QD or the first BID dose of the compound of Formula I in the combination groups. The study was terminated on treatment day 28 as being defined in the study protocol.

### Results

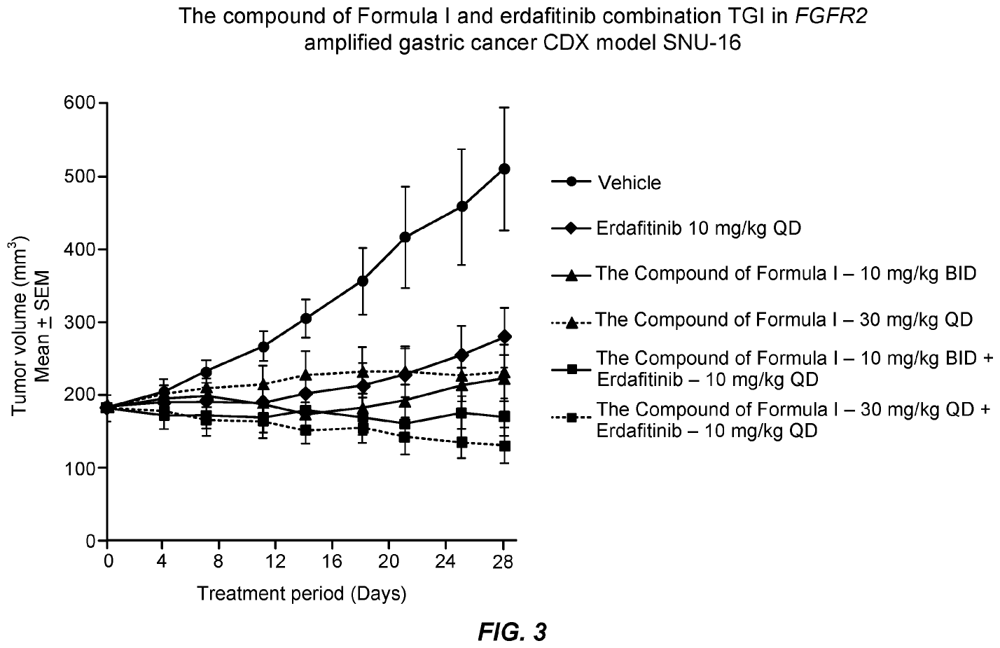

FIG. 3 shows a graph of tumor volume over a period of treatment time with the compound of Formula I alone, erdafitinib alone, and the combination of the compound of Formula I and erdafitinib in FGFR2 amplified gastric cancer CDX model SNU-16. No significant body weight change was observed in the control and treatment groups.

### Conclusion

As shown in FIG. 3, the combination of the compound of Formula I and erdafitinib demonstrated superior tumor growth inhibition relative to treatment with the compound of Formula I alone or treatment with erdafitinib alone in FGFR2 amplified gastric cancer CDX model SNU-16.

### Example 4—Combination Therapy of the Compound of Formula I and Erdafitinib in FGF19-FDFR4 Dependent Liver Cancer CDX Model Huh-7

### Materials

The test article of the compound of Formula I was freshly prepared in vehicle of 100 mM acetic buffer weekly and stored under ambient conditions. The combination agent erdafitinib was freshly prepared in vehicle of 20% HP-β-CD weekly and stored at 2-8° C.

Female Balb/c nude mice were purchased from the Beijing Vital River Laboratory Animal Technology Co., Ltd. Mice were between 6-8 weeks of age at the time of implantation. Mice were hosted in a special pathogen-free (SPF) environment of the vivarium facility and acclimated to their new environment for at least 3 days prior to initiation of any experiments according to IACUC protocol.

### Preparation of Xenograft Model

The Huh-7 cell line was human liver cancer cells with the FGFR overexpression. The Huh-7 cell line was purchased from the Japanese Collection of Research Bioresources Cell Bank (JCRB Cell Bank, JCRB0403). Huh-7 cells were cultured in medium containing Dulbecco's Modified Eagle Medium (DMEM) plus 10% Fetal Bovine Serum (FBS) and 1% Antibiotic-Antimycotic (AA) at 37° C. in an atmosphere of 5% CO₂ in air. The medium was renewed every 2 to 3 days and tumor cells were routinely sub-cultured at a confluence of 80-90% by trypsin-EDTA. The cells growing in an exponential growth phase were harvested and counted for inoculation. Huh-7 tumor cells were implanted into mice subcutaneously. 200 μL cell suspensions containing 5×10⁶ tumor cells mixed with 50% Matrigel were subcutaneously implanted into the right flank of mouse using a syringe. When the tumor volumes reached around 500-1000 mm³, tumor fragments (15-30 mm³) were harvested and then implanted subcutaneously in the right flanks of the mice using a 18 g trochar needle. Animal health and tumor growth were monitored daily. Tumor volume was measured twice a week by caliper when tumors were palpable and measurable. When tumor volumes reached a mean of 146 mm³ post subcutaneous implantation, tumor-bearing mice were randomized into different groups with 8 mice in each group. The randomization date was denoted as treatment day 0.

### Treatment

Treatment started on the day after randomization. The treatment start day was denoted as treatment day 1. Mice were dosed by oral administration of vehicle control solution, the compound of Formula I alone at 10 mg/kg BID, the compound of Formula I alone at 30 mg/kg QD, and erdafitinib alone at 10 mg/kg QD monotherapy treatment groups. Two additional groups received the combination treatment of the compound of Formula I and erdafitinib, with one group dosed with the compound of Formula I at 10 mg/kg BID and erdafitinib at 10 mg/kg QD, and the other group dosed with the compound of Formula I at 30 mg/kg QD and erdafitinib at 10 mg/kg QD. The dosing volume for each compound was 5 mL/kg and interval of BID regimen was 8 hours. Erdafitinib was dosed one hour after the dosing of the compound of Formula I QD or one hour after the first BID dose of the compound of Formula I in the combination groups. The study was terminated on treatment day 21.

### Results

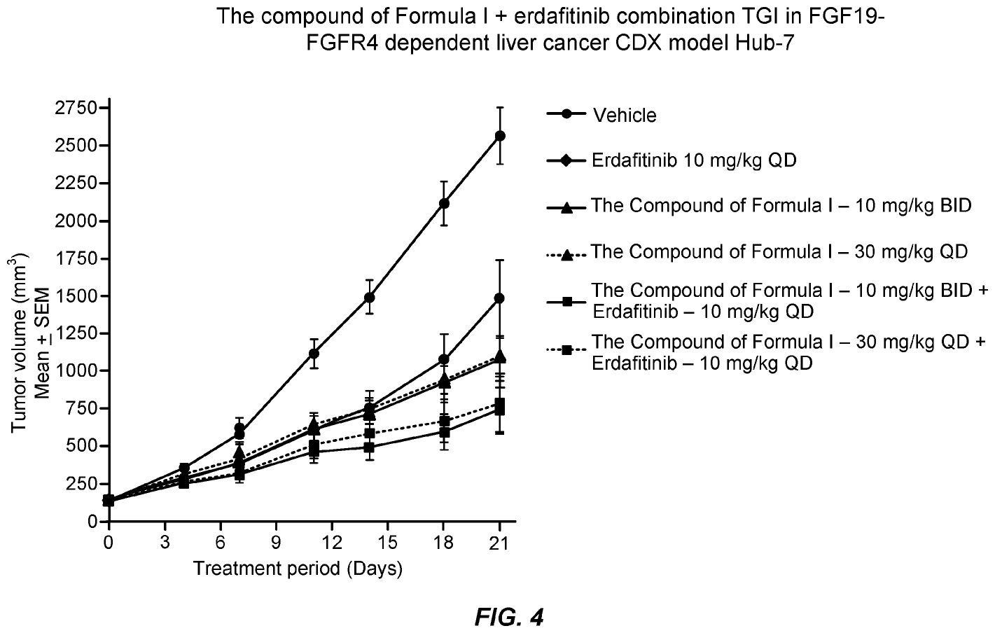

FIG. 4 shows a graph of tumor volume over a period of treatment time with the compound of Formula I alone, erdafitinib alone, and the combination of the compound of Formula I and erdafitinib in FGF19-FGFR4 dependent liver cancer CDX model Huh-7. No significant body weight change was observed in the control and treatment groups.

### Conclusion

As shown in FIG. 4, the combination of the compound of Formula I and erdafitinib demonstrated superior tumor growth inhibition relative to treatment with the compound of Formula I alone or treatment with erdafitinib alone in FGF19-FGFR4 dependent liver cancer CDX model Huh-7.

### Example 5—Synergistic Combination of the Compound of Formula I and Inhibitors of Class 1 Mutant B-Raf Proteins In Vitro

### Cellular Proliferation Assay

The cells (2000 cells per well) were plated onto 96-well plates in 100 μl cell culture medium and treated with the compound of Formula I alone or the compound of Formula I with fixed concentration of encorafenib. At day 5, 50 μl of CellTiter-Glo (CTG) reagent (Promega) was added and the plates were incubated for 10 minutes with gentle shaking. After 10 minutes incubation, the luminescent signal was determined according to the provider's instruction (Promega), and graph was plotted using Prism GraphPad.

### Combination Cellular Proliferation Assays

Cells (2000 cells per well) were plated onto 96-well plates in 100 μl cell culture medium. Cells were treated with the compound of Formula I and encorafenib at concentrations varying from 0 to 10 μM by using the Tecan D300e Digital Dispenser combination matrix protocol. At day 5, 50 μl of CellTiter-Glo (CTG) reagent (Promega) was added and the plates were incubated for 10 minutes with gentle shaking. After 10 minutes of incubation, the luminescent signal was determined according to the provider's instructions (Promega) and combination data was generated by the standard HSA model using Combenefit software. The combination synergy was represented by positive numbers in results table. The negative numbers represent antagonism of the combination.

Western Blotting for pERK and ERK

Cells were treated with compounds for 4 hours. After treatment, the cells were lysed on ice for 10 minutes with Thermo Fisher RIPA lysis buffer with protease and phosphatase inhibitors. The cells were centrifuged at 4° for 10 minutes with a microcentrifuge. The supernatant was transferred to pre-chilled microcentrifuge tube and protein concentration of the lysate was measured using BCA method. Cell lysate supernatants of equal-amount of proteins were used for immunoblotting against pERK and total ERK.

### Results

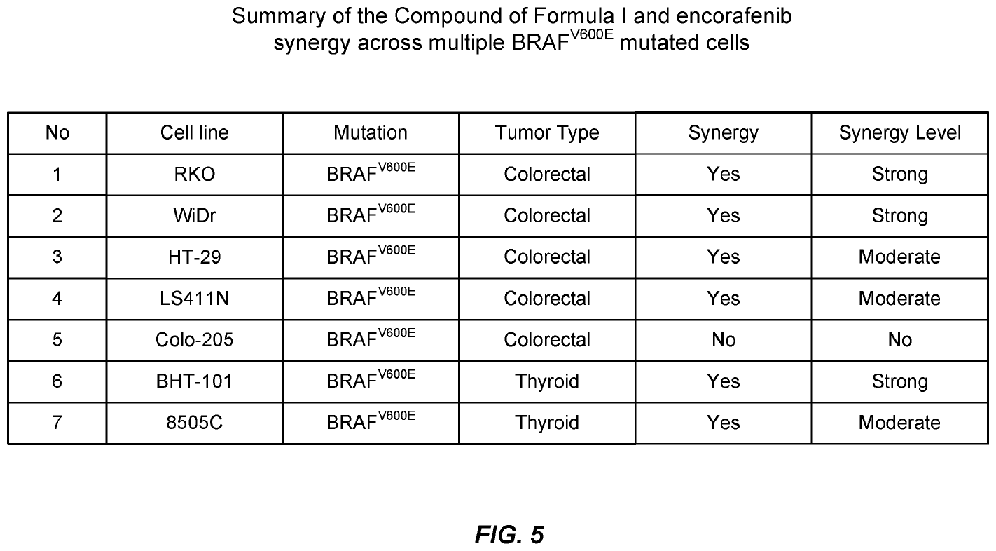

FIG. 5 shows data indicating that the combination of the compound of Formula I and encorafenib exhibits synergy across multiple BRAF V600E mutated cells.

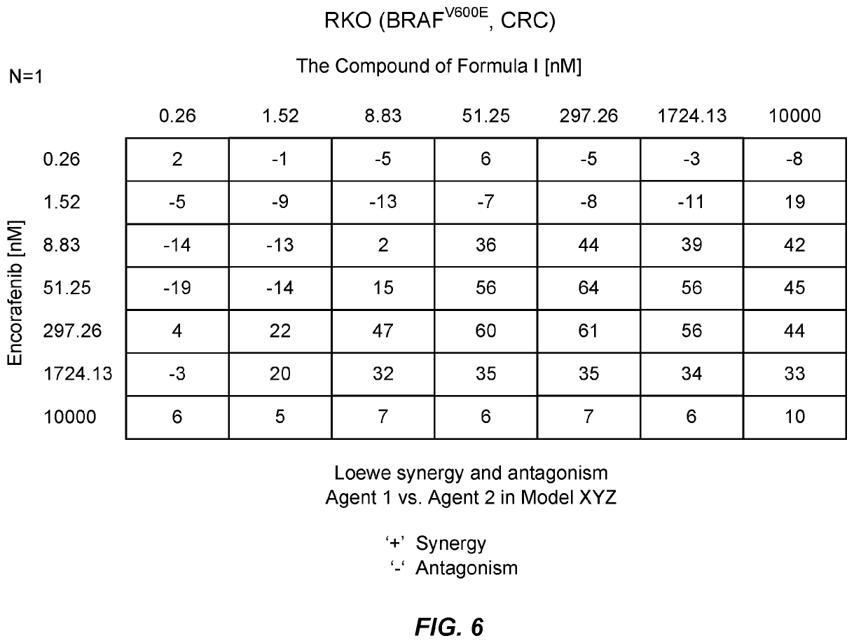

FIG. 6 shows a synergy data in RKO BRAF^{V600E} CRC cell line using the combination of the compound of Formula I and BRAF inhibitor encorafenib. This data indicates that there is a significant degree of synergy in the combination of the compound of Formula I and encorafenib.

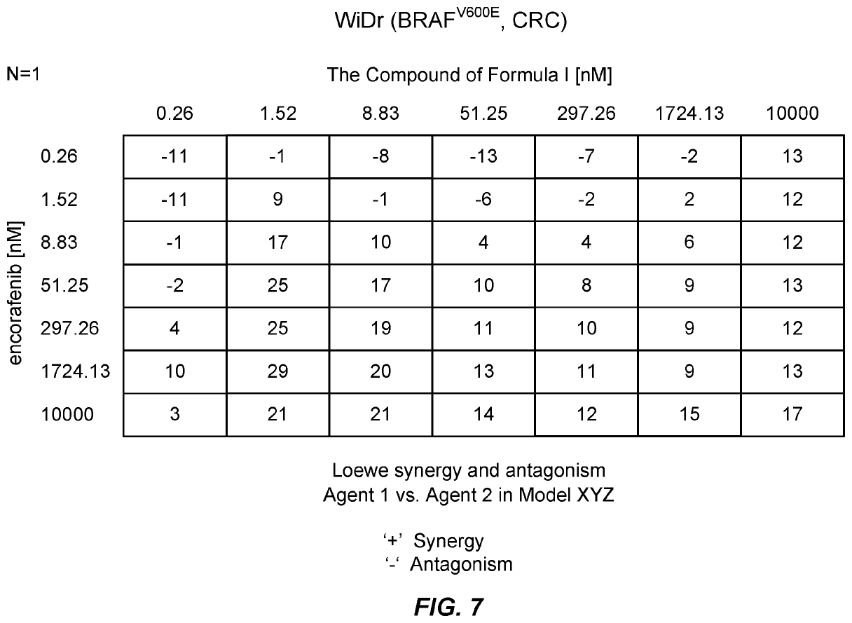

FIG. 7 shows a synergy data in WiDr BRAF^{V600E} CRC cell line using the combination of the compound of Formula I and BRAF inhibitor encorafenib. This data indicates that there is a significant degree of synergy in the combination of the compound of Formula I and encorafenib.

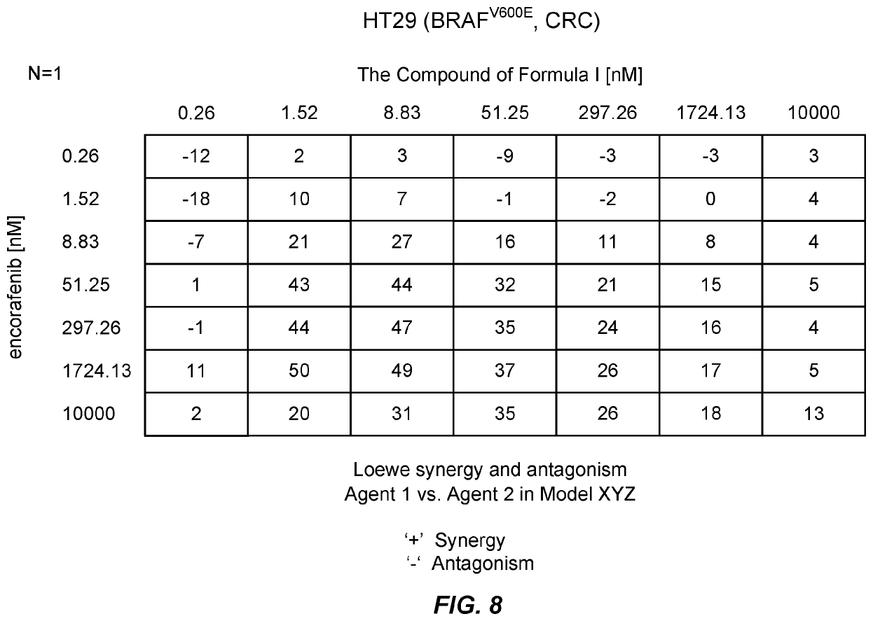

FIG. 8 shows a synergy data in HT29 BRAF^{V600E} CRC cell line using the combination of the compound of Formula I and BRAF inhibitor encorafenib. This data indicates that there is a significant degree of synergy in the combination of the compound of Formula I and encorafenib.

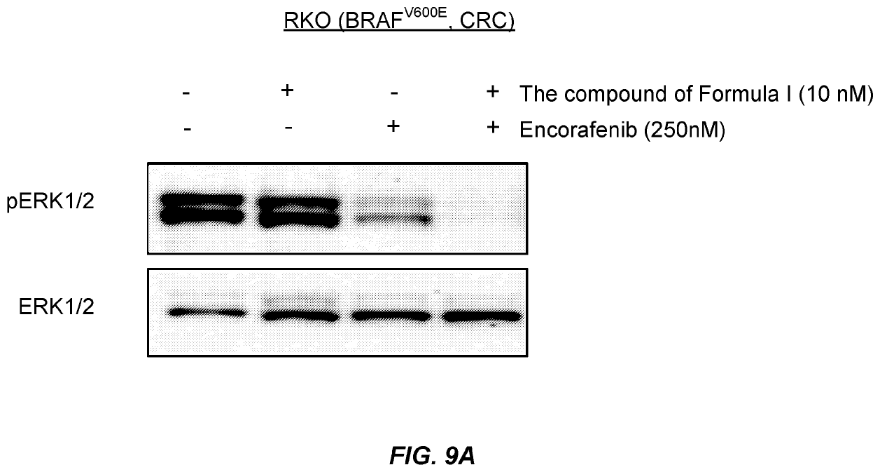

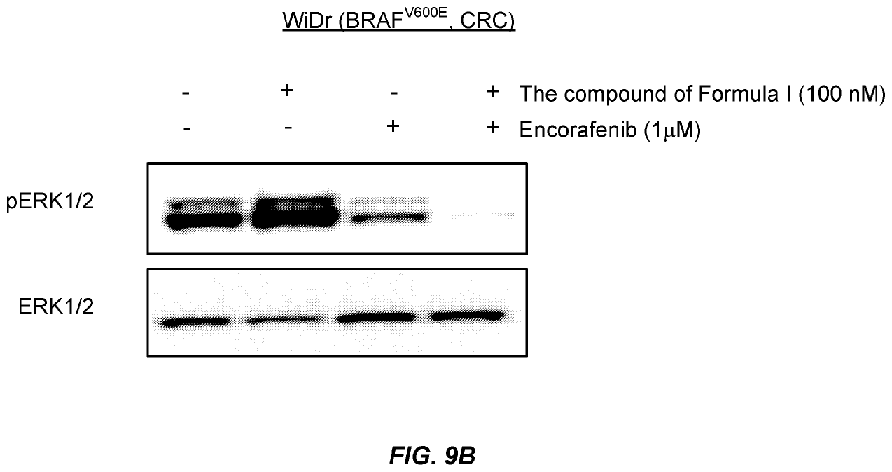

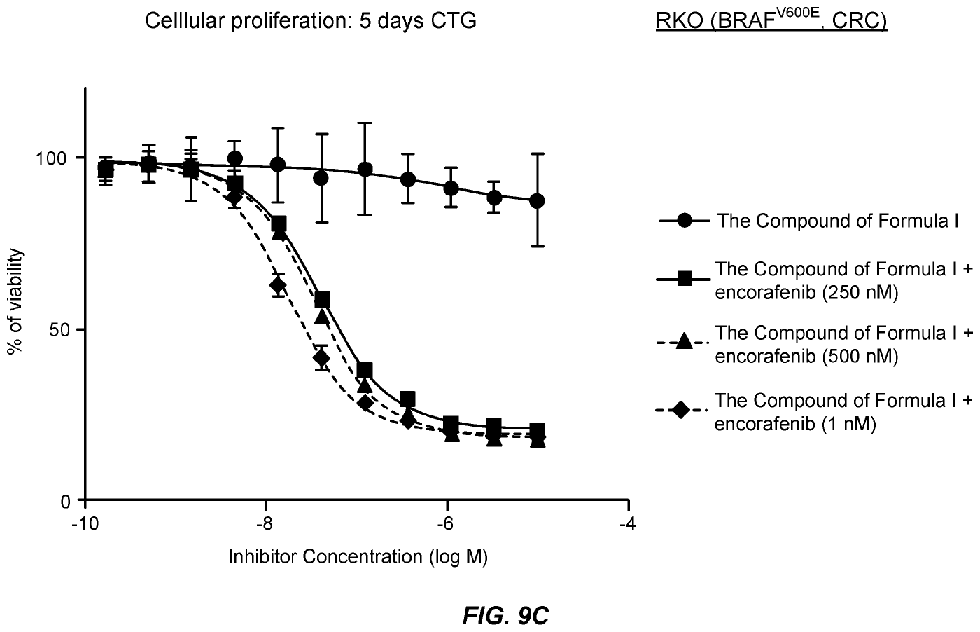

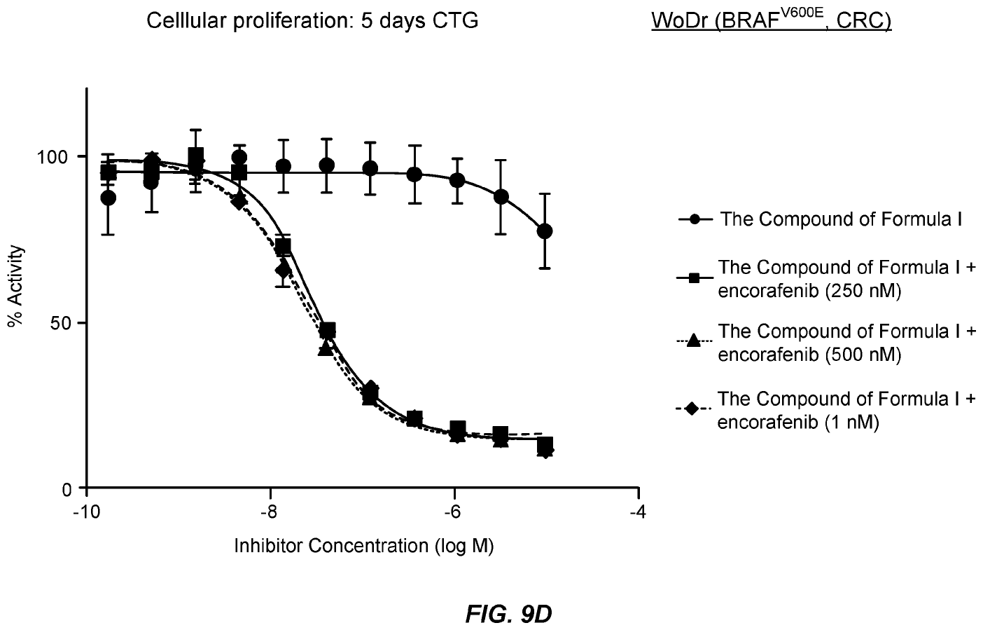

FIG. 9A shows a gel indicating a robust inhibition of ERK1/2 phosphorylation in the RKO colorectal cancer cell line. FIG. 9B shows a gel indicating a robust inhibition of ERK1/2 phosphorylation in the WiDr colorectal cancer cell line. FIG. 9C shows a plot of the antiproliferation effect of the compound of Formula I alone or the compound of Formula I combined with encorafenib in the RKO colorectal cancer cell line. FIG. 9D shows a plot of antiproliferation effect of the compound of Formula I or the compound of Formula I combined with encorafenib in the WiDr colorectal cancer cell line. FIG. 9A-9B indicate a robust inhibition of pERK1/2 using the combination of the compound of Formula I and encorafenib. FIG. 9C-9D suggest combination of the compound of Formula I and encorafenib increased inhibitory activity of the compound of Formula I.

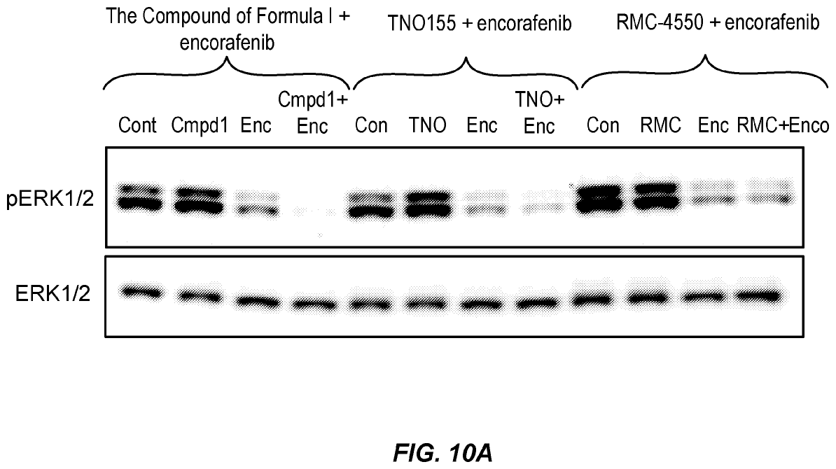

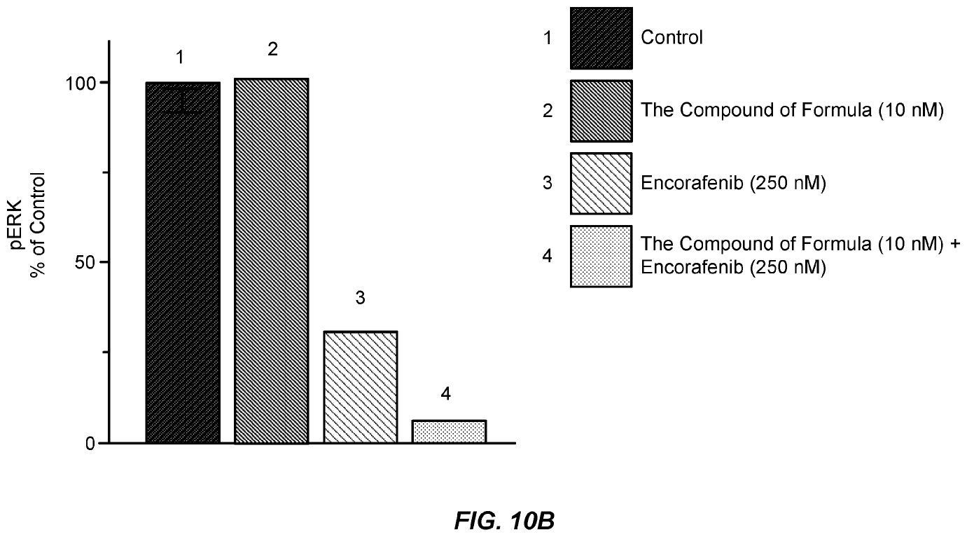

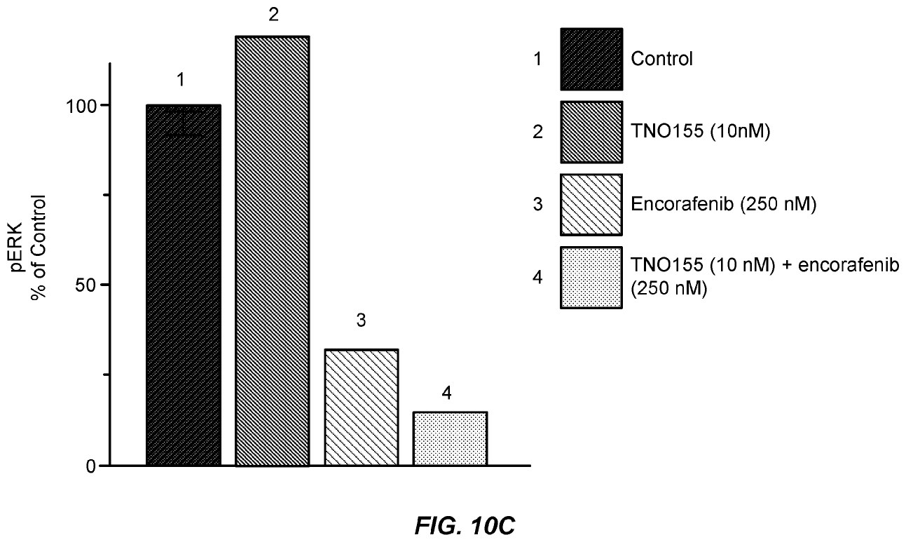

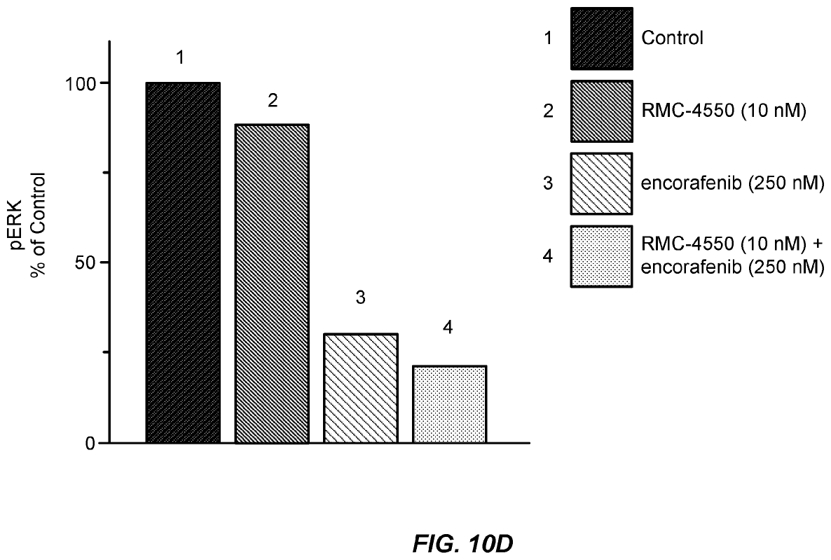

FIG. 10A-10D show a comparative study of the efficacy of combinations of SHP2 inhibitors with encorafenib in RKO colorectal cancer cell line. FIG. 10A shows a gel comparing inhibition of ERK1/2 phosphorylation in the RKO colorectal cancer cell line with combinations: the compound of Formula I+encorafenib; TNO155+encorafenib; and RMC-4550+encorafenib. FIG. 10B shows a bar graph of pERK as a percentage of control for 1. Control; 2. (the compound of Formula I); 3. encorafenib; and 4. (the compound of Formula I)+encorafenib. FIG. 10C shows a bar graph of pERK as a percentage of control for 1. Control; 2. TNO155; 3. encorafenib; and 4. TNO155+encorafenib. FIG. 10D shows a bar graph of pERK as a percentage of control for 1. Control; 2. RMC-4550; 3. encorafenib; and 4. RMC-4550+encorafenib. As indicated in FIG. 10A-10D, inhibition of ERK1/2 phosphorylation is most effective with the combination of SHP2 inhibitor compound of Formula I and encorafenib.

### Example 6—Combination Therapy of the Compound of Formula I and Encorafenib in BRAF^{V600E} Mutant CRC PDX Model CR0029

### Materials

The vehicle/control article, 100 mM acetic acid in deionized water, with pH adjustment to 4.8-5.0, was prepared and stored under ambient conditions throughout the 28-day administration in mice.

The test article of the compound of Formula I was freshly prepared in vehicle of 100 mM acetic buffer weekly and stored under ambient conditions. The combination agent encorafenib was freshly prepared in vehicle of 0.5% CMC and 0.5% Tween 80 weekly and stored at 2-8° C.

Female Balb/c nude mice were purchased from the SPF (Beijing) Laboratory Animal Technology Co, Ltd. Mice were between 7-9 weeks of age at the time of implantation. Mice were hosted at special pathogen-free (SPF) environment of vivarium facility and acclimated to their new environment for at least 3 days prior to initiation of any experiments according to IACUC protocol.

All procedures related to animal handling, care, and treatment in this study were performed according to guidelines approved by the Institutional Animal Care and Use Committee (IACUC) of Crown Bioscience (Taicang, China). During the study, the care and use of animals were conducted in accordance with the regulations of the Association for Assessment and Accreditation of Laboratory Animal Care (AAALAC). In addition, all portions of this study performed at Crown Bioscience (Taicang, China) adhered to the study protocols approved by the study director and applicable standard operating procedures (SOPs)

### Preparation of Xenograft Model

CR0029 PDX model was established for preclinical efficacy study at CrownBio. This PDX model was derived from a female Chinese CRC patient. A BRAF^{V600E} mutation in the PDX model CR0029 was confirmed by both RNA sequencing and Exome sequencing. Mouse skin was cleaned with appropriate surgical scrub and iodophor over the right flank. Tumor fragments (2-3 mm in diameter) harvested from the PDX model were implanted subcutaneously in the right flanks of female Balb/c nude mice using a 18 g trochar needle.

Animal health and tumor growth were monitored daily. Tumor volume was measured twice a week by caliper when tumors were palpable and measurable. When tumor volumes reached a mean of near 141 mm³ (range of 110-176 mm³), tumor-bearing mice were randomized into 7 different groups with 8 mice in each group. The randomization date was denoted as treatment day 0.

### Treatment

Treatment started on the day of randomization. The treatment start day was denoted as treatment day 0. Mice were dosed by oral administration of vehicle control solution, the compound of Formula I alone at 10 mg/kg BID, and encorafenib alone at 90 mg/kg QD. One additional group received the combination treatment, with dosing of the compound of Formula I at 10 mg/kg BID and dosing of encorafenib at 90 mg/kg QD. The dosing volume for each compound was 5 mL/kg and interval of BID regimen was 8 hours. Encorafenib was dosed one hour after dosing of the compound of Formula I in the combination group. The study was terminated on treatment day 28, as defined in the study protocol.

### Results

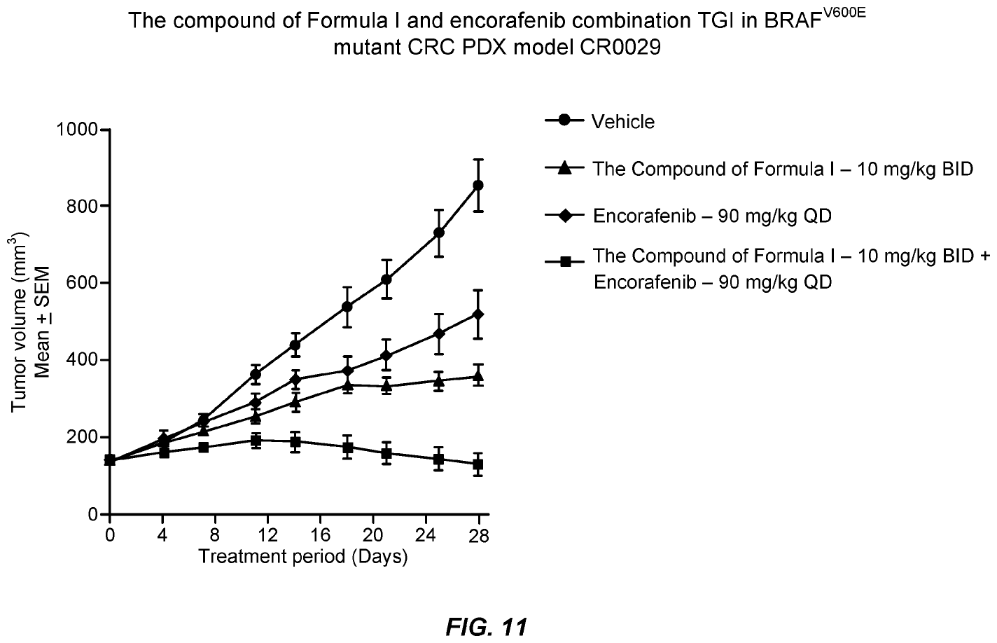

FIG. 11 shows a graph of tumor volume over a period of treatment time with the compound of Formula I alone, encorafenib alone, and the combination of the compound of Formula I and encorafenib in BRAF^{V600E} mutant CRC PDX model CR0029. No significant body weight change was observed in the control and treatment groups.

### Conclusion

As shown in FIG. 11, the combination of the compound of Formula I and encorafenib demonstrated superior tumor growth inhibition relative to treatment with the compound of Formula I alone or treatment with encorafenib alone in BRAF^{V600E} mutant CRC PDX model CR0029.

### Example 7—Combination Therapy of the Compound of Formula I and Encorafenib in BRAF^{V600E} Mutant CRC PDX Model CR004

### Materials

The vehicle/control article, 100 mM acetic acid in deionized water, with pH adjustment to 4.8-5.0, was prepared and stored under ambient conditions throughout the 28-day administration in mice.

The test article of the compound of Formula I was freshly prepared in vehicle of 100 mM acetic buffer weekly and stored under ambient conditions. The combination agent encorafenib was freshly prepared in vehicle of 0.5% CMC and 0.5% Tween 80 weekly and stored at 2-8° C.

Female Balb/c nude mice were purchased from the SPF (Beijing) Laboratory Animal Technology Co, Ltd. Mice were between 9-11 weeks of age at the time of implantation. Mice were hosted at special pathogen-free (SPF) environment of vivarium facility and acclimated to their new environment for at least 3 days prior to initiation of any experiments according to IACUC protocol.

All procedures related to animal handling, care, and treatment in this study were performed according to guidelines approved by the Institutional Animal Care and Use Committee (IACUC) of Crown Bioscience (Beijing, China). During the study, the care and use of animals were conducted in accordance with the regulations of the Association for Assessment and Accreditation of Laboratory Animal Care (AAALAC). In addition, all portions of this study performed at Crown Bioscience (Beijing, China) adhered to the study protocols approved by the study director and applicable standard operating procedures (SOPs).

### Preparation of PDX

CR0004 PDX model was established for preclinical efficacy study at CrownBio. This PDX model was derived from a 73-year-old male Chinese CRC patient. A BRAFV600E mutation in the PDX model CR0004 was confirmed by both RNA sequencing and exome sequencing. Mouse skin was cleaned with appropriate surgical scrub and iodophor over the right flank. Tumor fragments (2-3 mm in diameter) harvested from the PDX model were implanted subcutaneously in the right flanks of female Balb/c nude mice using a 18 g trochar needle. When mean tumor sizes reached 141 mm3 (range of 121-180 mm3), tumor-bearing mice were randomly divided into 6 study groups with 8 mice per group.

### Treatment

Treatment started on the day of randomization. The treatment start day was denoted as treatment day 0. Mice were dosed by oral administration of vehicle control solution, the compound of Formula I alone at 10 mg/kg BID and encorafenib alone at 90 mg/kg QD. One additional group received the combination treatment, with dosing of the compound of Formula I at 10 mg/kg BID and dosing of encorafenib at 90 mg/kg QD. The dosing volume for each compound was 5 mL/kg and interval of BID regimen was 8 hours. Encorafenib was dosed one hour after the dosing of the compound of Formula I in the combination group. The study was terminated on treatment day 28 as defined in the study protocol.

### Results

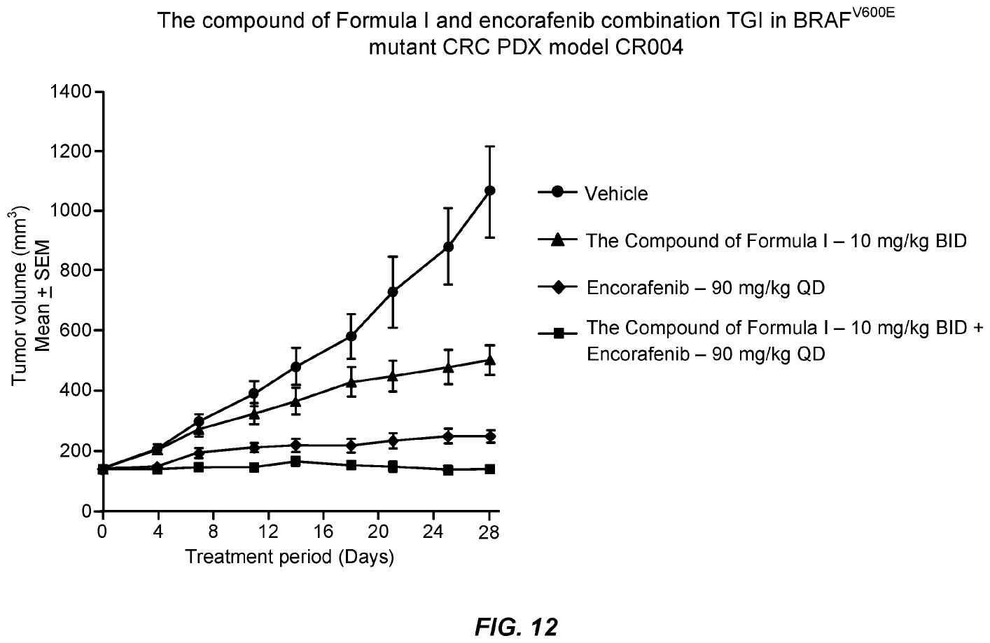

FIG. 12 shows a graph of tumor volume over a period of treatment time with the compound of Formula I alone, encorafenib alone, and the combination of the compound of Formula I and encorafenib in BRAF^{V600E} mutant CRC PDX model CR004. No significant body weight change was observed in the control and treatment groups.

### Conclusion

As shown in FIG. 12, the combination of the compound of Formula I and encorafenib demonstrated superior tumor growth inhibition relative to treatment with the compound of Formula I alone or treatment with encorafenib alone in BRAF^{V600E} mutant CRC PDX model CR004.

### Example 8—Combination Therapy of the Compound of Formula I and Encorafenib in BRAF^{V600E} Mutant CRC CDX model WiDr

### Materials

The vehicle/control article, 100 mM acetic acid in deionized water, with pH adjustment to 4.8-5.0, was prepared and stored under ambient conditions throughout the 28-day administration in mice.

The test article of the compound of Formula I was freshly prepared in vehicle of 100 mM acetic buffer weekly and stored under ambient conditions. The combination agent, encorafenib, was freshly prepared in vehicle of 0.5% CMC and 0.5% Tween 80 weekly and stored at 2-8° C.

Female Balb/c nude mice were purchased from the Beijing Vital River Laboratory Animal Technology Co., Ltd. Mice were between 6-8 weeks of age at the time of implantation. Mice were hosted at special pathogen-free (SPF) environment of vivarium facility and acclimated to their new environment for at least 3 days prior to initiation of any experiments according to IACUC protocol.

All procedures related to animal handling, care, and treatment in this study were performed according to guidelines approved by the Institutional Animal Care and Use Committee (IACUC) of WuXi AppTec. During the study, the care and use of animals were conducted in accordance with the regulations of the Association for Assessment and Accreditation of Laboratory Animal Care (AAALAC). In addition, all portions of this study performed at WuXi AppTec adhered to the study protocols approved by the study director and applicable standard operating procedures (SOPs).

### Preparation of Xenograft Model

WiDr was a human CRC tumor cell line that harbored a BRAFV600E mutation. The WiDr cell line was purchased from the European Collection of Authenticated Cell Cultures (ECACC, 85111501). WiDr cells were cultured in medium containing EEMEM (EBSS) plus 10% Fetal Bovine Serum (FBS), 2 mM Glutamine, and supplemented with 1% non-essential amino acids (NEAA) at 37° C. in an atmosphere of 5% CO₂ in air. The medium was renewed every 2 to 3 days and tumor cells were routinely sub-cultured at a confluence of 80-90% by trypsin-EDTA. The cells growing in an exponential growth phase were harvested and counted for inoculation.

WiDr tumor cells were implanted into mice subcutaneously. 200 μL cell suspensions containing 5×106 tumor cells were subcutaneously implanted into the right flank of mouse using a syringe. Animal health and tumor growth were monitored daily. Tumor volume was measured twice a week by caliper when tumors were palpable and measurable. When tumor volumes reached a mean of 189 mm3 (range of 139-240 mm3), tumor-bearing mice were randomized into different groups with 8 mice in each group. The randomization date was denoted as treatment day 0.

### Treatment

Treatment started on the day of randomization. The treatment start day was denoted as treatment day 0. Mice were dosed by oral administration of vehicle control solution, the compound of Formula I alone at 10 mg/kg BID, the compound of Formula I alone at 30 mg/kg QD and encorafenib alone at 90 mg/kg QD. Two additional groups received combination treatment of the compound of Formula I and encorafenib, with the first group dosed with the compound of Formula I at 10 mg/kg BID and encorafenib at 90 mg/kg QD, and the second group dosed with the compound of Formula I at 30 mg/kg QD and encorafenib at 90 mg/kg QD. The dosing volume for the compound of Formula I and encorafenib was 5 mL/kg and interval of BID regimen was 8 hours. Encorafenib was dosed one hour after the dosing of the compound of Formula 1 QD in the combination groups. The study was terminated on treatment day 28 as defined in the study protocol.

### Results

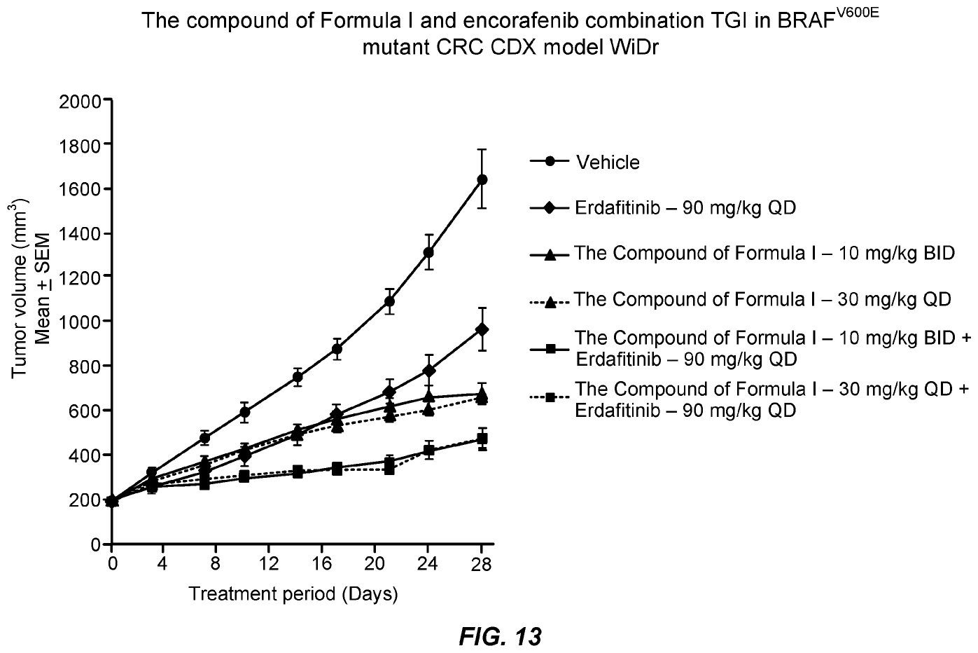

FIG. 13 shows a graph of tumor volume over a period of treatment time with the compound of Formula I alone, encorafenib alone, and the combination of the compound of Formula I and encorafenib in BRAF^{V600E} mutant CRC CDX model WiDr. No significant body weight change was observed in the control and treatment groups.

### Conclusion

As shown in FIG. 13, the combination of the compound of Formula I and encorafenib demonstrated superior tumor growth inhibition relative to treatment with the compound of Formula I alone or treatment with encorafenib alone in BRAF^{V600E} mutant CRC CDX model WiDr.

### Example 9—Combination Therapy of the Compound of Formula I and Encorafenib in BRAF^{V600E} Mutant CRC CDX Model HT-29

### Materials

The vehicle/control article, 100 mM acetic acid in deionized water, with pH adjustment to 4.8-5.0, was prepared and stored under ambient conditions throughout the 28-day administration in mice.

The test article Formula 1 was freshly prepared in vehicle of 100 mM acetic buffer weekly and stored under ambient conditions. The combination agent encorafenib was freshly prepared in vehicle of 0.5% CMC and 0.5% Tween 80 weekly and stored at 2-8° C.

Female Balb/c nude mice were purchased from the Beijing Vital River Laboratory Animal Technology Co., Ltd. Mice were hosted at special pathogen-free (SPF) environment of vivarium facility and acclimated to their new environment for at least 3 days prior to initiation of any experiments. Mice were between 6-8 weeks of age at the time of implantation.

All procedures related to animal handling, care, and treatment in this study were performed according to the protocols and guidelines approved by the Institutional Animal Care and Use Committee (IACUC) of GenenDesign. Animal facility and program is operated under the standard of Guide for the Care and Use of Laboratory Animals (NRC, 2011) and accredited by the Association for Assessment and Accreditation of Laboratory Animal Care (AAALAC). Specifically, all portions of this study performed at GenenDesign adhered to the study protocols reviewed and approved by IACUC and applicable standard operating procedures (SOPs).

### Preparation of Xenograft Model

HT-29 was a human CRC tumor cell line that harbored a BRAFV600E mutation. The HT-29 cell line was purchased from the American Type Culture Collection (ATCC® CRL-2577™). HT-29 cells were cultured in McCoy's 5a medium plus 10% fetal bovine serum (FBS) at 37° C. in an atmosphere of 5% C02 in air. The medium was renewed every 2 to 3 days and tumor cells were routinely sub-cultured at a confluence of 80-90% by trypsin-EDTA. The cells growing in an exponential growth phase were harvested and counted for inoculation.

HT-29 tumor cells were implanted into mice subcutaneously. 200 μL cell suspensions containing 2×106 tumor cells mixed with 50% Matrigel were subcutaneously implanted into the right flank of mouse using a syringe. Animal health and tumor growth were monitored daily. Tumor volume was measured twice a week by caliper when tumors were palpable and measurable. When tumor volumes reached a mean of near 200 mm3 (range of 146-259 mm3), tumor-bearing mice were randomized into different groups with 8 mice in each group. The randomization date was denoted as treatment day 0.

### Treatment

Treatment started on the day of randomization. The treatment start day was denoted as treatment day 0. Mice were dosed by oral administration of vehicle control solution, the compound of Formula I alone at 10 mg/kg BID, the compound of Formula I alone at 30 mg/kg QD and encorafenib alone at 90 mg/kg QD. Two additional groups received combination treatments of the compound of Formula I and encorafenib, with the first group dosed with the compound of Formula I at 10 mg/kg BID and encorafenib at 90 mg/kg QD, and the second group dosed with the compound of Formula I at 30 mg/kg QD and encorafenib at 90 mg/kg QD. The dosing volume for each compound was 5 mL/kg and interval of BID regimen was 8 hours. Encorafenib was dosed one hour after the dosing of the compound of Formula I in the combination groups. The study was terminated on treatment day 28, as defined in the study protocol.

### Results

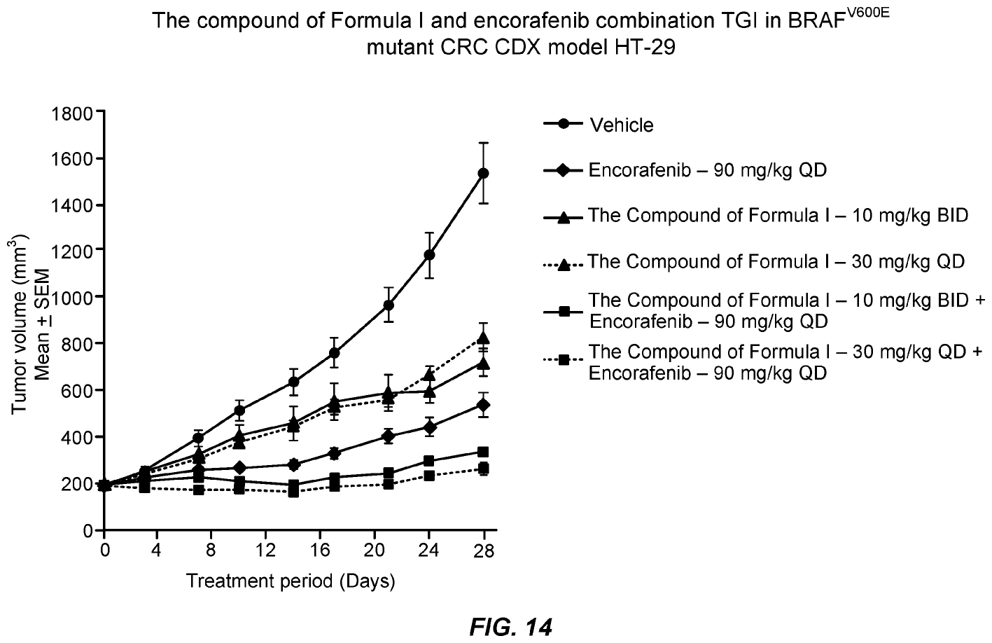

FIG. 14 shows a graph of tumor volume over a period of treatment time with the compound of Formula I alone, encorafenib alone, and the combination of the compound of Formula I and encorafenib in BRAF^{V600E} mutant CRC CDX model HT-29. No significant body weight change was observed in the control and treatment groups.

### Conclusion

As shown in FIG. 14, the combination of the compound of Formula I and encorafenib demonstrated superior tumor growth inhibition relative to treatment with the compound of Formula I alone or treatment with encorafenib alone in BRAF^{V600E} mutant CRC CDX model HT-29.

### Example 10—Combination Therapy of the Compound of Formula I and Encorafenib in BRAF^{V600E} Mutant Thyroid Carcinoma CDX Model BHT-101

### Materials

The vehicle/control article, 100 mM acetic acid in deionized water, with pH adjustment to 4.8-5.0, was prepared and stored under ambient conditions throughout the 20-day administration in mice.

The test article of the compound of Formula I was freshly prepared in vehicle of 100 mM acetic buffer weekly and stored under ambient conditions. The combination agent encorafenib was freshly prepared in vehicle of 0.5% CMC and 0.5% Tween 80 weekly and stored at 2-8° C.

Female Balb/c nude mice were purchased from Beijing Vital River Laboratory Animal Technology Co., Ltd. Mice were hosted at the special pathogen-free (SPF) environment of vivarium facility and acclimated to their new environment for at least 3 days prior to initiation of any experiments. Mice were between 6-8 weeks of age at the time of implantation.

All procedures related to animal handling, care, and treatment in this study were performed according to the protocols and guidelines approved by the Institutional Animal Care and Use Committee (IACUC) of GenenDesign. Animal facility and program is operated under the standard of Guide for the Care and Use of Laboratory Animals (NRC, 2011) and accredited by the Association for Assessment and Accreditation of Laboratory Animal Care (AAALAC). Specifically, all portions of this study performed at GenenDesign adhered to the study protocols reviewed and approved by IACUC and applicable standard operating procedures (SOPs).

### Preparation of Xenograft Model

BHT-101 was a human thyroid carcinoma cell line that harbored a BRAFV600E mutation. The BHT-101 cell line was purchased from the Cell Bank of Chinese Academy of Sciences (originally from DSMZ-German Collection of Microorganisms and Cell Cultures GmbH). BHT-101 cells were cultured in DMEM medium containing 20% Fetal Bovine Serum (FBS) and supplemented with 1× Glutamax solution and 1 mM sodium pyruvate at 37° C. in an atmosphere of 5% CO₂ in air. The medium was renewed every 2 to 3 days and tumor cells were routinely sub-cultured at a confluence of 80-90% by trypsin-EDTA. The cells growing in an exponential growth phase were harvested and counted for inoculation.

BHT-101 tumor cells were implanted into mice subcutaneously. 200 μL cell suspensions containing 2×106 tumor cells mixed with 50% Matrigel were subcutaneously implanted into the right flank of mouse using a syringe. Animal health and tumor growth were monitored daily. Tumor volume was measured twice a week by caliper when tumors were palpable and measurable. When tumor volumes reached a mean of 190 mm3 (range of 146-258 mm3), tumor-bearing mice were randomized into different groups with 8 mice in each group. The randomization date was denoted as treatment day 0.

### Treatment

Treatment started on the day of randomization. The treatment start day was denoted as treatment day 0. Mice were dosed by oral administration of vehicle control solution, the compound of Formula I alone at 10 mg/kg BID, the compound of Formula I alone at 30 mg/kg QD and encorafenib alone at 90 mg/kg QD. Two additional groups received combination treatments of the compound of Formula I and encorafenib, with the first group dosed with the compound of Formula I at 10 mg/kg BID and encorafenib at 90 mg/kg QD, and the second group dosed with the compound of Formula I at 30 mg/kg QD and encorafenib at 90 mg/kg QD. The dosing volume for each compound was 5 mL/kg and interval of BID regimen was 8 hours. Encorafenib was dosed one hour after the dosing of the compound of Formula I in combination groups. The study was terminated on treatment day 20, which was earlier than the original termination day as defined in the study protocol due to rapid tumor growth. Half of the tumors in the vehicle control group exceeded the tumor volume threshold per IACUC protocol (2,000 mm3) on treatment day 20.

### Results

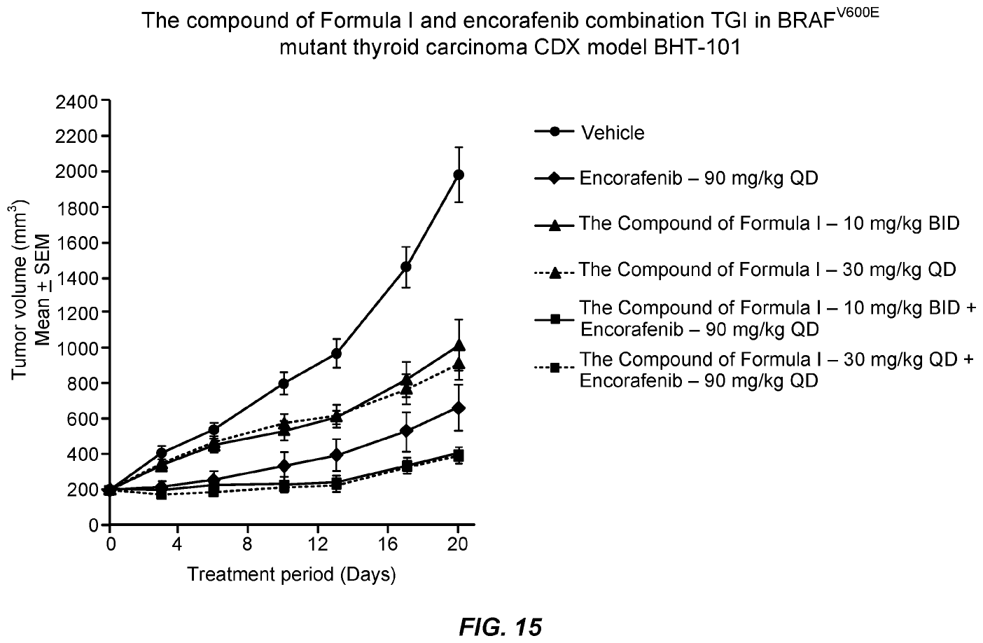

FIG. 15 shows a graph of tumor volume over a period of treatment time with the compound of Formula I alone, encorafenib alone, and the combination of the compound of Formula I and encorafenib in BRAF^{V600E} mutant thyroid carcinoma CDX model BHT-101. No significant body weight change was observed in the control and treatment groups.

### Conclusion

As shown in FIG. 15, the combination of the compound of Formula I and encorafenib demonstrated superior tumor growth inhibition relative to treatment with the compound of Formula I alone or treatment with encorafenib alone in BRAF^{V600E} mutant thyroid carcinoma CDX model BHT-101.

### Example 11—Combination Therapy of the Compound of Formula I and Encorafenib in BRAF^{V600E} Mutant CRC CDX Model RKO

### Materials

The vehicle/control article, 100 mM acetic acid in deionized water, with pH adjustment to 4.8-5.0, was prepared and stored under ambient conditions throughout the 16-day administration in mice.

The test article Formula 1 was freshly prepared in vehicle of 100 mM acetic buffer weekly and stored under ambient conditions. The combination agent encorafenib was freshly prepared in vehicle of 0.5% CMC and 0.5% Tween 80 weekly and stored at 2-8° C.

Female Balb/c nude mice were purchased from the Beijing Vital River Laboratory Animal Technology Co., Ltd. Mice were hosted at special pathogen-free (SPF) environment of vivarium facility and acclimated to their new environment for at least 3 days prior to initiation of any experiments. Mice were between 6-8 weeks of age at the time of implantation.

All procedures related to animal handling, care, and treatment in this study were performed according to the protocols and guidelines approved by the Institutional Animal Care and Use Committee (IACUC) of GenenDesign. Animal facility and program is operated under the standard of Guide for the Care and Use of Laboratory Animals (NRC, 2011) and accredited by the Association for Assessment and Accreditation of Laboratory Animal Care (AAALAC). Specifically, all portions of this study performed at GenenDesign adhered to the study protocols reviewed and approved by IACUC and applicable standard operating procedures (SOPs).

### Preparation of Xenograft Model

RKO was human CRC tumor cell line that harbored a BRAFV600E mutation. The RKO cell line was purchased from the American Type Culture Collection (ATCC® CRL-2577™). RKO cells were cultured in medium containing MEM plus 10% Fetal Bovine Serum (FBS) supplemented with non-essential amino acids at 37° C. in an atmosphere of 5% C02 in air. The medium was renewed every 2 to 3 days and tumor cells were routinely sub-cultured at a confluence of 80-90% by trypsin-EDTA. The cells growing in an exponential growth phase were harvested and counted for inoculation.

RKO tumor cells were implanted into mice subcutaneously. 200 μL cell suspensions containing 2×106 tumor cells mixed with 50% Matrigel were subcutaneously implanted into the right flank of mouse using a syringe. Animal health and tumor growth were monitored daily. Tumor volume was measured twice a week by caliper when tumors were palpable and measurable. When tumor volumes reached a mean of 217 mm3 (range of 163-262 mm3), tumor-bearing mice were randomized into different groups with 8 mice in each group. The randomization date was denoted as treatment day 0.

### Treatment

Treatment started on the day of randomization. The treatment start day was denoted as treatment day 0. Mice were dosed by oral administration of vehicle control solution, the compound of Formula I alone at 10 mg/kg BID, the compound of Formula I alone at 30 mg/kg QD and encorafenib alone at 90 mg/kg QD. Two additional groups received the combination treatments of the compound of Formula I, with the first group dosed with the compound of Formula I at 10 mg/kg BID and encorafenib at 90 mg/kg QD, and the second group dosed with the compound of Formula I at 30 mg/kg QD and encorafenib at 90 mg/kg QD. The dosing volume for each compound was 5 mL/kg and interval of BID regimen was 8 hours. Encorafenib was dosed one hour after the dosing of the compound of Formula I QD dose in the combination groups. The study was terminated on treatment day 16, which was earlier than the original termination day as defined in the study protocol due to rapid tumor growth. The majority of tumors in the vehicle control group exceeded the tumor volume threshold per IACUC protocol (2,000 mm3) on treatment day 16.

### Results

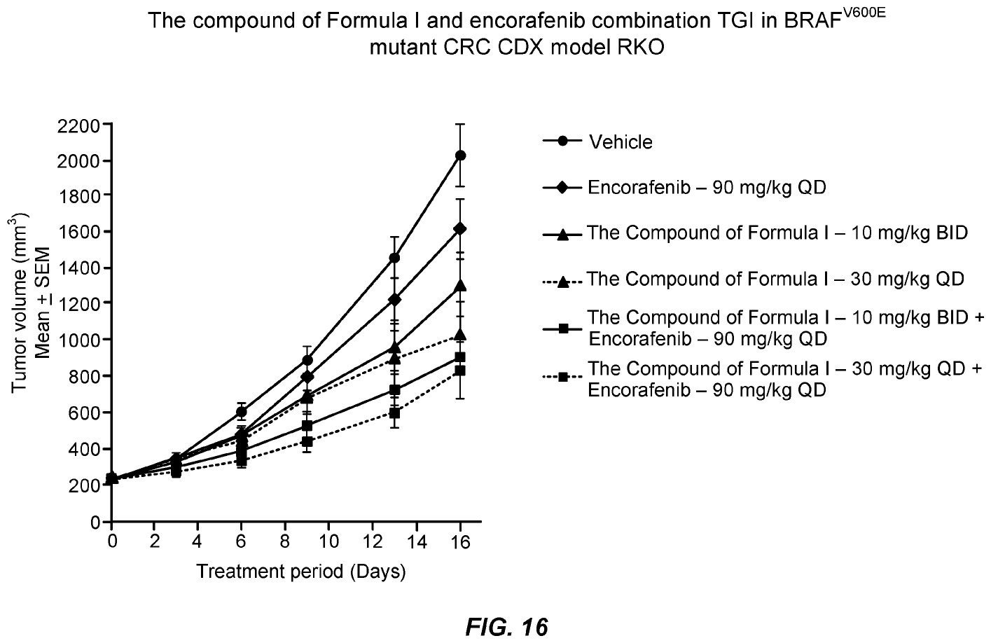

FIG. 16 shows a graph of tumor volume over a period of treatment time with the compound of Formula I alone, encorafenib alone, and the combination of the compound of Formula I and encorafenib in BRAF^{V600E} mutant CRC CDX model RKO. No significant body weight change was observed in the control and treatment groups.

### Conclusion

As shown in FIG. 16, the combination of the compound of Formula I and encorafenib demonstrated superior tumor growth inhibition relative to treatment with the compound of Formula I alone or treatment with encorafenib alone in BRAF^{V600E} mutant CRC CDX model RKO.

### Example 12—Synergistic Combination of the Compound of Formula I and Inhibitors of MEK

This Example demonstrates the synergistic combination of the compound of Formula I with inhibitors of MEK.

### Cellular Proliferation Assay

The cells (2000 cells per well) were plated onto 96-well plates in 100 μl cell culture medium and treated with the compounds of Formula I alone or the compound of Formula I with fixed concentration of trametinib or binimetinib. At day 5, 50 μl of CellTiter-Glo (CTG) reagent (Promega) was added and the plates were incubated for 10 minutes with gentle shaking. After 10 minutes incubation, the luminescent signal was determined according to the provider's instruction (Promega), and graph was plotted using Prism GraphPad.

### Combination Cellular Proliferation Assays

Cells (2000 cells per well) were plated onto 96-well plates in 100 μl cell culture medium. Cells were treated with the compound of Formula I and trametinib or binimetinib at concentrations varying from 0 to 10 μM by using the Tecan D300e Digital Dispenser combination matrix protocol. At day 5, 50 μl of CellTiter-Glo (CTG) reagent (Promega) was added and the plates were incubated for 10 minutes with gentle shaking. After 10 minutes of incubation, the luminescent signal was determined according to the provider's instructions (Promega) and combination data was generated by the standard HSA model using Combenefit software. The combination synergy was represented by positive numbers in results table. The negative numbers represent antagonism of the combination.

Western Blotting for pERK and ERK

NCI-H508 cells were treated with compounds for 4 hours. After treatment, the cells were lysed on ice for 10 minutes with Thermo Fisher RIPA lysis buffer with protease and phosphatase inhibitors. The cells were centrifuged at 4° for 10 minutes with a microcentrifuge. The supernatant was transferred to pre-chilled microcentrifuge tube and protein concentration of the lysate was measured using BCA method. Cell lysate supernatants of equal-amount of proteins were used for immunoblotting against pERK and total ERK.

MeWo cells were treated with compounds for 4 hours. After treatment, the cells were lysed on ice for 10 minutes with Thermo Fisher RIPA lysis buffer with protease and phosphatase inhibitors. The cells were centrifuged at 4° for 10 minutes with a microcentrifuge. The supernatant was transferred to pre-chilled microcentrifuge tube and protein concentration of the lysate was measured using BCA method. Cell lysate supernatants of equal-amount of proteins were used for immunoblotting against pERK and total ERK.

### Results

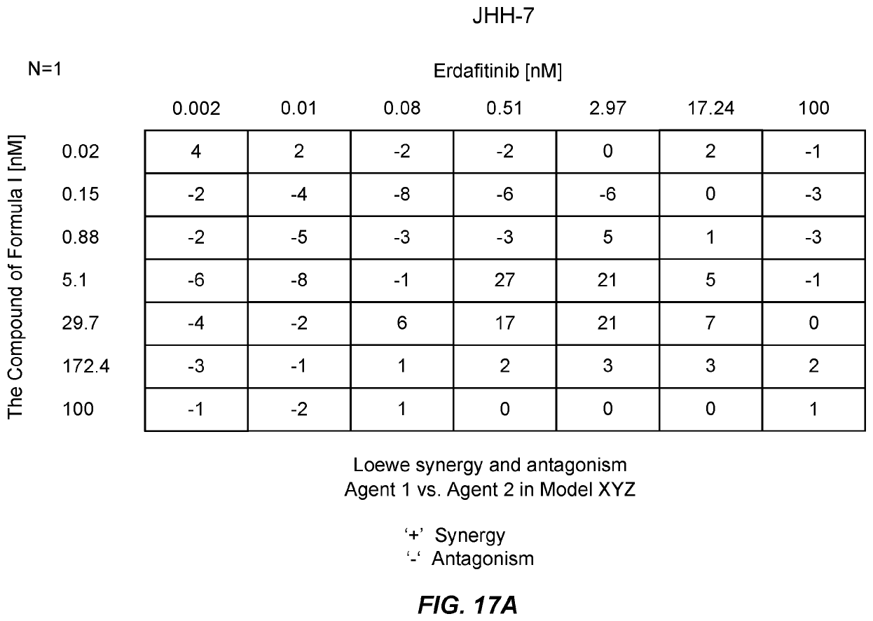

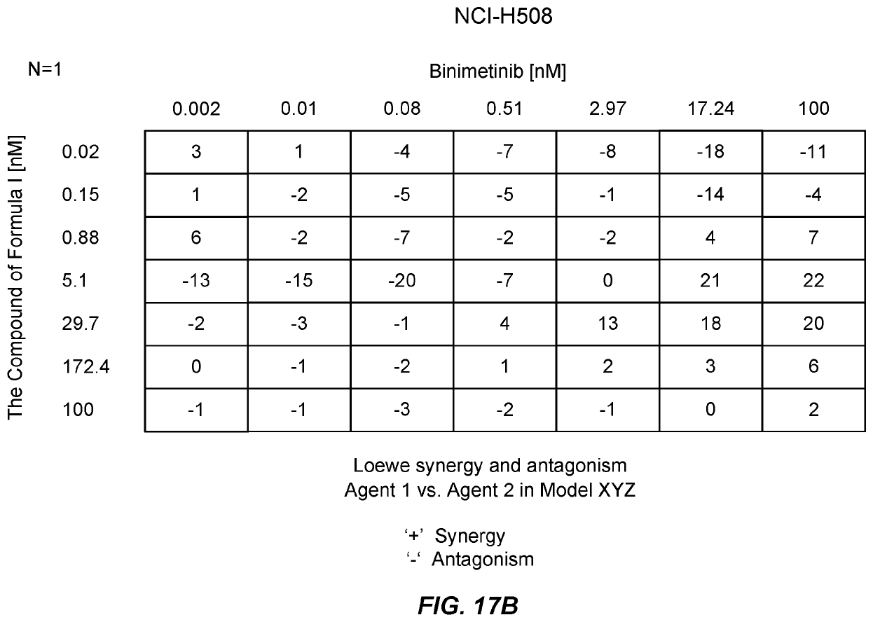

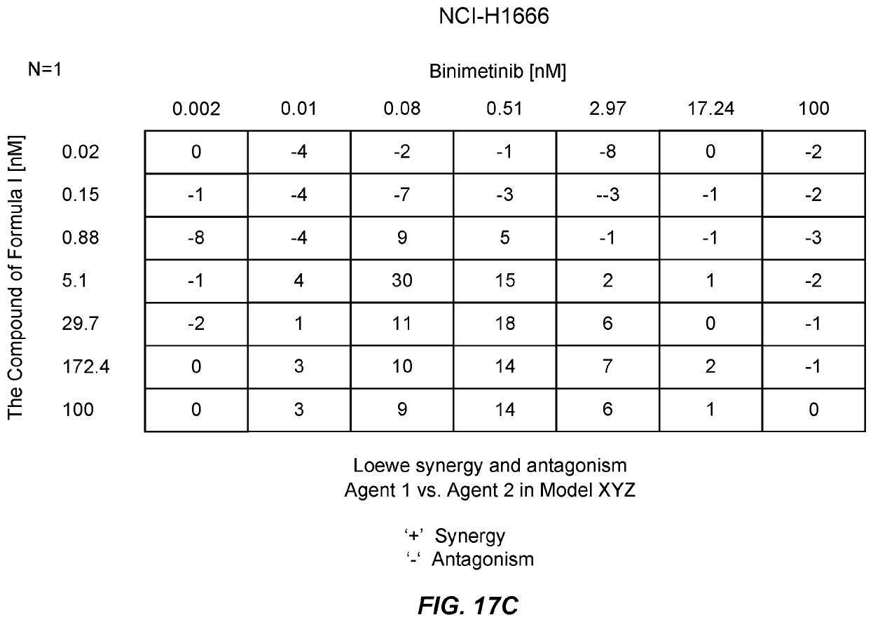

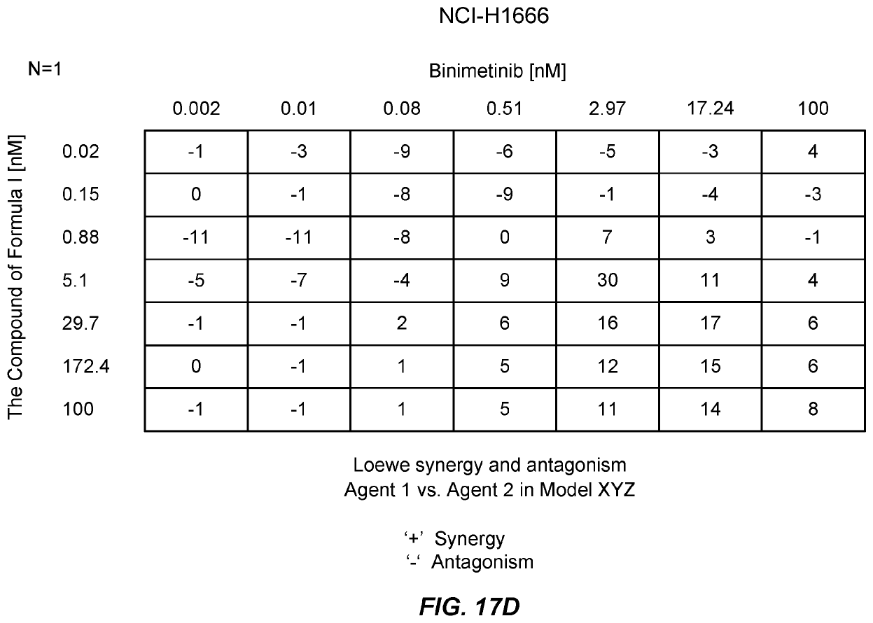

FIG. 17A shows synergy data in NCI-H508 cancer cell line using the combination of the compound of Formula I and trametinib. FIG. 17B shows synergy data in NCI-H508 cancer cell line using the combination of the compound of Formula I and binimetinib. FIG. 17C graphic synergy data in NCI-H1666 cancer cell line using the combination of the compound of Formula I and trametinib. FIG. 17D shows synergy data in NCI-H1666 cancer cell line using the combination of the compound of Formula I and binimetinib.

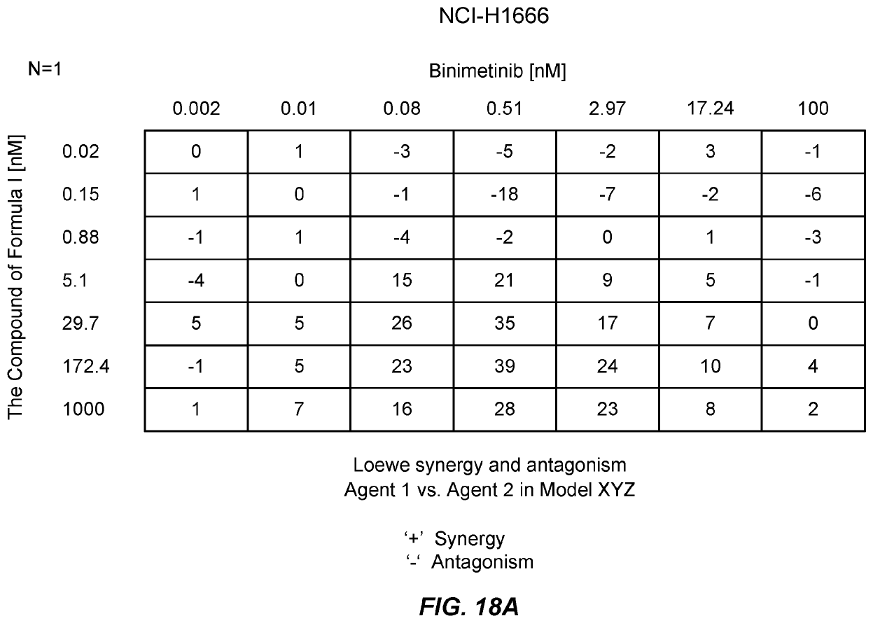

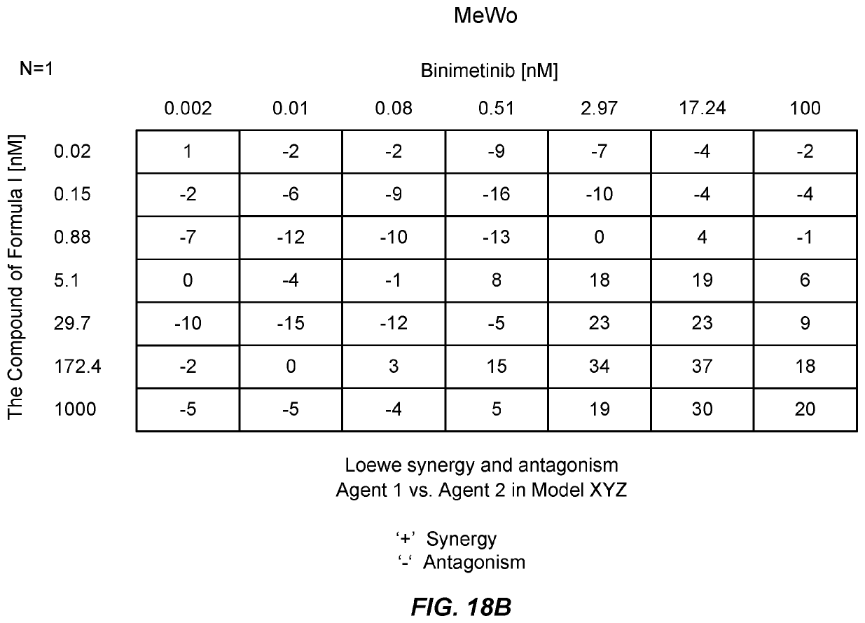

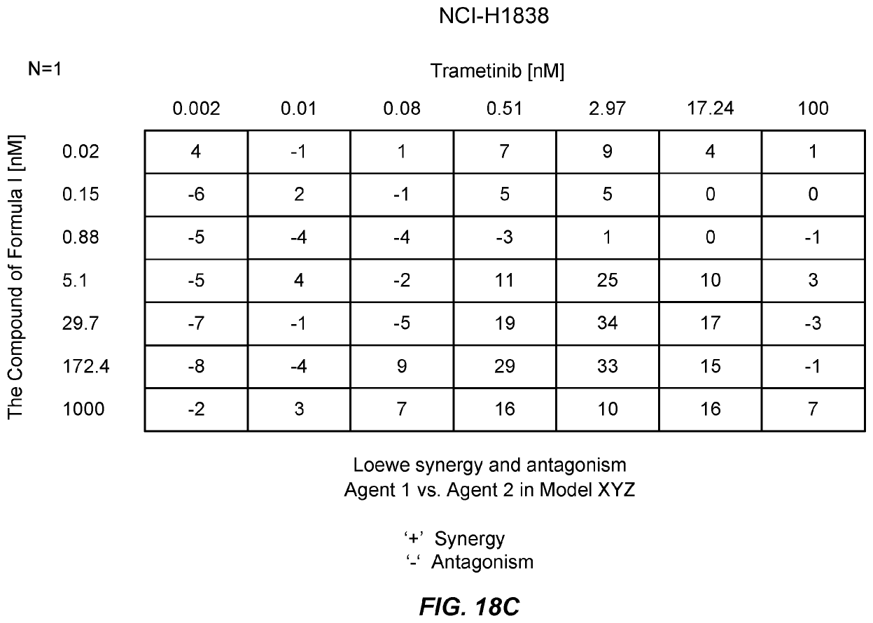

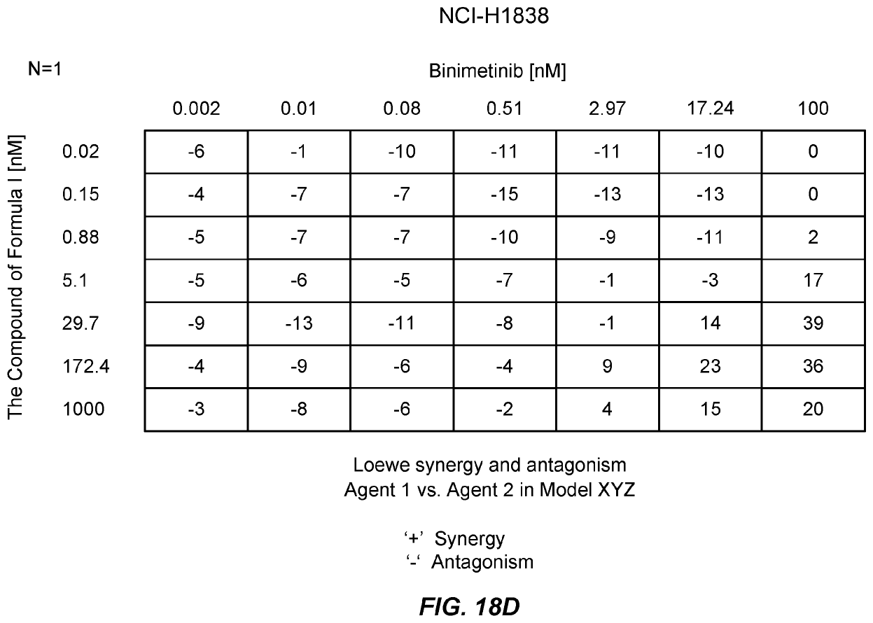

FIG. 18A shows synergy data in MeWo cancer cell line using the combination of the compound of Formula I and trametinib. FIG. 18B shows synergy data in MeWo cancer cell line using the combination of the compound of Formula I and binimetinib. FIG. 18C shows synergy data in NCI-H1838 cancer cell line using the combination of the compound of Formula I and trametinib. FIG. 18D shows synergy data in NCI-H1838 cancer cell line using the combination of the compound of Formula I and binimetinib.

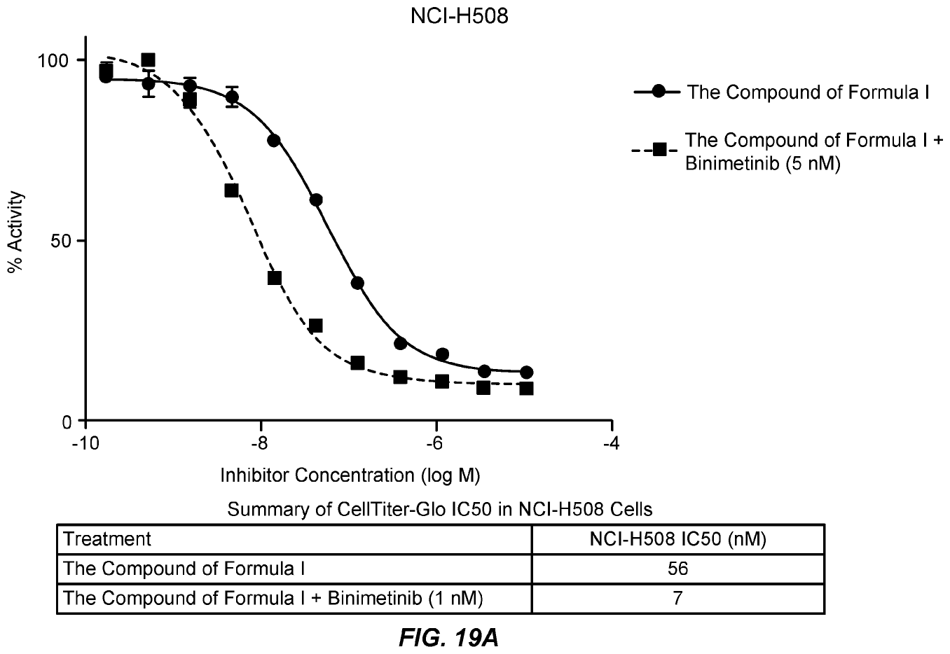

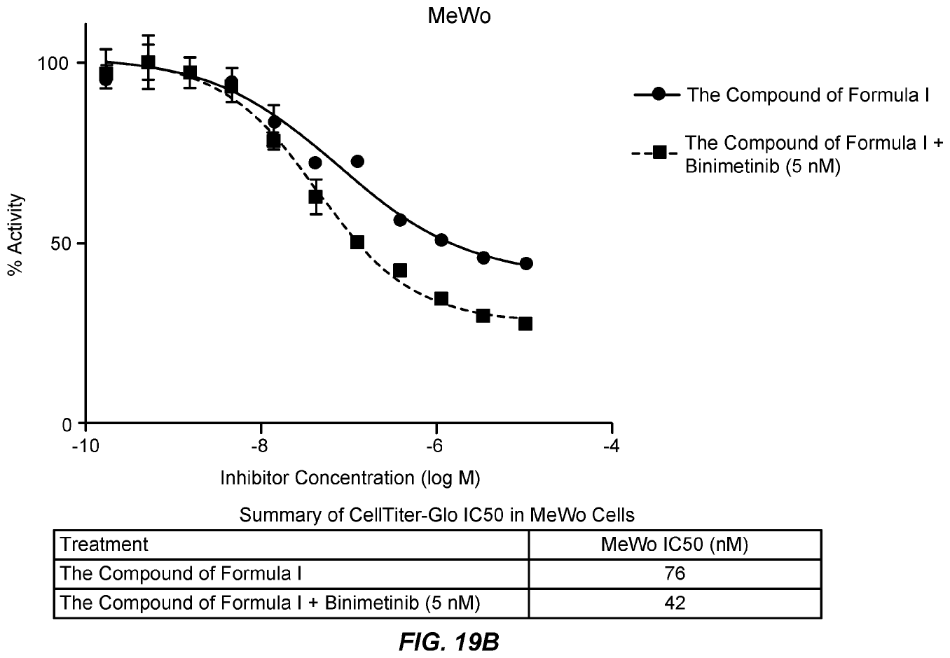

FIG. 19A shows a plot of percent activity versus inhibitor concentration (log M) in NCI-H508 cells treated with the compound of Formula I alone and in combination with binimetinib. The tabulated IC₅₀ data in NCI-H508 cells treated with the compound of Formula I alone and in combination with binimetinib. FIG. 19B shows a plot of percent activity versus inhibitor concentration (log M) in MeWo cells treated with the compound of Formula I alone and in combination with binimetinib. Tabulated IC50 data in MeWo cells treated with the compound of Formula I alone and in combination with binimetinib.

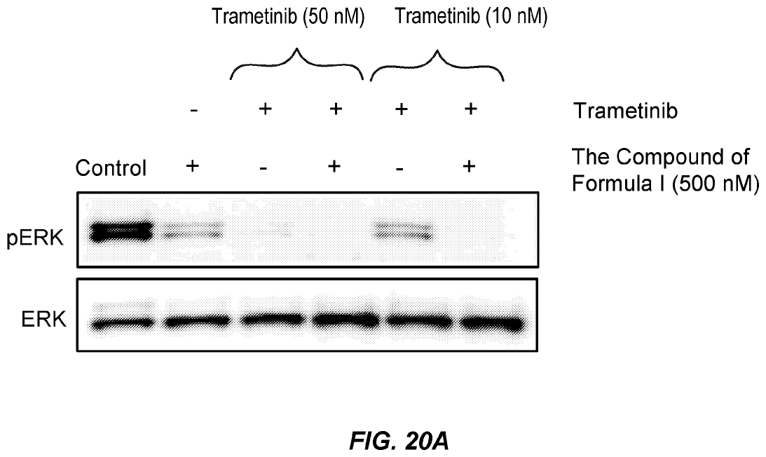

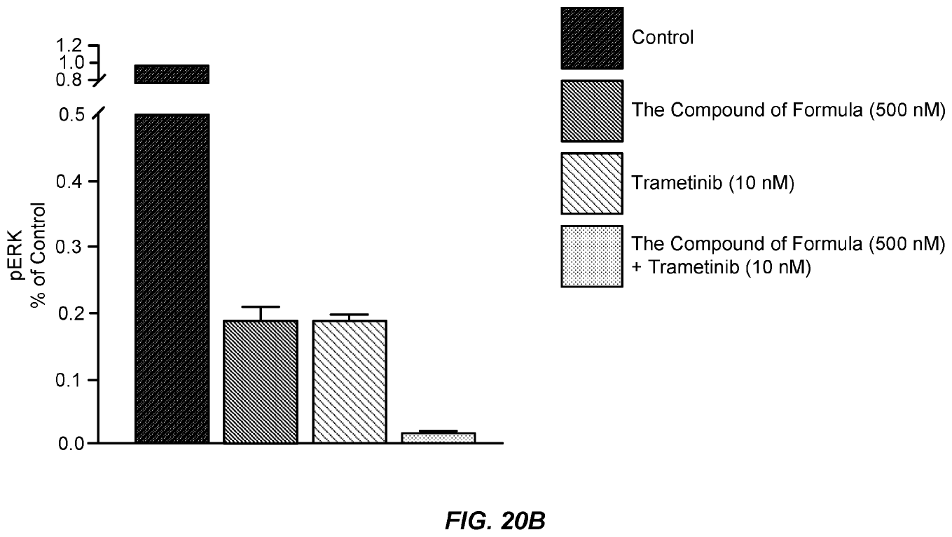

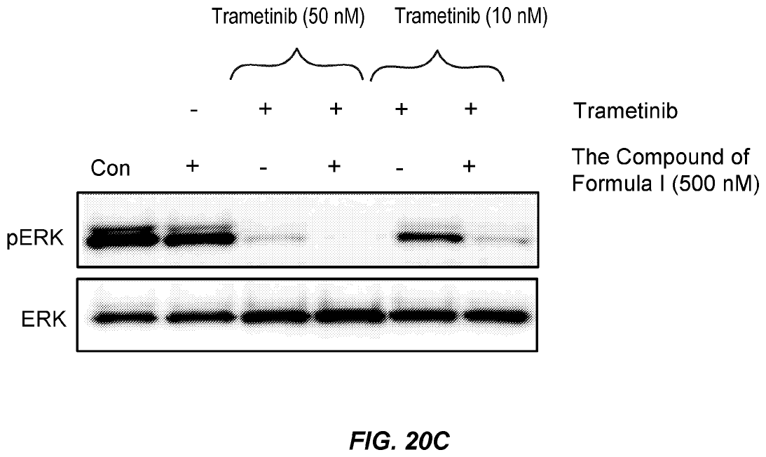

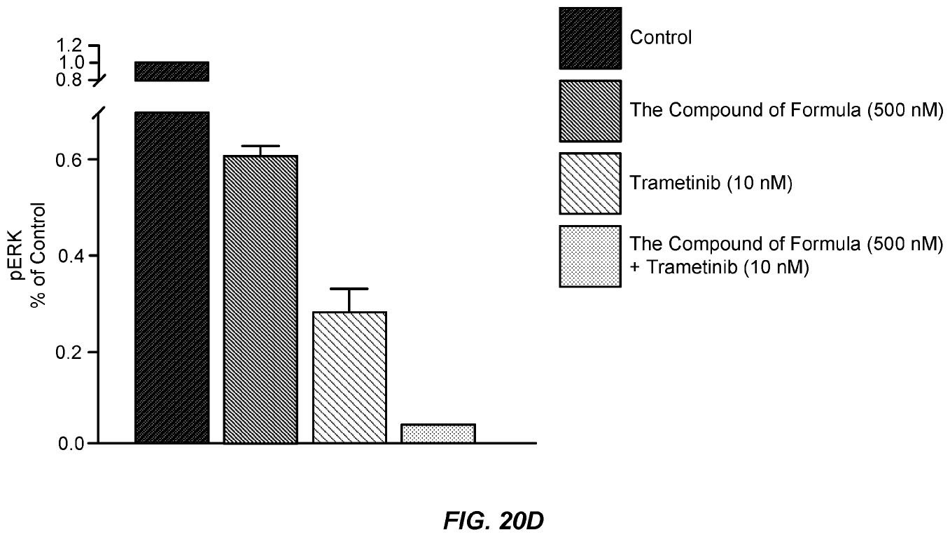

FIG. 20A shows a Western blot gel indicating the synergistic inhibition of ERK1/2 phosphorylation in the NCI-H508 cancer cell line. FIG. 20B shows a bar graph quantitation of the Western blot of FIG. 20A. FIG. 20C shows a Western blot gel indicating the synergistic inhibition of ERK1/2 phosphorylation in the MeWo (NF1 LoF) cancer cell line. FIG. 20D shows a bar graph quantitation of the Western blot of FIG. 20C.

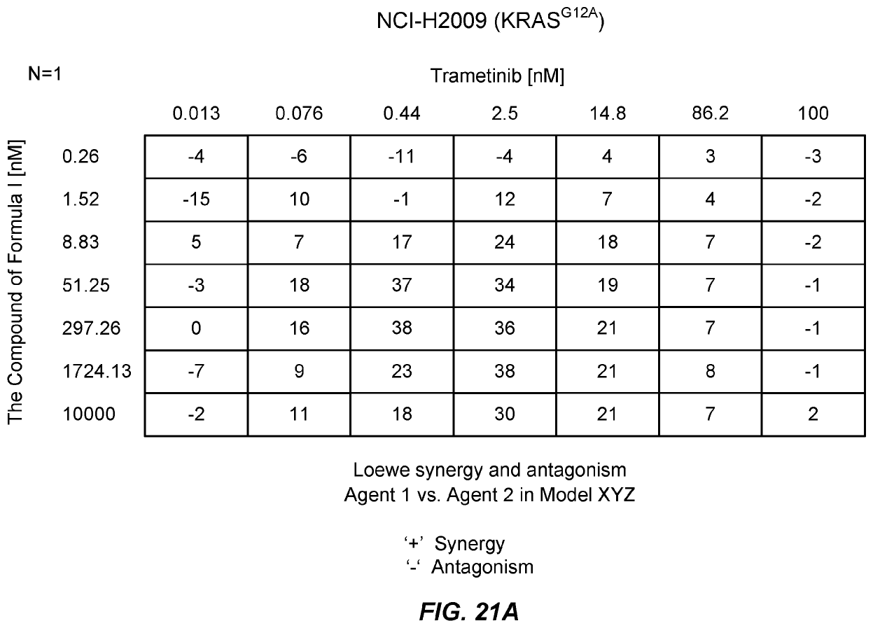

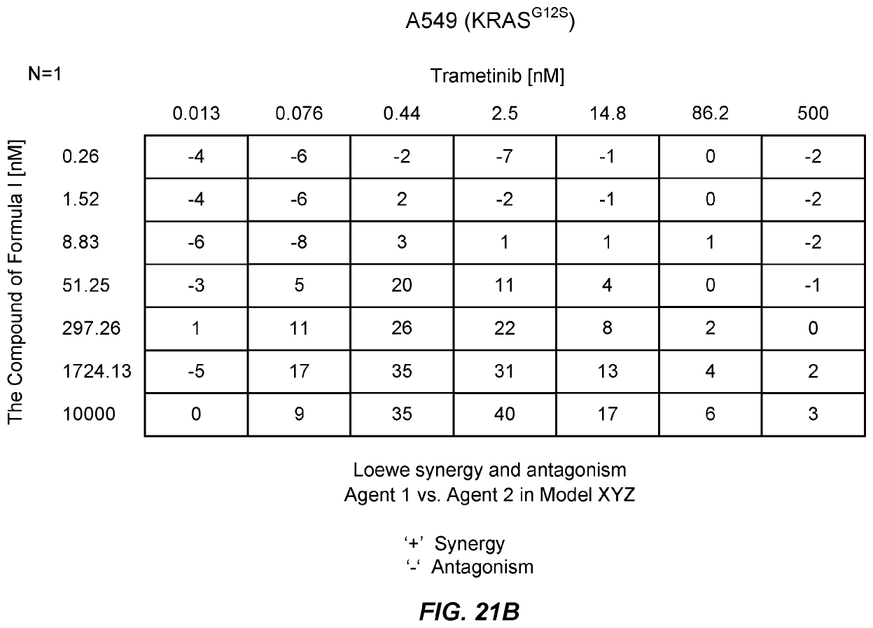

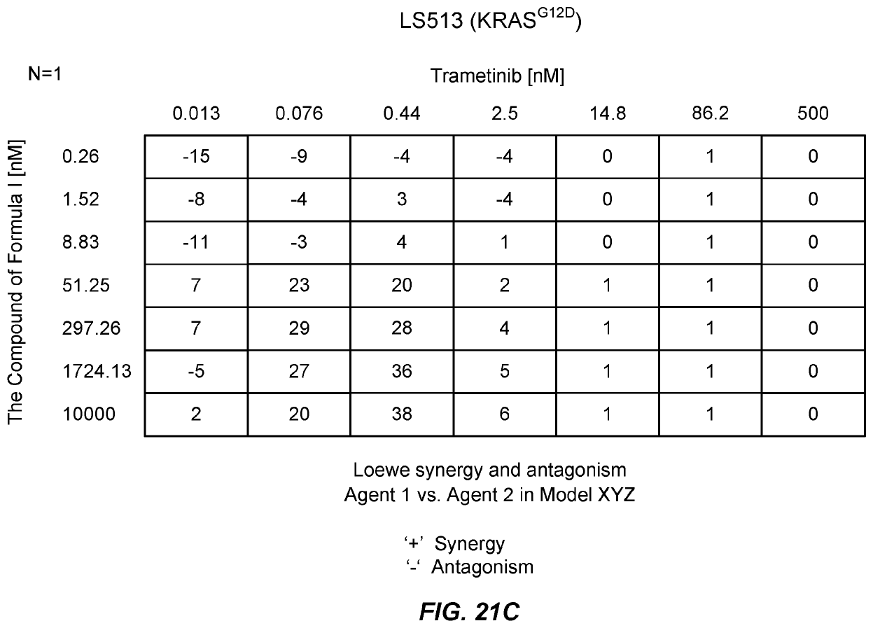

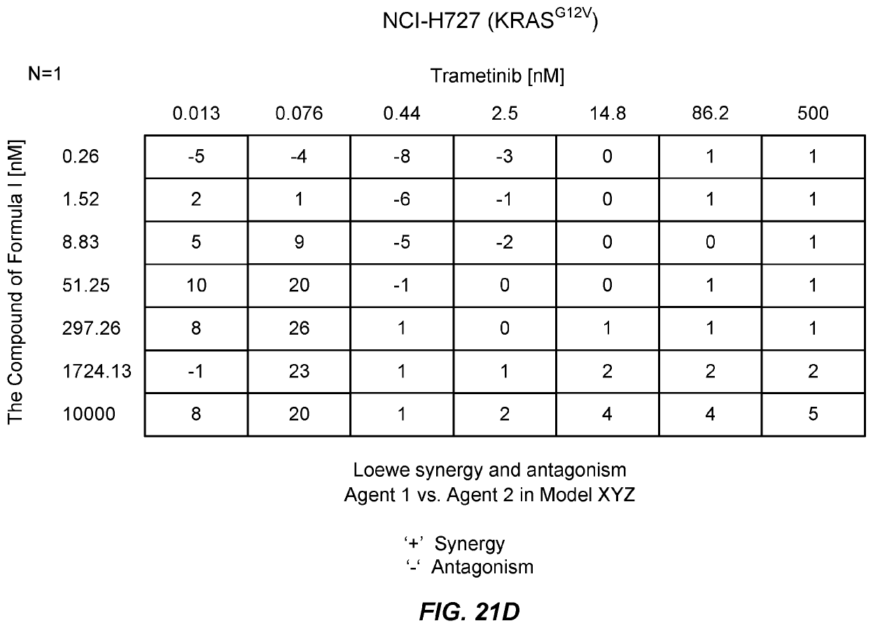

FIG. 21A shows synergy data in NCI-H2009 (KRAS G12A) cancer cell line using the combination of the compound of Formula I and trametinib. FIG. 21B shows synergy data in LS513 (KRAS G12D) cancer cell line using the combination of the compound of Formula I and trametinib. FIG. 21C shows synergy data in A549 (KRAS G12S) cancer cell line using the combination of the compound of Formula I and trametinib. FIG. 21D shows synergy data in NCI-H727 (KRAS G12V) cancer cell line using the combination of the compound of Formula I and trametinib.

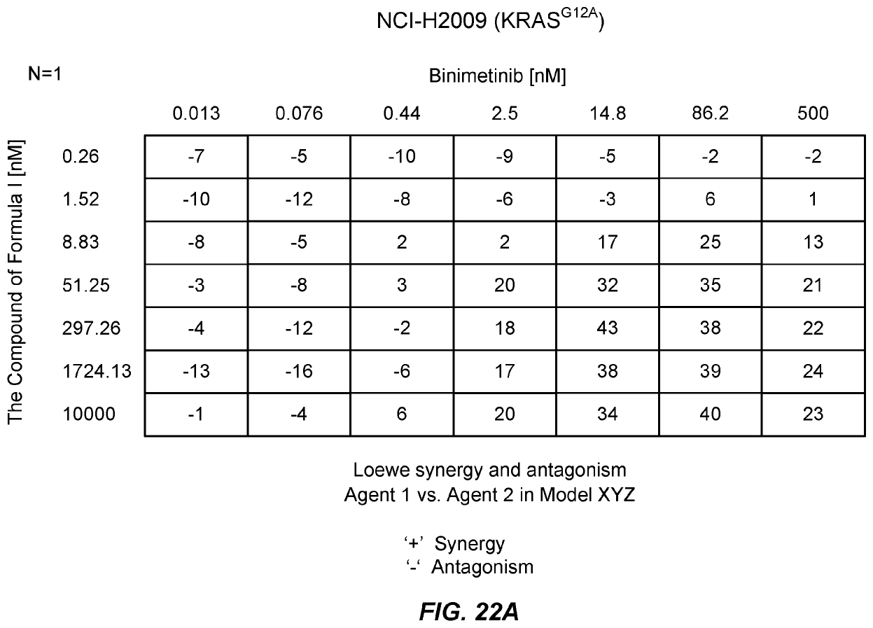

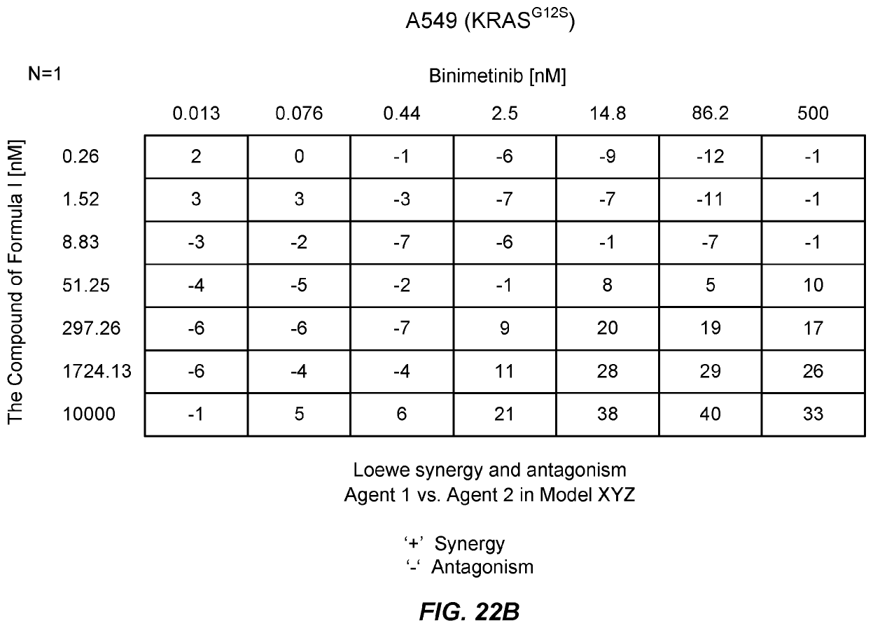

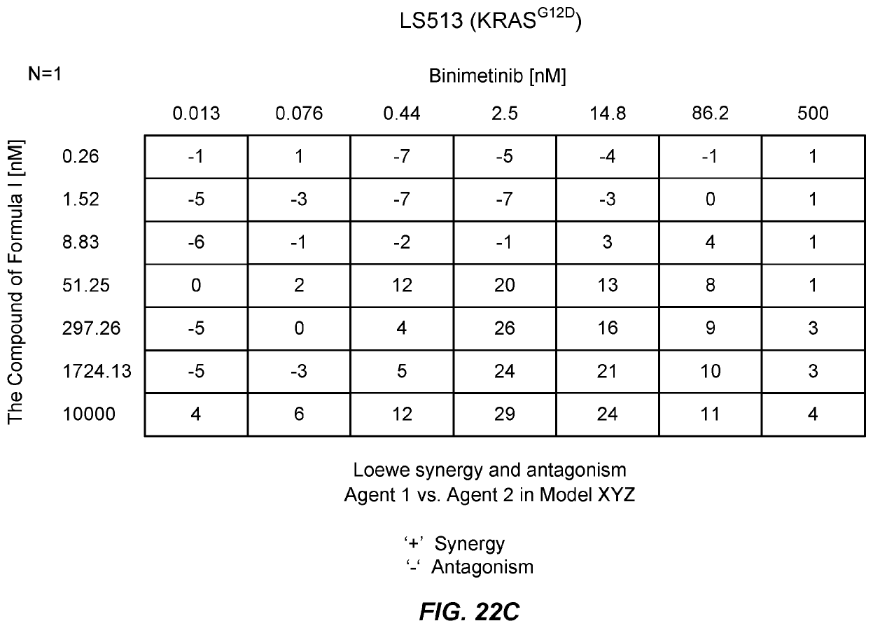

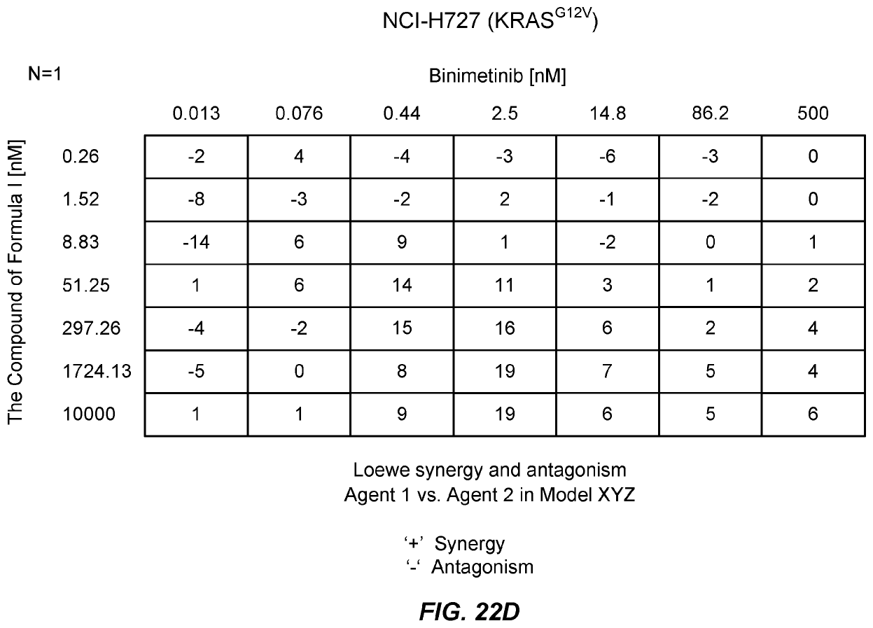

FIG. 22A shows synergy data in NCI-H2009 (KRAS G12A) cancer cell line using the combination of the compound of Formula I and binimetinib. FIG. 22B shows synergy data in LS513 (KRAS G12D) cancer cell line using the combination of the compound of Formula I and binimetinib. FIG. 22C shows synergy data in A549 (KRAS G12S) cancer cell line using the combination of the compound of Formula I and binimetinib. FIG. 22D shows synergy data in NCI-H727 (KRAS G12V) cancer cell line using the combination of the compound of Formula I and binimetinib.

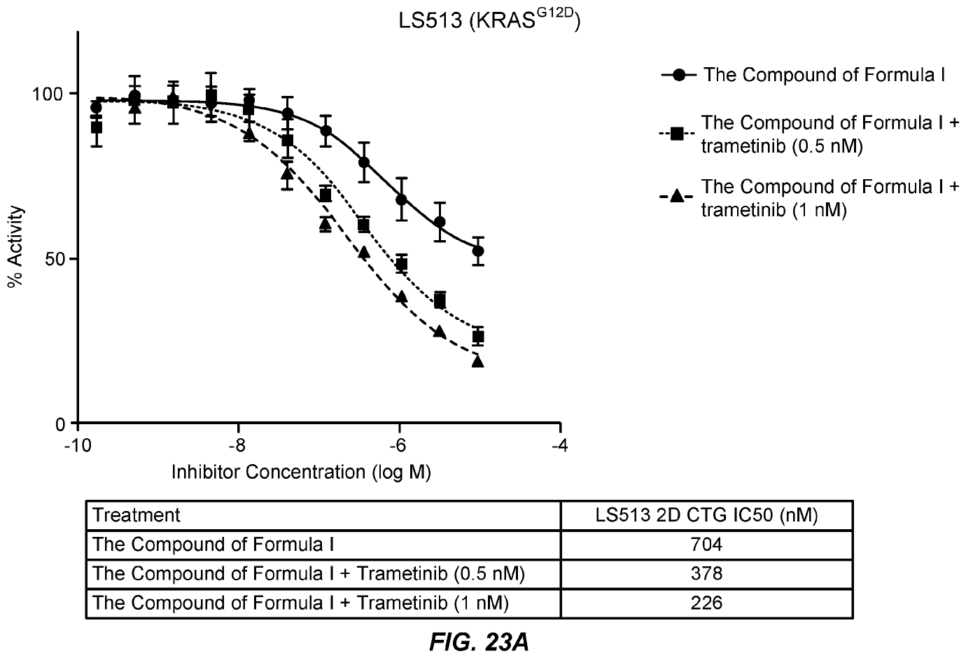

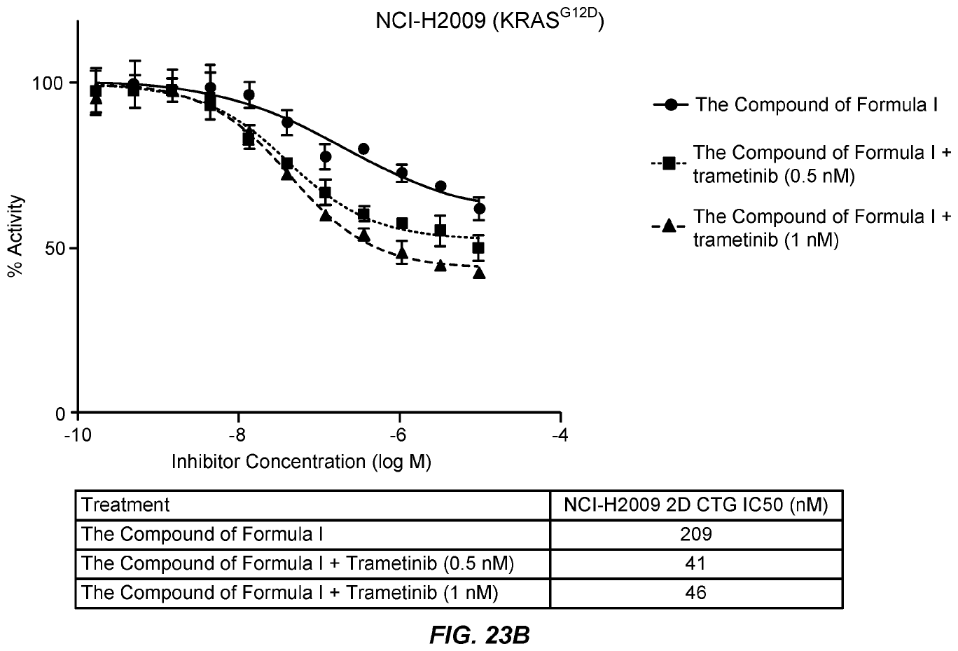

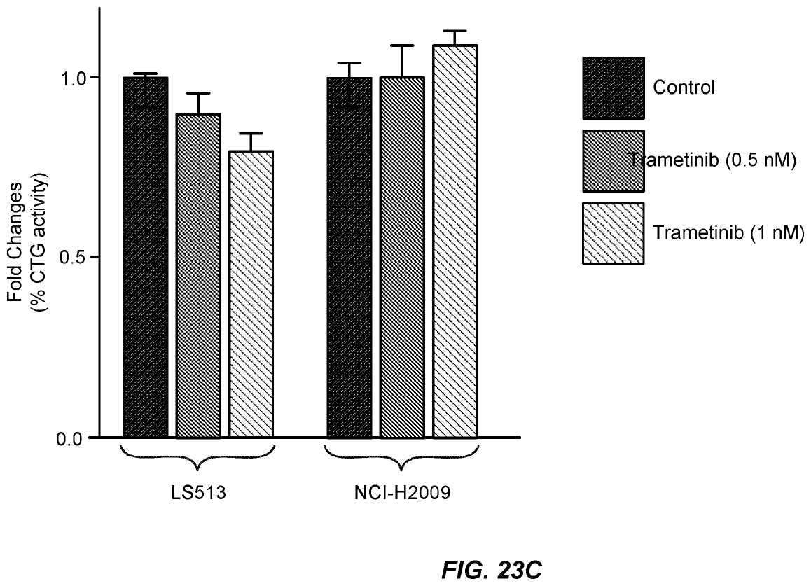

FIG. 23A shows a plot of percent activity versus inhibitor concentration (log M) in LS513 (KRAS G12D) cells treated with the compound of Formula I alone and in combination with trametinib. FIG. 23B shows a plot of percent activity versus inhibitor concentration (log M) in NCI-H2009 (KRAS G12D) cells treated with the compound of Formula I alone and in combination with trametinib. The tabulated data in NCI-H508 cells treated with the compound of Formula I alone and in combination with trametinib. FIG. 23C shows a bar graph of percent CTG activity that indicates Formula I or trametinib alone has minimal effect on cell viability. Collectively, this data indicates that combination of the compound of Formula I and inhibitors of MEK provides synergistic inhibition of cancer cell viability in BRAF class III, NF1 LoF and KRAS G12X mutated cancer.

### Example 13—Combination Therapy of the Compound of Formula I and Trametinib in NF1 LoF Mutant Melanoma CDX Model MeWo

### Materials

The vehicle/control article, 100 mM acetic acid in deionized water, with pH adjustment to 4.8-5.0, was prepared and stored under ambient conditions throughout the 28-day administration in mice.

The test article of the compound of Formula I was freshly prepared in vehicle of 100 mM acetic buffer weekly and stored under ambient conditions. The combination agent trametinib was freshly prepared in vehicle of 0.5% IPMC and 0.2% Tween 80 weekly and stored under ambient conditions.

Female Balb/c nude mice were purchased from the Beijing Vital River Laboratory Animal Technology Co., Ltd. Mice were between 6-8 weeks of age at the time of implantation. Mice were hosted at special pathogen-free (SPF) environment of vivarium facility and acclimated to their new environment for at least 3 days prior to initiation of any experiments according to IACUC protocol.

All procedures related to animal handling, care, and treatment in this study were performed according to guidelines approved by the Institutional Animal Care and Use Committee (IACUC) of WuXi AppTec. During the study, the care and use of animals were conducted in accordance with the regulations of the Association for Assessment and Accreditation of Laboratory Animal Care (AAALAC). In addition, all portions of this study performed at WuXi AppTec adhered to the study protocols approved by the study director and applicable standard operating procedures (SOPs).

### Preparation of Xenograft Model

MeWo was a human melanoma cell line that harbored a NF1 Q1336* mutation. The MeWo cell line was purchased from the American Type Culture Collection (ATCC® HTB-65™). MeWo cells were cultured in medium containing Minimum Essential Media (MEM) plus 10% Fetal Bovine Serum (FBS), 1% non-essential amino acid (NEAA), and 1% Antibiotic-Antimycotic (AA) at 37° C. in an atmosphere of 5% CO₂ in air. The medium was renewed every 2 to 3 days and tumor cells were routinely sub-cultured at a confluence of 80-90% by trypsin-EDTA. The cells growing in an exponential growth phase were harvested and counted for inoculation.

MeWo tumor cells were implanted into mice subcutaneously. 200 μL cell suspensions containing 5×106 tumor cells mixed with 50% Matrigel were subcutaneously implanted into the right flank of mouse using a syringe. Animal health and tumor growth were monitored daily. Tumor volume was measured twice a week by caliper when tumors were palpable and measurable. When tumor volumes reached a mean of 191 mm3 (range of 150-242 mm3), tumor-bearing mice were randomized into different groups with 8 mice in each group. The randomization date was denoted as treatment day 0.

### Treatment

Treatment started on the day after randomization. The treatment start day was denoted as treatment day 1. Mice were dosed by oral administration of vehicle control, the compound of Formula I alone at 10 mg/kg/dose BID, the compound of Formula I alone at 30 mg/kg QD, and trametinib alone at 0.4 mg/kg QD. Two groups received combination treatment of the compound of Formula I and trametinib, with one group dosed with the compound of Formula I at 10 mg/kg/dose BID and the other group dosed with the compound of Formula I at 30 mg/kg QD. Both combination groups were dosed with trametinib at 0.4 mg/kg QD. The dosing volume was 5 mL/kg and interval of BID regimen was 8 hours. Trametinib was dosed one hour after the first dose of the compound of Formula I BID or QD schedule in the combination groups.

### Results

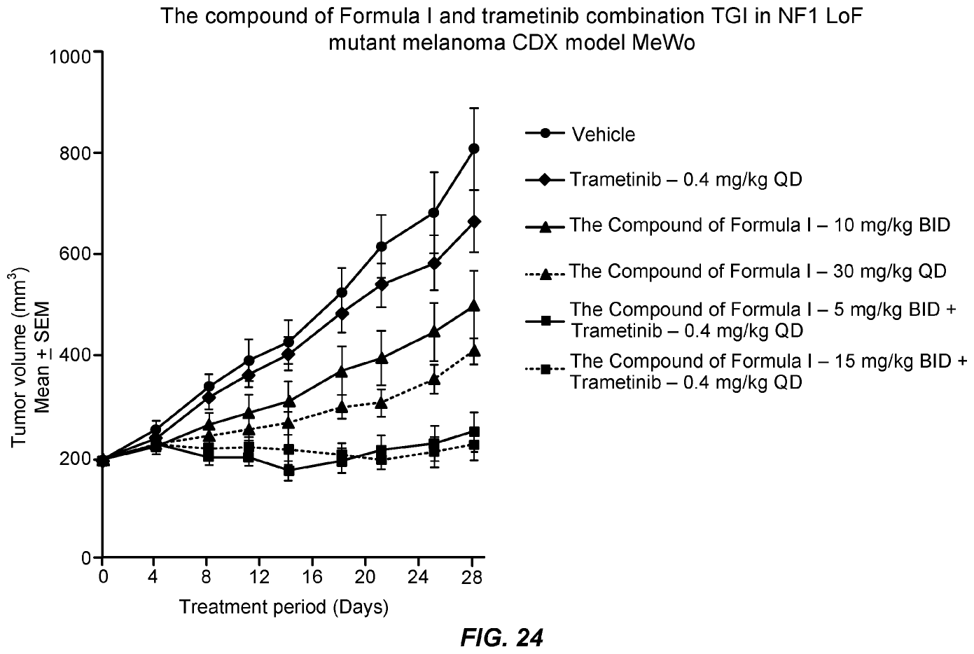

FIG. 24 shows a graph of tumor volume over a period of treatment time with the compound of Formula I alone, trametinib alone, and the combination of the compound of Formula I and trametinib in NF1 LoF Mutant Melanoma CDX Model MeWo. No significant body weight change was observed in the control and treatment groups.

### Conclusion

As shown in FIG. 24, the combination of the compound of Formula I and trametinib demonstrated superior tumor growth inhibition relative to treatment with the compound of Formula I alone or treatment with trametinib alone in NF1 LoF Mutant Melanoma CDX Model MeWo.

### Example 14—Combination Therapy of the Compound of Formula I and Binimetinib in NF1 LoF Mutant Melanoma CDX Model MeWo

### Materials

The vehicle/control article, 100 mM acetic acid in deionized water, with pH adjustment to 4.8-5.0, was prepared and stored under ambient conditions throughout the 28-day administration in mice.

The test article of the compound of Formula I was freshly prepared in vehicle of 100 mM acetic buffer weekly and stored under ambient conditions. The combination agent binimetinib was freshly prepared in vehicle of 1.0% MC and 0.5% Tween 80 weekly and stored at 2-8° C.

Female Balb/c nude mice were purchased from the Beijing Vital River Laboratory Animal Technology Co., Ltd. Mice were between 6-8 weeks of age at the time of implantation. Mice were hosted at special pathogen-free (SPF) environment of vivarium facility and acclimated to their new environment for at least 3 days prior to initiation of any experiments according to IACUC protocol.

All procedures related to animal handling, care, and treatment in this study were performed according to guidelines approved by the Institutional Animal Care and Use Committee (IACUC) of GenenDesign. During the study, the care and use of animals were conducted in accordance with the regulations of the Association for Assessment and Accreditation of Laboratory Animal Care (AAALAC). In addition, all portions of this study performed at GenenDesign adhered to the study protocols approved by the study director and applicable standard operating procedures (SOPs).

### Preparation of Xenograft Model

MeWo was a human melanoma cell line that harbored a NF1 Q1336* mutation. The MeWo cell line was purchased from the American Type Culture Collection (ATCC® HTB-65™). MeWo cells were cultured in medium containing Minimum Essential Media (MEM) plus 10% Fetal Bovine Serum (FBS), 1% non-essential amino acid (NEAA), and 1% Antibiotic-Antimycotic (AA) at 37° C. in an atmosphere of 5% C02 in air. The medium was renewed every 2 to 3 days and tumor cells were routinely sub-cultured at a confluence of 80-90% by trypsin-EDTA. The cells growing in an exponential growth phase were harvested and counted for inoculation.

MeWo tumor cells were implanted into mice subcutaneously. Briefly, 200 μL cell suspensions containing 5×106 tumor cells mixed with 50% Matrigel were subcutaneously implanted into the right flank of mouse using a syringe. Animal health and tumor growth were monitored daily. Tumor volume was measured twice a week by caliper when tumors were palpable and measurable. When tumor volumes reached a mean of 195 mm3 (range of 141-267 mm3), tumor-bearing mice were randomized into different groups with 8 mice in each group. The randomization date was denoted as treatment day 0.

### Treatment

Treatment started on the day after randomization. The treatment start day was denoted as treatment day 1. Mice were dosed by oral administration of vehicle control solution, the compound of Formula I alone at 15 mg/kg QD, the compound of Formula I alone at 30 mg/kg QD, binimetinib alone at 6 mg/kg BID, and binimetinib alone at 9 mg/kg/dose BID. Two additional groups received combination treatment of the compound of Formula I and binimetinib, with one group dosed with the compound of Formula I at 15 mg/kg QD and binimetinib at 6 mg/kg BID, and the other group dosed with the compound of Formula I at 15 mg/kg QD and binimetinib at 9 mg/kg/dose BID. The dosing volume was 5 mL/kg and interval of BID regimen was 8 hours. Binimetinib was dosed one hour after the compound of Formula I QD dose in the combination groups. The study was terminated on treatment day 28 as defined in the study protocol.

### Results

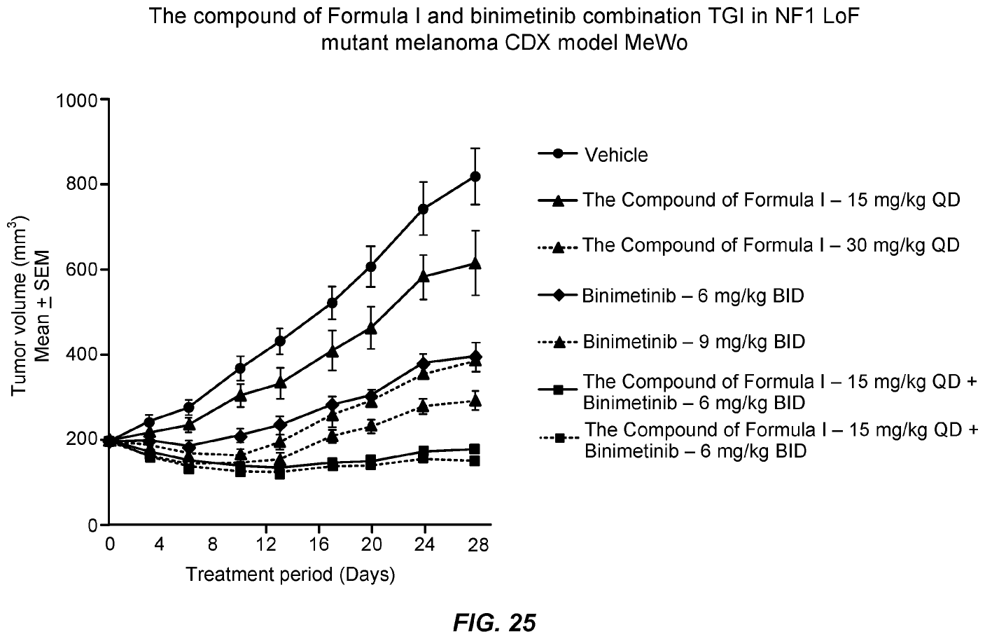

FIG. 25 shows a graph of tumor volume over a period of treatment time with the compound of Formula I alone, binimetinib alone, and the combination of the compound of Formula I and binimetinib in NF1 LoF Mutant Melanoma CDX Model MeWo. No significant body weight change was observed in the control and treatment groups.

### Conclusion

As shown in FIG. 25, the combination of the compound of Formula I and binimetinib demonstrated superior tumor growth inhibition relative to treatment with the compound of Formula I alone or treatment with binimetinib alone in NF1 LoF Mutant Melanoma CDX Model MeWo.

### Example 15—Combination Therapy of the Compound of Formula I and Trametinib in BRAF Class III Mutant CRC CDX Model NCI-11508

### Materials

The vehicle/control article, 100 mM acetic acid in deionized water, with pH adjustment to 4.8-5.0, was prepared and stored under ambient conditions throughout the 28-day administration in mice.

The test article of the compound of Formula I was freshly prepared in vehicle of 100 mM acetic buffer weekly and stored under ambient conditions. The combination agent trametinib was freshly prepared in vehicle of 0.5% IPMC and 0.2% Tween 80 weekly and stored under ambient conditions.

Female Balb/c nude mice were purchased from the Beijing Vital River Laboratory Animal Technology Co., Ltd. Mice were between 6-8 weeks of age at the time of implantation. Mice were hosted at special pathogen-free (SPF) environment of vivarium facility and acclimated to their new environment for at least 3 days prior to initiation of any experiments according to IACUC protocol.

All procedures related to animal handling, care, and treatment in this study were performed according to guidelines approved by the Institutional Animal Care and Use Committee (IACUC) of WuXi AppTec. During the study, the care and use of animals were conducted in accordance with the regulations of the Association for Assessment and Accreditation of Laboratory Animal Care (AAALAC). In addition, all portions of this study performed at WuXi AppTec adhered to the study protocols approved by the study director and applicable standard operating procedures (SOPs).

### Preparation of Xenograft Model

NCI-H508 was a human CRC cell line that harbored a BRAF class III mutation (BRAF G596R). The NCI-H508 cell line was purchased from the American Type Culture Collection (ATCC® CCL-253™). NCI-H508 cells were cultured in medium containing RPMI-1640 plus 10% Fetal Bovine Serum (FBS) and 1% Antibiotic-Antimycotic (AA) at 37° C. in an atmosphere of 5% C02 in air. The medium was renewed every 2 to 3 days and tumor cells were routinely sub-cultured at a confluence of 80-90% by trypsin-EDTA. The cells growing in an exponential growth phase were harvested and counted for inoculation.

NCI-H508 tumor cells were implanted into mice subcutaneously. Briefly, 200 μL cell suspensions containing 10×106 tumor cells mixed with 50% Matrigel were subcutaneously implanted into the right flank of mouse using a syringe. Animal health and tumor growth were monitored daily. Tumor volume was measured twice a week by caliper when tumors were palpable and measurable. When tumor volumes reached a mean of 182 mm3 (range of 108-287 mm3), tumor-bearing mice were randomized into different groups with 8 mice in each group. The randomization date was denoted as treatment day 0.

### Treatment

Treatment started on the day after randomization. The treatment start day was denoted as treatment day 1. Mice were dosed by oral administration of vehicle control, the compound of Formula I alone at 10 mg/kg BID, the compound of Formula I alone at 30 mg/kg QD, and trametinib alone at 0.4 mg/kg QD. Two groups received combination treatment of the compound of Formula I and trametinib, with one group dosed with the compound of Formula I at 10 mg/kg BID and trametinib at 0.4 mg/kg QD, and the other group dosed with the compound of Formula I and trametinib at 0.4 mg/kg QD at 30 mg/kg QD. The dosing volume was 5 mL/kg and interval of BID regimen was 8 hours. Trametinib was dosed one hour after the first dose of the compound of Formula BID or QD dose in the combination groups.

### Results

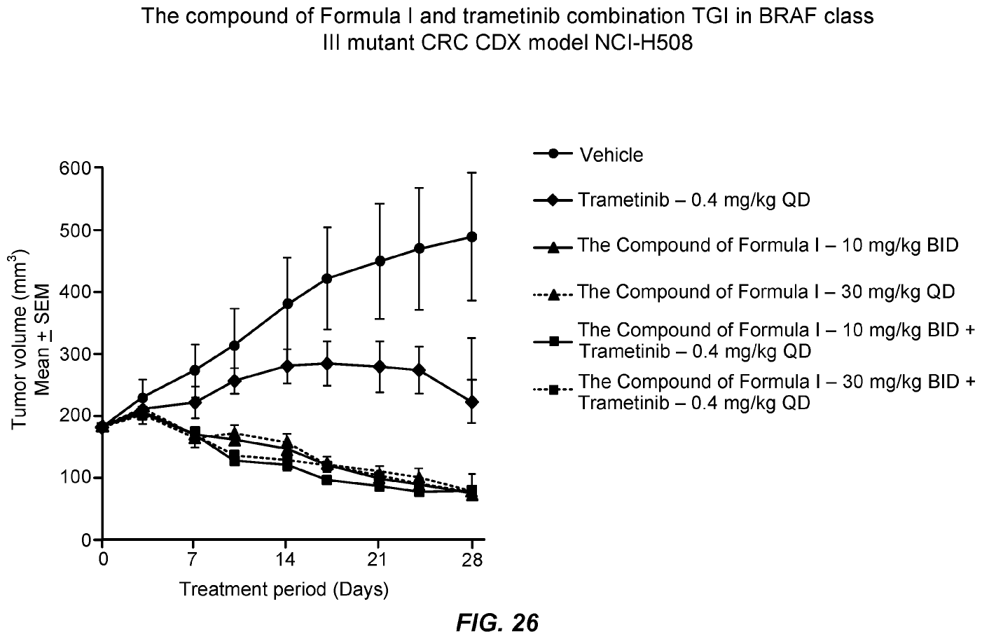

FIG. 26 shows a graph of tumor volume over a period of treatment time with the compound of Formula I alone, trametinib alone, and the combination of the compound of Formula I and trametinib in BRAF Class III Mutant CRC CDX Model NCI-H508. No significant body weight change was observed in the control and treatment groups.

### Conclusion

As shown in FIG. 26, the combination of the compound of Formula I and trametinib demonstrated superior tumor growth inhibition relative to treatment with the compound of Formula I alone or treatment with trametinib alone in BRAF Class III Mutant CRC CDX Model NCI-H508.

### Example 16—Combination Therapy of the Compound of Formula I and Trametinib in NF1 LoF Mutant NSCLC CDX Model NCI-111838

### Materials

The vehicle/control article, 100 mM acetic acid in deionized water, with pH adjustment to 4.8-5.0, was prepared and stored under ambient conditions throughout the 28-day administration in mice.

The test article of the compound of Formula I was freshly prepared in vehicle of 100 mM acetic buffer weekly and stored under ambient conditions. The combination agent trametinib was freshly prepared in vehicle of 0.5% HPMC and 0.2% Tween 80 weekly and stored under ambient conditions.

Female SCID Beige mice (Cat #405) were purchased from the Beijing Vital River Laboratory Animal Technology Co., Ltd. Mice were between 6-8 weeks of age at the time of implantation. Mice were hosted at special pathogen-free (SPF) environment of vivarium facility and acclimated to their new environment for at least 3 days prior to initiation of any experiments according to IACUC protocol.

All procedures related to animal handling, care, and treatment in this study were performed according to guidelines approved by the Institutional Animal Care and Use Committee (IACUC) of WuXi AppTec. During the study, the care and use of animals were conducted in accordance with the regulations of the Association for Assessment and Accreditation of Laboratory Animal Care (AAALAC). In addition, all portions of this study were performed at WuXi AppTec and adhered to the study protocols approved by the study director and applicable standard operating procedures (SOPs).

### Preparation of Xenograft Model

NCI-H1838 was a human lung adenocarcinoma cell line that harbored the NF1LOF mutation, NF1 N184fs. The NCI-H1838 cell line was purchased from the American Type Culture Collection (ATCC® CRL-5899™). NCI-H1838 cells were cultured in medium containing RPMI-1640 plus 10% Fetal Bovine Serum (FBS) and 1% Antibiotic-Antimycotic (AA) at 37° C. in an atmosphere of 5% C02 in air. The medium was renewed every 2 to 3 days and tumor cells were routinely sub-cultured at a confluence of 80-90% by trypsin-EDTA. The cells growing in an exponential growth phase were harvested and counted for inoculation.

NCI-H1838 tumor cells were implanted into mice subcutaneously. Briefly, 200 μL cell suspensions containing 10×106 tumor cells mixed with 50% Matrigel were subcutaneously implanted into the right flank of mouse using a syringe. Animal health and tumor growth were monitored daily. Tumor volume was measured twice a week by caliper when tumors were palpable and measurable. When tumor volumes reached a mean of 254 mm3 (range of 149-503 mm3), tumor-bearing mice were randomized into different groups with 8 mice in each group. The randomization date was denoted as treatment day 0.

### Treatment

Treatment started on the day after randomization. The treatment start day was denoted as treatment day 1. Mice were dosed by oral administration of vehicle control, the compound of Formula I alone at 10 mg/kg BID, the compound of Formula I alone at 30 mg/kg QD, and trametinib alone at 0.4 mg/kg QD. Two groups received the combination treatment of the compound of Formula I and trametinib, with one group dosed with the compound of Formula I at 10 mg/kg BID and trametinib at 0.4 mg/kg QD, and the other group dosed with the compound of Formula I at 30 mg/kg QD and trametinib at 0.4 mg/kg QD. The dosing volume was 5 mL/kg and interval of BID regimen was 8 hours. Trametinib was dosed one hour after the first dose of the compound of Formula I dose or QD dose in the combination groups.

### Results

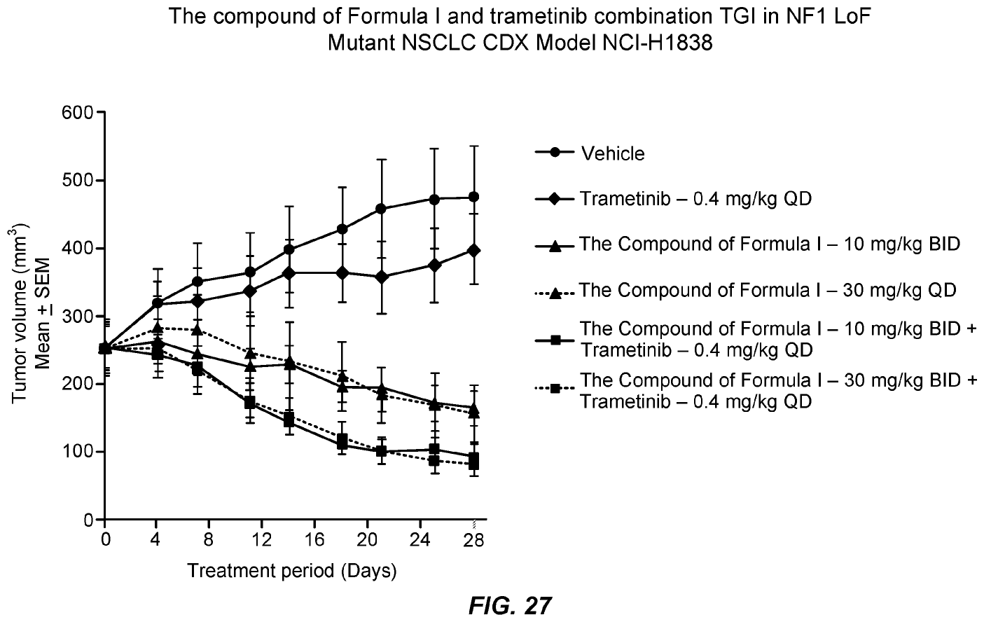

FIG. 27 shows a graph of tumor volume over a period of treatment time with the compound of Formula I alone, trametinib alone, and the combination of the compound of Formula I and trametinib in NF1 LoF Mutant NSCLC CDX Model NCI-H1838. No significant body weight change was observed in the control and treatment groups.

### Conclusion

As shown in FIG. 27, the combination of the compound of Formula I and trametinib demonstrated superior tumor growth inhibition relative to treatment with the compound of Formula I alone or treatment with trametinib alone in NF1 LoF Mutant NSCLC CDX Model NCI-H1838.

### Example 17—Synergistic Combination of the Compound of Formula I and Inhibitors of MET

### Combination Cellular Proliferation Assays

Cells (2000 cells per well) were plated onto 96-well plates in 100 μl cell culture medium. Cells were treated with the compound of Formula I and crizotinib at concentrations varying from 0 to 10 μM by using the Tecan D300e Digital Dispenser combination matrix protocol. At day 5, 50 μl of CellTiter-Glo (CTG) reagent (Promega) was added and the plates were incubated for 10 minutes with gentle shaking. After 10 minutes of incubation, the luminescent signal was determined according to the provider's instructions (Promega) and combination data was generated by the standard HSA model using Combenefit software. The combination synergy was represented by positive numbers in results table. The negative numbers represent antagonism of the combination.

### Results

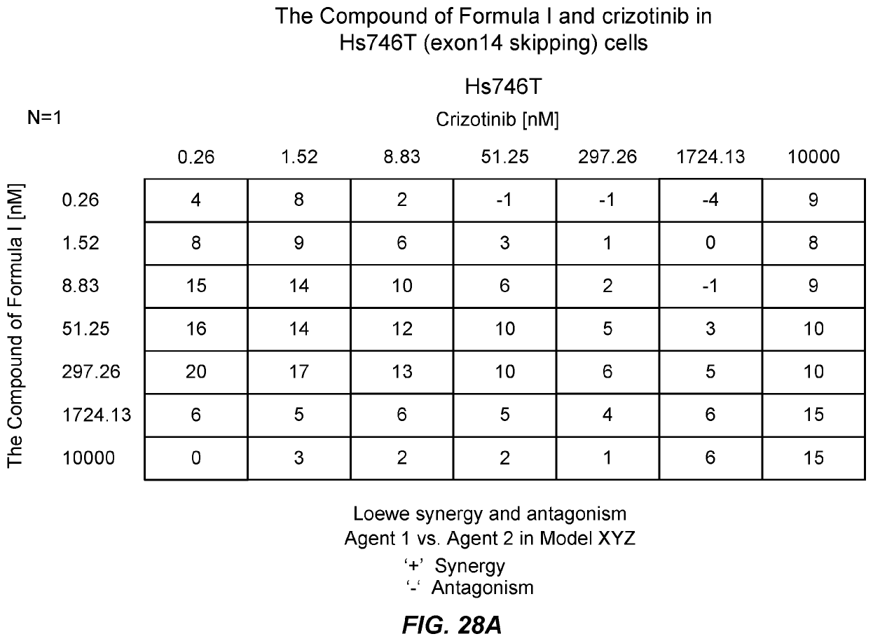

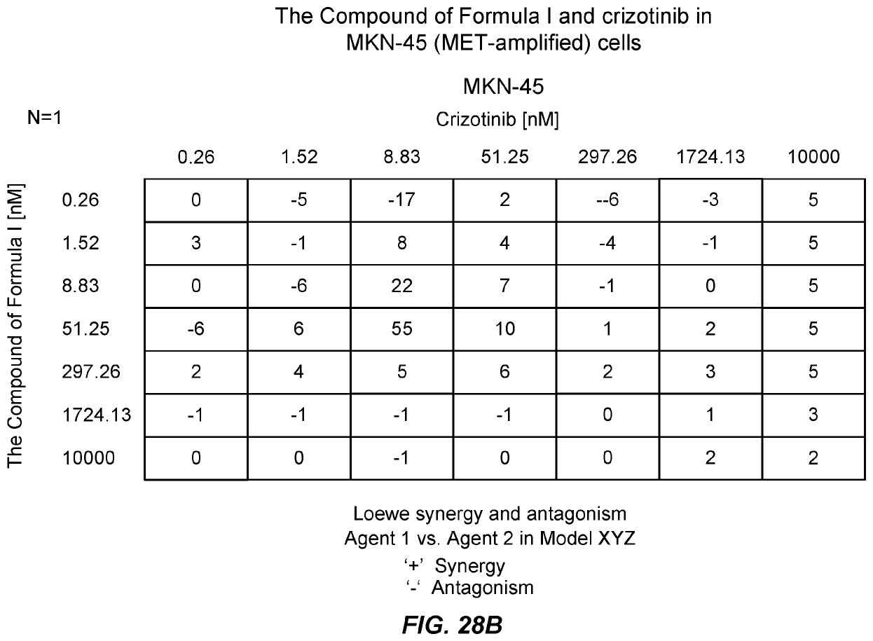

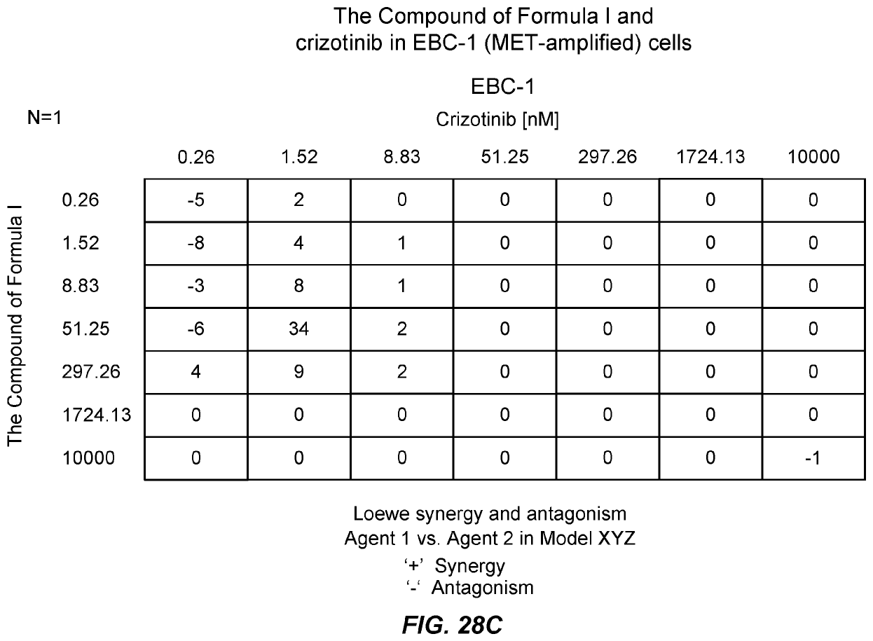

FIG. 28A shows synergy data in Hs746T cancer cell line using the combination of the compound of Formula I and crizotinib. FIG. 28B shows synergy data in MKN-45 cancer cell line using the combination of the compound of Formula I and crizotinib. FIG. 28C shows synergy data in EBC-1 cancer cell line using the combination of the compound of Formula I and crizotinib.

### Example 18—Combination Therapy of the Compound of Formula I and Crizotinib in c-MET Amplified Gastric Cancer CDX Model SNU-5

### Materials

The vehicle/control article, 100 mM acetic acid in deionized water, with pH adjustment to 4.8-5.0, was prepared and stored under ambient conditions throughout the 28-day administration in mice.

The test article of the compound of Formula I was freshly prepared in vehicle of 100 mM acetic buffer weekly and stored under ambient conditions. The combination agent crizotinib was prepared in vehicle of 0.5% Methyl Cellulose and stored under 2-8° C.

Female Balb/c nude mice were purchased from the Beijing Vital River Laboratory Animal Technology Co., Ltd. Mice were between 6-8 weeks of age at the time of implantation. Mice were hosted at special pathogen-free (SPF) environment of vivarium facility and acclimated to their new environment for at least 3 days prior to initiation of any experiments according to IACUC protocol. All procedures related to animal handling, care, and treatment in this study were performed according to guidelines approved by the Institutional Animal Care and Use Committee (IACUC) of WuXi AppTec. During the study, the care and use of animals were conducted in accordance with the regulations of the Association for Assessment and Accreditation of Laboratory Animal Care (AAALAC). In addition, all portions of this study performed at WuXi AppTec adhered to the study protocols approved by the study director and applicable standard operating procedures (SOPs).

### Preparation of Xenograft Model

SNU-5 was a c-MET amplified gastric cancer cell line. The SNU-5 cell line was purchased from the American Type Culture Collection (ATCC® CRL-5973™). SNU-5 cells were cultured in medium containing IMDM (Iscove's Modified Dulbecco's Medium) plus 20% Fetal Bovine Serum (FBS) and 1% Antibiotic-Antimycotic (AA), at 37° C. in an atmosphere of 5% C02 in air. The medium was renewed every 2 to 3 days and tumor cells were routinely sub-cultured at a confluence of 80-90% by trypsin-EDTA. Cells growing in an exponential growth phase were harvested and counted for inoculation. SNU-5 tumor cells (passage 13) were implanted into mice subcutaneously. 200 μL cell suspensions containing 10×106 tumor cells were subcutaneously implanted into the right flank of mouse using a syringe. Animal health and tumor growth were monitored daily. Tumor volume was measured twice a week by caliper when tumors were palpable and measurable. When tumor volumes reached a mean of 227 mm3 at day 34 post subcutaneous implantation, tumor-bearing mice were randomized into different groups with 8 mice in each group. The randomization date was denoted as treatment day 0.

### Treatment

Treatment started on the day after randomization. The treatment start day was denoted as treatment day 1. Mice were dosed by oral administration of vehicle control solution, the compound of Formula I alone at 10 mg/kg BID, the compound of Formula I alone at 30 mg/kg QD, and crizotinib alone at 50 mg/kg BID. Two additional groups received combination treatment of the compound of Formula I and crizotinib, with one group dosed with the combination of the compound of Formula I at 5 mg/kg BID and crizotinib at 50 mg/kg BID, and the other group dosed with the combination of the compound of Formula I at 15 mg/kg QD and crizotinib at 50 mg/kg BID. The dosing volume was 5 mL/kg and interval of BID regimen was 8 hours. Crizotinib was dosed one hour after the dosing of the compound of Formula I QD or the first dose of the BID dose schedule in the combination groups. The study was terminated on treatment day 28 as defined in the study protocol.

### Results

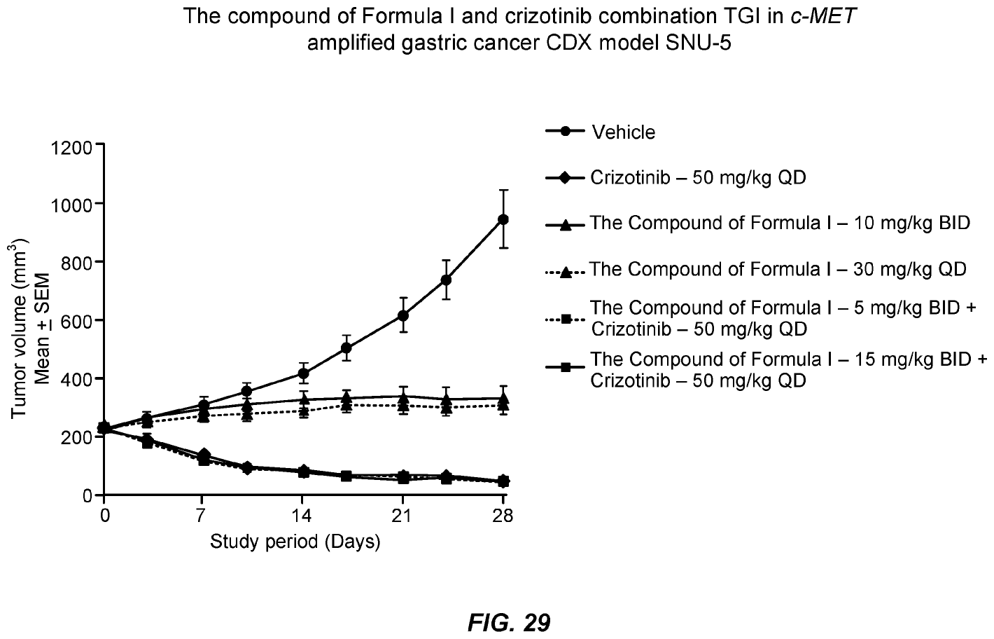

FIG. 29 shows a graph of tumor volume over a period of treatment time with the compound of Formula I alone, crizotinib alone, and the combination of the compound of Formula I and crizotinib in c-MET amplified gastric cancer CDX model SNU-5. No significant body weight change was observed in the control and treatment groups.

### Conclusion

As shown in FIG. 29, the combination of the compound of Formula I and crizotinib demonstrated superior tumor growth inhibition relative to treatment with the compound of Formula I alone or treatment with crizotinib alone in c-MET amplified gastric cancer CDX model SNU-5.

### Example 19—Combination Therapy of the Compound of Formula I and Crizotinib in c-MET Amplified NSCLC CDX Model NCI-H1993

### Materials

The vehicle/control article, 100 mM acetic acid in deionized water, with pH adjustment to 4.8-5.0, was prepared and stored under ambient conditions throughout the 28-day administration in mice.

The test article of the compound of Formula I was freshly prepared in vehicle of 100 mM acetic buffer weekly and stored under ambient conditions. The combination agent crizotinib was prepared in vehicle of 0.5% Methyl Cellulose and stored under 2-8° C.

Female Balb/c nude mice were purchased from the Beijing Vital River Laboratory Animal Technology Co., Ltd. Mice were between 6-8 weeks of age at the time of implantation. Mice were hosted at special pathogen-free (SPF) environment of vivarium facility and acclimated to their new environment for at least 3 days prior to initiation of any experiments according to IACUC protocol. All procedures related to animal handling, care, and treatment in this study were performed according to guidelines approved by the Institutional Animal Care and Use Committee (IACUC) of WuXi AppTec. During the study, the care and use of animals were conducted in accordance with the regulations of the Association for Assessment and Accreditation of Laboratory Animal Care (AAALAC). In addition, all portions of this study performed at WuXi AppTec adhered to the study protocols approved by the study director and applicable standard operating procedures (SOPs).

### Preparation of Xenograft Model

NCI-H1993 was a c-MET amplified NSCLC cell line. The NCI-H1993 cell line was purchased from the American Type Culture Collection (ATCC® CRL-5909™). NCI-H1993 cells were cultured in medium containing RPMI-1640 plus 10% Fetal Bovine Serum (FBS) and 1% Antibiotic-Antimycotic (AA), at 37° C. in an atmosphere of 5% C02 in air. The medium was renewed every 2 to 3 days and tumor cells were routinely sub-cultured at a confluence of 80-90% by trypsin-EDTA. Cells growing in an exponential growth phase were harvested and counted for inoculation. NCI-H1993 tumor cells (passage 13) were implanted into mice subcutaneously. 200 μL cell suspensions containing 5×106 tumor cells mixed with 50% Matrigel were subcutaneously implanted into the right flank of mouse using a syringe. Animal health and tumor growth were monitored daily. Tumor volume was measured twice a week by caliper when tumors were palpable and measurable. When tumor volumes reached a mean of 201 mm3 at day 10 post subcutaneous implantation, tumor-bearing mice were randomized into different groups with 8 mice in each group. The randomization date was denoted as treatment day 0.

### Treatment

Treatment started on the day after randomization. The treatment start day was denoted as treatment day 1. Mice were dosed by oral administration of vehicle control solution, the compound of Formula I alone at 10 mg/kg BID, the compound of Formula I alone at 30 mg/kg QD, and crizotinib alone at 50 mg/kg BID. One additional group received combination treatment of the compound of Formula I at 5 mg/kg BID and crizotinib at 50 mg/kg BID. The dosing volume was 5 mL/kg and interval of BID regimen was 8 hours. Crizotinib was dosed one hour after the first dose of the compound of Formula I BID dose in the combination group. The study was terminated on treatment day 28 as defined in the study protocol.

### Results

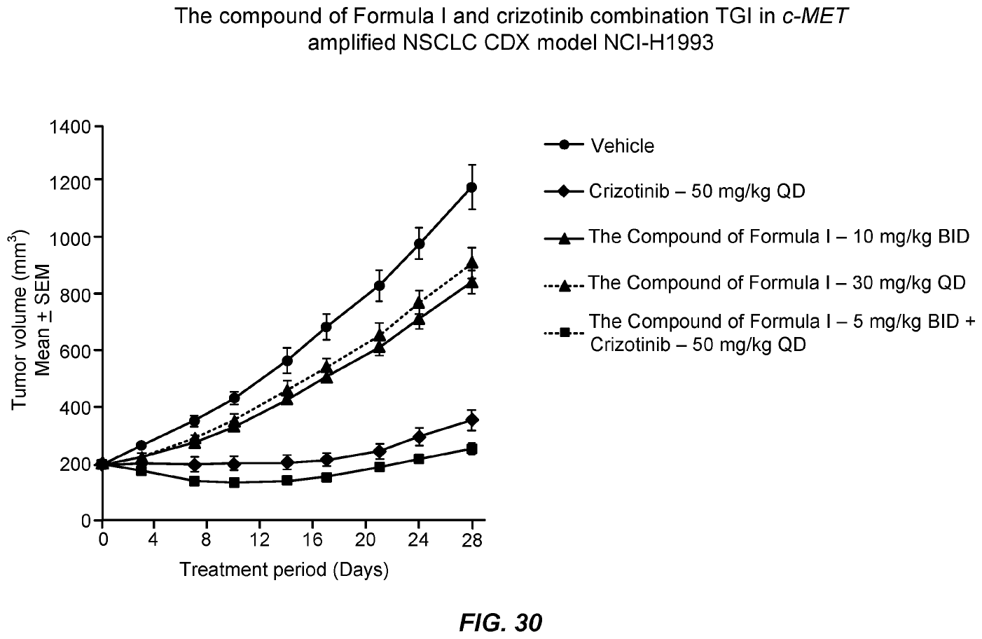

FIG. 30 shows a graph of tumor volume over a period of treatment time with the compound of Formula I alone, crizotinib alone, and the combination of the compound of Formula I and crizotinib in c-MET amplified NSCLC CDX model NCI-H1993. No significant body weight change was observed in the control and treatment groups.

### Conclusion

As shown in FIG. 30, the combination of the compound of Formula I and crizotinib demonstrated superior tumor growth inhibition relative to treatment with the compound of Formula I alone or treatment with crizotinib alone in c-MET amplified NSCLC CDX model NCI-H1993.

Although the foregoing embodiments have been described in some detail by way of illustration and Examples for purposes of clarity of understanding, one of skill in the art will appreciate that certain changes and modifications may be practiced within the scope of the appended claims. In addition, each reference provided herein is incorporated by reference in its entirety to the same extent as if each reference was individually incorporated by reference. Where a conflict exists between the instant application and a reference provided herein, the instant application shall dominate.

1. A method of treating a subject having cancer comprising administering to the subject a therapeutically effective amount of a compound of Formula I or its pharmaceutically acceptable salt: 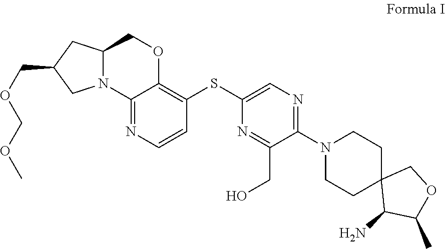 [SMILES: COCOC[C@H]1C[C@H]2COc3c(Sc4cnc(N5CCC6(CC5)CO[C@@H](C)[C@H]6N)c(CO)n4)ccnc3N2C1]
in combination with an FGFR inhibitor.

2. The method of claim 1, wherein the FGFR in the subject is constitutively active.

3. The method of claim 1 or 2, wherein the cancer lung cancer.

4. The method of claim 1 or 2, wherein the cancer is hepatocellular carcinoma.

5. The method of claim 1 or 2, wherein the cancer is cholangiocarcinoma.

6. The method of claim 1 or 2, wherein the cancer is pancreatic ductal adenocarcinoma (PDAC).

7. The method of any one of claims 1 to 5, wherein the inhibitor is selected from the group consisting of erdafitinib, AZD4547, Ly2874455, CH5183284, NVP-BGJ398, INCB054828, rogaratinib, PRN1371, TAS-120, BLU-554, H3B-6527, andFGF401.

8. The method of any one of claims 1 to 5, wherein the FGFR inhibitor is erdafitinib.

9. The method of any one of claims 1 to 5, wherein the FGFR inhibitor is pemigatinib, infigratinib, dovitinib, ponatinib, nintedanib, and fisogatinib.

10. The method of any one of claims 1 to 9, wherein the method comprises administering a third MAPK pathway inhibitor.

11. The method of any one of claims 1 to 10, wherein the administration is oral.

12. The method of any one of claims 1 to 11, wherein the dosing of the compound of Formula I is in a range from 20 mg to 400 mg daily.

13. The method of any one of claims 1 to 12, wherein the dosing of the FGFR inhibitor is in a range from 1 mg to 500 mg daily.

14. A method of treating liver cancer in a subject comprising orally administering to the subject a therapeutically effective amount of a compound of Formula I or its pharmaceutically acceptable salt: 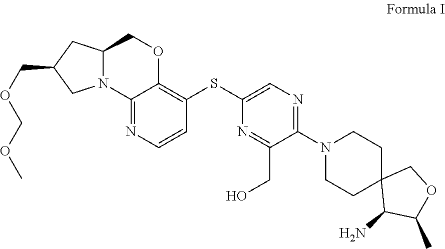 [SMILES: COCOC[C@H]1C[C@H]2COc3c(Sc4cnc(N5CCC6(CC5)CO[C@@H](C)[C@H]6N)c(CO)n4)ccnc3N2C1]
in combination with erdafitinib.

15. The method of claim 14, wherein the compound of Formula I is administered once or twice daily.

16. The method of claim 14 or 15, wherein erdafitinib is administered once or twice daily.

17. The method of claim 14, wherein the subject is a human.

18. A kit comprising a compound of Formula I or a pharmaceutically acceptable salt thereof and an FGFR inhibitor.

19. The kit of claim 18, wherein the compound of Formula I and the FGFR inhibitor are in separate packages.

20. The kit of claim 18 or 19, wherein the kit further comprises instructions to administer the contents of the kit to a subject for the treatment of cancer.

21. The kit of any of claims 18 to 20, wherein the FGFR inhibitor is one or more of erdafitinib, AZD4547, Ly2874455, CH5183284, NVP-BGJ398, INCB054828, rogaratinib, PRN1371, TAS-120, BLU-554, H3B-6527, FGF401, pemigatinib, infigratinib, dovitinib, ponatinib, nintedanib, and fisogatinib.

22. A method of treating a subject having cancer comprising administering to the subject a therapeutically effective amount of a compound of Formula I or its pharmaceutically acceptable salt: 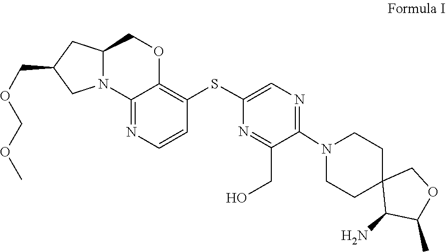 [SMILES: COCOC[C@H]1C[C@H]2COc3c(Sc4cnc(N5CCC6(CC5)CO[C@@H](C)[C@H]6N)c(CO)n4)ccnc3N2C1]
in combination with an inhibitor of a B-Raf protein having a class 1 mutation.

23. The method of claim 22, wherein the class 1 mutation is V600E.

24. The method of claim 22 or 23, wherein the cancer is colorectal cancer.

25. The method of claim 22 or 23, wherein the cancer is melanoma.

26. The method of claim 22 or 23, wherein the cancer is thyroid cancer.

27. The method of claim 22 or 23, wherein the cancer is pancreatic ductal adenocarcinoma (PDAC).

28. The method of any one of claims 22 to 27, wherein the inhibitor is selected from the group consisting of encorafenib, vemurafenib, dabrafenib, sorafenib, and regorafenib.

29. The method of any one of claims 22 to 27, wherein the inhibitor is encorafenib.

30. The method of any one of claims 22 to 27, wherein the inhibitor is vemurafenib.

31. The method of any one of claims 22 to 27, wherein the inhibitor is dabrafenib.

32. The method of any one of claims 22 to 27, wherein the inhibitor is sorafenib.

33. The method of any one of claims 22 to 27, wherein the inhibitor is regorafenib.

34. The method of any one of claims 22 to 33, wherein the method comprises administering a third MAPK pathway inhibitor.

35. The method of any one of claims 22 to 34, wherein the administration is oral.

36. The method of any one of claims 22 to 35, wherein the dosing of the compound of Formula I is in a range from 20 mg to 400 mg daily.

37. The method of any one of claims 22 to 36, wherein the dosing of the B-Raf inhibitor is in a range from 1 mg to 500 mg.

38. A method of treating colorectal cancer in a subject comprising orally administering to the subject a therapeutically effective amount of a compound of Formula I or its pharmaceutically acceptable salt: 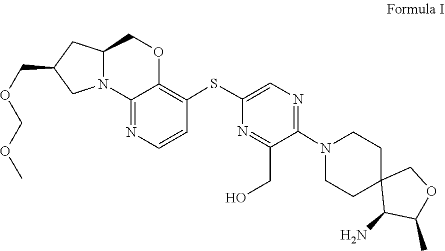 [SMILES: COCOC[C@H]1C[C@H]2COc3c(Sc4cnc(N5CCC6(CC5)CO[C@@H](C)[C@H]6N)c(CO)n4)ccnc3N2C1]
in combination with B-Raf inhibitor encorafenib.

39. The method of claim 38, wherein the compound of Formula I is administered once or twice daily.

40. The method of claim 38 or 39, wherein encorafenib is administered once or twice daily.

41. The method of claim any one of claims 38 to 40, wherein the subject is human.

42. A method of treating a subject having cancer comprising administering to the subject a therapeutically effective amount of a compound of Formula I or its pharmaceutically acceptable salt: 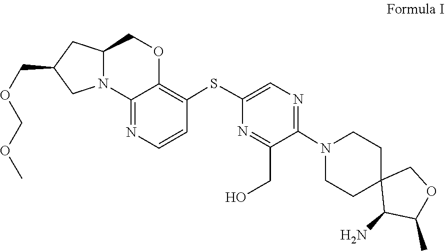 [SMILES: COCOC[C@H]1C[C@H]2COc3c(Sc4cnc(N5CCC6(CC5)CO[C@@H](C)[C@H]6N)c(CO)n4)ccnc3N2C1]
in combination with encorafenib.

43. A method of treating a subject having cancer comprising administering to the subject a therapeutically effective amount of a compound of Formula I or its pharmaceutically acceptable salt: 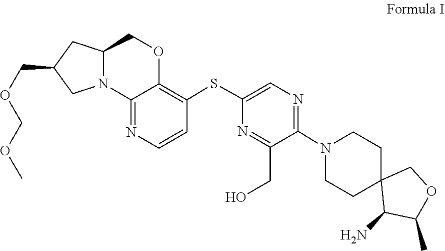 [SMILES: COCOC[C@H]1C[C@H]2COc3c(Sc4cnc(N5CCC6(CC5)CO[C@@H](C)[C@H]6N)c(CO)n4)ccnc3N2C1]
in combination with vemurafenib

44. A method of treating a subject having cancer comprising administering to the subject a therapeutically effective amount of a compound of Formula I or its pharmaceutically acceptable salt: 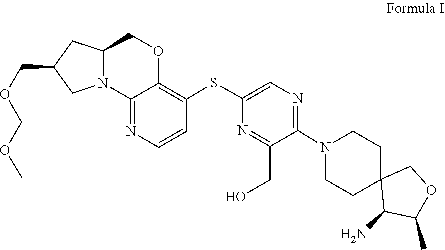 [SMILES: COCOC[C@H]1C[C@H]2COc3c(Sc4cnc(N5CCC6(CC5)CO[C@@H](C)[C@H]6N)c(CO)n4)ccnc3N2C1]
in combination with dabrafenib

45. A method of treating a subject having cancer comprising administering to the subject a therapeutically effective amount of a compound of Formula I or its pharmaceutically acceptable salt: 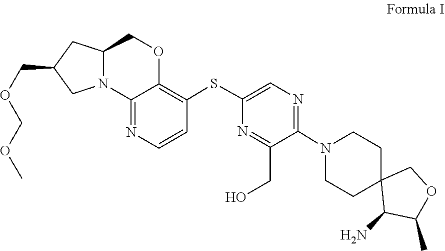 [SMILES: COCOC[C@H]1C[C@H]2COc3c(Sc4cnc(N5CCC6(CC5)CO[C@@H](C)[C@H]6N)c(CO)n4)ccnc3N2C1]
in combination with sorafenib.

46. A method of treating a subject having cancer comprising administering to the subject a therapeutically effective amount of a compound of Formula I or its pharmaceutically acceptable salt: 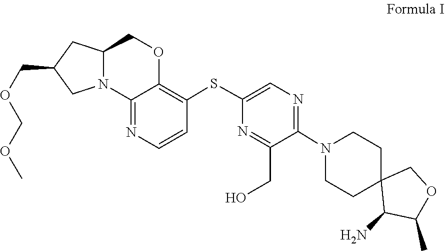 [SMILES: COCOC[C@H]1C[C@H]2COc3c(Sc4cnc(N5CCC6(CC5)CO[C@@H](C)[C@H]6N)c(CO)n4)ccnc3N2C1]
in combination with regorafenib.

47. The method of any one of claims 42 to 46, wherein the cancer is colorectal cancer.

48. The method of any one of claims 42 to 46, wherein the cancer is thyroid cancer.

49. The method of any one of claims 42 to 46, wherein the cancer is melanoma.

50. The method of any one of claims 42 to 46, wherein the cancer is pancreatic ductal adenocarcinoma (PDAC).

51. The method of any one of claims 22 to 50, wherein a dosing of the B-Raf inhibitor is less than a dosing required for a monotherapy with the B-Raf inhibitor.

52. The method of any one of claims 22 to 51, wherein a dosing of the compound of Formula I is less than a dosing required for a monotherapy with the compound of Formula I.

53. A method of inhibiting ERK1/2 phosphorylation in a cell population comprising contacting a cell population with the compound of Formula I or its pharmaceutically acceptable salt: 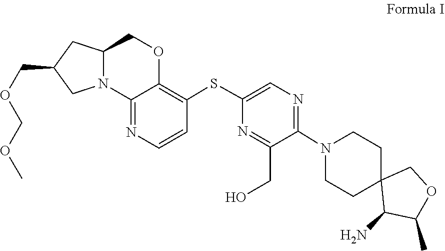 [SMILES: COCOC[C@H]1C[C@H]2COc3c(Sc4cnc(N5CCC6(CC5)CO[C@@H](C)[C@H]6N)c(CO)n4)ccnc3N2C1]
in combination with regorafenib.

54. The method of claim 53, wherein a concentration of the compound of Formula I is a range from 1 nM to 500 nM.

55. The method of claim 53 or 54, wherein a concentration of encorafenib is in a range from 10 nM to 20 nM.

56. A kit comprising a compound of Formula I or a pharmaceutically acceptable salt thereof and a B-Raf inhibitor.

57. The kit of claim 56, wherein the compound of Formula I and the B-Raf inhibitors are in separate packages.

58. The kid of claim 56 or 57, wherein the kit further comprises instructions to administer the contents of the kit to a subject for the treatment of cancer.

59. The kit of any one of claims 56 to 58, wherein the B-Raf inhibitor is one or more of encorafenib, vemurafenib, dabrafenib, sorafenib, and regorafenib.

60. A method of treating a subject having cancer comprising administering to the subject a therapeutically effective amount of a compound of Formula I or its pharmaceutically acceptable salt: 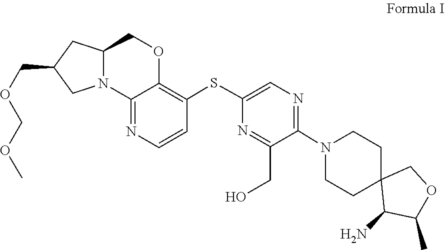 [SMILES: COCOC[C@H]1C[C@H]2COc3c(Sc4cnc(N5CCC6(CC5)CO[C@@H](C)[C@H]6N)c(CO)n4)ccnc3N2C1]
in combination with a MEK inhibitor.

61. The method of claim 60, wherein the MEK inhibitor inhibits MEK1 selectively or MEK2 selectively or both MEK1 and MEK2 selectively.

62. The method of claim 60, wherein the cancer is metastatic.

63. The method of any one of claims 60 to 62, wherein the cancer colorectal cancer.

64. The method of any one of claims 60 to 62, wherein the cancer is melanoma.

65. The method of any one of claims 60 to 62, wherein the cancer is lung cancer.

66. The method of any one of claims 60 to 62, wherein the cancer is pancreatic cancer.

67. The method of any one of claims 60 to 62, wherein the cancer is breast cancer.

68. The method of any one of claims 60 to 62, wherein the cancer is pancreatic ductal adenocarcinoma (PDAC).

69. The method of any one of claims 60 to 68, wherein the MEK inhibitor is selected from the group consisting of trametinib, cobimetinib, binimetinib, PD-0325901, selumetinib and CI-1040.

70. The method of any one of claims 60 to 68, wherein the MEK inhibitor is trametinib.

71. The method of any one of claims 60 to 68, wherein the MEK inhibitor is cobimetinib.

72. The method of any one of claims 60 to 68, wherein the MEK inhibitor is binimetinib.

73. The method of any one of claims 60 to 68, wherein the MEK inhibitor is PD-325901.

74. The method of any one of claims 60 to 68, wherein the MEK inhibitor is CI-1040.

75. The method of any one of claims 60 to 74, wherein the method comprises administering a further MAPK pathway inhibitor.

76. The method of any one of claims 60 to 75, wherein the administration is oral.

77. The method of any one of claims 60 to 76, wherein the dosing of the compound of Formula I is in a range from 20 mg to 400 mg daily.

78. The method of any one of claims 60 to 77, wherein the dosing of the MEK inhibitor is in a range from 1 mg to 500 mg daily.

79. A method of treating cancer in a subject comprising orally administering to the subject a therapeutically effective amount of a compound of Formula I or its pharmaceutically acceptable salt: 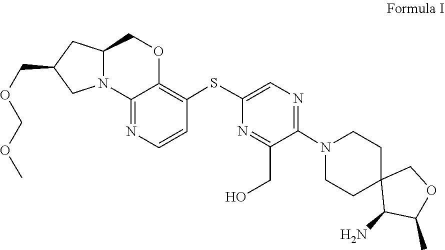 [SMILES: COCOC[C@H]1C[C@H]2COc3c(Sc4cnc(N5CCC6(CC5)CO[C@@H](C)[C@H]6N)c(CO)n4)ccnc3N2C1]
in combination with MEK inhibitor binimetinib or trametinib.

80. The method of claim 79, wherein the compound of Formula I is administered once or twice daily.

81. The method of claim 79 or 80, wherein binimetinib or trametinib is administered once or twice daily.

82. The method of any one of claims 79 to 81, wherein the subject is a human.

83. A method of treating a subject having cancer comprising administering to the subject a therapeutically effective amount of a compound of Formula I or its pharmaceutically acceptable salt: 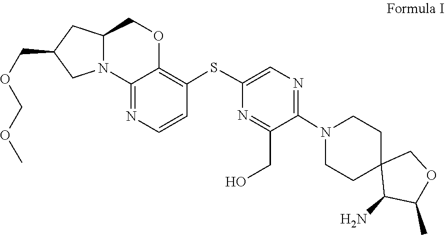 [SMILES: COCOC[C@H]1C[C@H]2COc3c(Sc4cnc(N5CCC6(CC5)CO[C@@H](C)[C@H]6N)c(CO)n4)ccnc3N2C1]
in combination with binimetinib.

84. A method of treating a subject having cancer comprising administering to the subject a therapeutically effective amount of a compound of Formula I or its pharmaceutically acceptable salt: 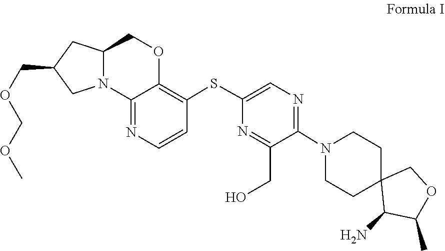 [SMILES: COCOC[C@H]1C[C@H]2COc3c(Sc4cnc(N5CCC6(CC5)CO[C@@H](C)[C@H]6N)c(CO)n4)ccnc3N2C1]
in combination with trametinib.

85. The method of claim 83 or 84, wherein the cancer is colorectal cancer.

86. The method of claim 83 or 84, wherein the cancer is lung cancer.

87. The method of claim 83 or 84, wherein the cancer is melanoma.

88. The method of claim 83 or 84, wherein the cancer is pancreatic ductal adenocarcinoma (PDAC).

89. The method of any one of claims 60 to 88, wherein a dosing of the MEK inhibitor is less than a dosing required for a monotherapy with the MEK inhibitor.

90. The method of any one of claims 60 to 89, wherein a dosing of the compound of Formula I is less than a dosing required for a monotherapy with the compound of Formula I.

91. A method of inhibiting ERK1/2 phosphorylation comprising contacting a cell population with Formula I or its pharmaceutically acceptable salt: 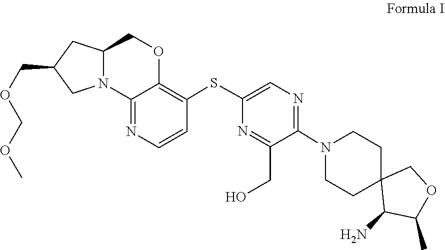 [SMILES: COCOC[C@H]1C[C@H]2COc3c(Sc4cnc(N5CCC6(CC5)CO[C@@H](C)[C@H]6N)c(CO)n4)ccnc3N2C1]
in combination with binimetinib or trametinib.

92. The method of claim 91, wherein a concentration of the compound of Formula I is in a range from 1 nM to 1,000 nM.

93. The method of claim 91 or 92, wherein a concentration of MEK inhibitors is in a range from 10 nM to 500 nM.

94. A kit comprising a compound of Formula I or a pharmaceutically acceptable salt thereof and an MEK inhibitor.

95. The kit of claim 94, wherein the compound of Formula I and the MEK inhibitor are in separate packages.

96. The kit of claim 94 or 95, wherein the kit further comprises instructions to administer the contents of the kit to a subject for the treatment of cancer.

97. The kit of any of claims 94 to 96, wherein the MEK inhibitor is one or more of trametinib or binimetinib.

98. A method of treating a subject having cancer comprising administering to the subject a therapeutically effective amount of a compound of Formula I or its pharmaceutically acceptable salt: 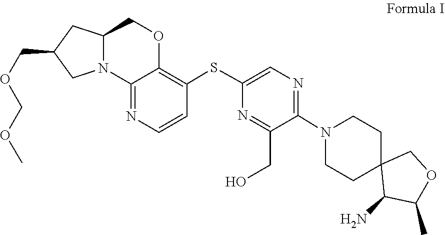 [SMILES: COCOC[C@H]1C[C@H]2COc3c(Sc4cnc(N5CCC6(CC5)CO[C@@H](C)[C@H]6N)c(CO)n4)ccnc3N2C1]
in combination with a MET inhibitor.

99. The method of claim 98, wherein the MET inhibitor is also an ALK inhibitor, a ROS1 inhibitor, or both.

100. The method of claim 98 or 99, wherein the cancer is non-small lung cancer.

101. The method of claim 98 or 99, wherein the cancer is stomach cancer.

102. The method of claim 98 or 99, wherein the cancer is gastric adenocarcinoma.

103. The method of claim 98 or 99, wherein the cancer is pancreatic ductal adenocarcinoma (PDAC).

104. The method of any one of claims 98 to 103, wherein the MET inhibitor is selected from the group consisting of crizotinib, tepotinib, savolitinib, cabozantinib, and tivantinib.

105. The method of any one of claims 98 to 103, wherein the MET inhibitor is crizotinib.

106. The method of any one of claims 98 to 103, wherein the MET inhibitor is tepotinib.

107. The method of any one of claims 98 to 103, wherein the MET inhibitor is savolitinib.

108. The method of any one of claims 98 to 103, wherein the MET inhibitor is cabozantinib.

109. The method of any one of claims 98 to 103, wherein the MET inhibitor is tivantinib.

110. The method of any one of claims 98 to 109, wherein the method comprises administering a third MAPK pathway inhibitor.

111. The method of any one of claims 98 to 110, wherein the administration is oral.

112. The method of any one of claims 98 to 111, wherein the dosing of the compound of Formula I is in a range from 10 mg to 500 mg daily.

113. The method of any one of claims 98 to 112, wherein the dosing of the inhibitor is in a range from 20 mg to 400 mg daily.

114. A method of treating stomach cancer in a subject comprising orally administering to the subject a therapeutically effective amount of a compound of Formula I or its pharmaceutically acceptable salt: 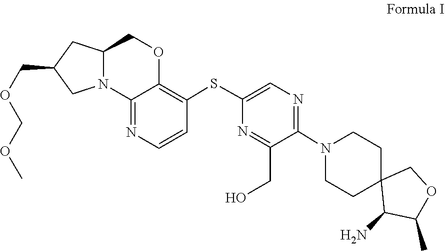 [SMILES: COCOC[C@H]1C[C@H]2COc3c(Sc4cnc(N5CCC6(CC5)CO[C@@H](C)[C@H]6N)c(CO)n4)ccnc3N2C1]
in combination with crizotinib.

115. The method of claim 114, wherein the compound of Formula I is administered once or twice daily.

116. The method of claim 114 or 115, wherein crizotinib is administered once or twice daily.

117. The method of any one of claims 114 to 116, wherein the subject is a human.

118. A kit comprising a compound of Formula I or a pharmaceutically acceptable salt thereof and a MET inhibitor.

119. The kit of claim 118, wherein the compound of Formula I and the MET inhibitor are in separate packages.

120. The kit of claim 118 or 119, wherein the kit further comprises instructions to administer the contents of the kit to a subject for the treatment of cancer.

121. The kit of any of claims 118 to 120, wherein the MET inhibitor is one or more of crizotinib, tepotinib, savolitinib, cabozantinib, and tivantinib.

122. The method of any one of claims 1 to 121, wherein the compound of Formula I, or a pharmaceutically acceptable salt thereof, is formulated as a pharmaceutical composition.

123. The method of any one of claims 1 to 122, wherein the compound of Formula I, or a pharmaceutically acceptable salt thereof, is formulated as an oral composition.

124. The method of any one of claims 1 to 123, wherein the compound of Formula I, or a pharmaceutically acceptable salt thereof, is administered once or twice a day.

125. The method of any one of claims 1 to 124, wherein the compound of Formula I, or a pharmaceutically acceptable salt thereof, is administered over a continuous 28-day cycle.

126. The method of any one of claims 1 to 125, wherein the compound of Formula I, or a pharmaceutically acceptable salt thereof, is administered once a day in the amount of about 10 mg to about 140 mg.

127. The method of any one of claims 1 to 126, wherein the compound of Formula I, or a pharmaceutically acceptable salt thereof, is administered once a day for a 3-week cycle, comprising 2 weeks of administration of the compound followed by 1 week of no administration of the compound.

128. The method of any one of claims 1 to 126, wherein the compound of Formula I, or a pharmaceutically acceptable salt thereof, is administered once a day for a 4-week cycle, comprising 3 weeks of administration of the compound followed by 1 week of no administration of the compound.

129. The method of any one of claims 1 to 128, wherein the compound of Formula I, or a pharmaceutically acceptable salt thereof, is administered over a period of 6 weeks.

130. The method of any one of claims 1 to 128, wherein the compound of Formula I, or a pharmaceutically acceptable salt thereof, is administered over a period of 8 weeks.

131. The method of any one of claims 1 to 130, wherein the compound of Formula I, or a pharmaceutically acceptable salt thereof, is administered 3 times a week.

132. The method of claim 131, wherein the compound of Formula I, or a pharmaceutically acceptable salt thereof, is administered on day 1, day 3, and day 5 of the week.

133. The method of any one of claims 1 to 132, wherein the compound of Formula I, or a pharmaceutically acceptable salt thereof, is administered 4 times a week.

134. The method of claim 133, wherein the compound of Formula I, or a pharmaceutically acceptable salt thereof, is administered for a 3-week cycle, comprising 2 weeks of administration of the compound followed by 1 week of no administration of the compound.

135. The method of claim 133, wherein the compound of Formula I, or a pharmaceutically acceptable salt thereof, is administered for a 4-week cycle, comprising 3 weeks of administration of the compound followed by 1 week of no administration of the compound.

136. The method of any one of claims 1 to 125, wherein the compound of Formula I, or a pharmaceutically acceptable salt thereof, is administered twice a day, two days per week.

137. The method of any one of claims 1 to 126, wherein the compound of Formula I, or a pharmaceutically acceptable salt thereof, is administered over a period of 8 weeks.

138. The method of claim 136 or 137, wherein the compound of Formula I, or a pharmaceutically acceptable salt thereof, is administered on day 1 and day 2 of each week.

139. The method of any one of claims 1 to 138, wherein the cancer is selected from lung cancer, stomach cancer, liver cancer, colon cancer, kidney cancer, breast cancer, pancreatic cancer, pancreatic ductal adenocarcinoma (PDAC), juvenile myelomonocytic leukemia, neurolastoma, melanoma, and acute myeloid leukemia.
